(19) United States
(12) Patent Application Publication
(10) Pub. No.: US 2024/0239806 A1
(43) Pub. Date: Jul. 18, 2024

(54) COMPOUNDS, COMPOSITIONS AND METHODS OF TREATING DISORDERS

(71) Applicant: Chengdu Anticancer Bioscience, Ltd., Chengdu (CN)

(72) Inventors: Dun Yang, Chengdu (CN); Gang Lv, Chengdu (CN); Jing Zhang, Chengdu (CN); Shenqiu Zhang, St. Andrews (GB); Thaddeus Allen, Daly City, CA (US); Qiong Shi, Chengdu (CN); Hongmei Li, Chengdu (CN); Chenglu Yang, Chengdu (CN); Yan Long, Chengdu (CN)

(21) Appl. No.: 18/288,741

(22) PCT Filed: Apr. 29, 2022
(86) PCT No.: PCT/CN2022/090291

§ 371 Date: Oct. 27, 2023

Related U.S. Application Data
(63) Continuation of application No. PCT/CN2021/127309, filed on Oct. 29, 2021.
Application No. PCT/CN2021/127309 is a continuation of application No. PCT/CN2021/091425, filed on Apr. 30, 2021.

(51) Int. Cl. C07D 495/04; A61K 31/4365; A61K 31/4709; A61K 31/506; A61K 31/517; A61K 31/519; A61P 35/00; C07D 215/233; C07D 215/48; C07D 401/12; C07D 401/14; C07D 403/12; C07D 405/04; C07D 405/12; C07D 405/14; C07D 413/12; C07D 487/04

(52) U.S. Cl. CPC C07D 495/04; A61K 31/4365; A61K 31/4709; A61K 31/506; A61K 31/517; A61K 31/519; A61P 35/00; C07D 215/233; C07D 215/48; C07D 401/12; C07D 401/14; C07D 403/12; C07D 405/04; C07D 405/12; C07D 405/14; C07D 413/12; C07D 487/04

(57) ABSTRACT

The present disclose includes, among other things, compounds that treat or lessen the severity of a disorder, pharmaceutical compositions and methods of making and using the same.

### BACKGROUND

Cancer is a term used for diseases in which abnormal cells divide without control and may invade other tissues. Cancer cells may also spread to other parts of the body through the blood and lymph systems.

There are more than 100 different types of cancer, with most cancers named for the organ or type of cell in which they start. For example, cancer that begins in the colon may be referred to as colon cancer; cancer that begins in basal cells of the skin may be referred to as basal cell carcinoma. Common types of cancer include breast cancer and lung cancer.

Cancer types can also be grouped into broader categories. The main categories of cancer include: carcinoma—cancer that begins in the skin or in tissues that line or cover internal organs; sarcoma—cancer that begins in bone, cartilage, fat, muscle, blood vessels, or other connective or supportive tissue; leukemia-cancer that starts in blood-forming tissue such as the bone marrow and causes large numbers of abnormal blood cells to be produced and enter the blood; lymphoma and myeloma—cancers that begin in the cells of the immune system; central nervous system cancers—cancers that begin in the tissues of the brain and spinal cord.

Several techniques for treating cancer are known in the art. Such techniques include chemotherapy, radiation therapy, surgery, and transplantation. Each of these techniques, however, have undesirable side effects and varying success rates. Therefore, a need exists to develop new methods for treating cancer and/or diseases associated with cellular proliferation.

### SUMMARY

The present disclosure provides for compounds of Formula (I)

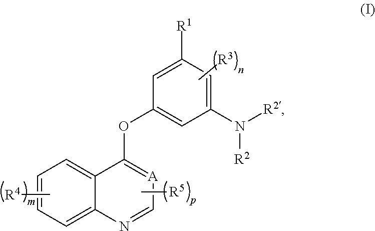

or a pharmaceutically acceptable salt or N-oxide thereof. Additionally, the present disclosure includes, among other things, pharmaceutical compositions, methods of using a compound of Formula (I).

The present disclosure provides for compounds of Formula (II)

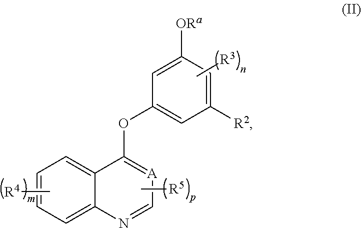

or a pharmaceutically acceptable salt or N-oxide thereof. Additionally, the present disclosure includes, among other things, pharmaceutical compositions, methods of using a compound of Formula (II).

The present disclosure provides for compounds of Formula (III)

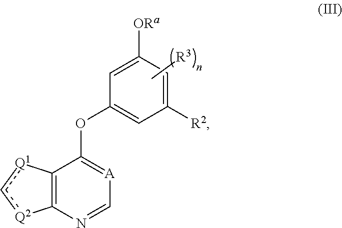

or a pharmaceutically acceptable or N-oxide salt thereof. Additionally, the present disclosure includes, among other things, pharmaceutical compositions, methods of using a compound of Formula (III).

### BRIEF DESCRIPTION OF THE DRAWINGS

FIG. 1 depicts minimal effective concentrations by which 43 compounds elicit polyploidy in the RPEMYCH2B-GFP cell line.

FIG. 2 shows that Compounds #7, #36 and #39 Suppress the Long-Term Proliferative Potential of Cancer Cells in Colony Formation Assays.

FIG. 3A shows that Compounds #7, #8 and #15 Suppress the Anchorage-independent Growth of Human Cancer Cells in 3D Culture.

FIG. 3B shows that Compounds #36, and #39 Suppress the Anchorage-independent Growth of Human Cancer Cells in 3D Culture.

FIG. 4A depicts a graph that shows compounds #21, #26, #40 and #43 suppress the growth of human lung cancer cell line NCI-H23 in immunocompromised mice.

FIG. 4B depicts a graph that shows compounds #21, #26, #40 and #43 suppress the growth of human breast cancer cell line MDA-MB-231 in immunocompromised mice.

### DETAILED DESCRIPTION

The present disclosure includes, among other things, a compound of Formula (I)

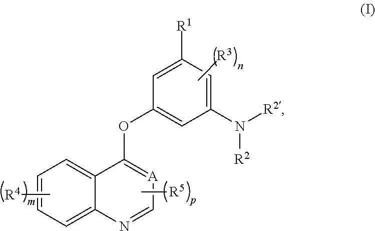

or a pharmaceutically acceptable salt or N-oxide thereof
wherein
A is —C(H)═ or —N═
R¹ is selected from the group consisting of halogen, optionally substituted C₁-C₆ alkyl, optionally substituted C₁-C₆ alkoxy, optionally substituted C₁-C₆ haloalkoxy, —C(O)R^{a}, and —C(O)OR^{a};
R² is selected from the group consisting of hydrogen, optionally substituted C₁-C₆ aliphatic, optionally substituted C₁-C₆ haloaliphatic, —C(O)R^{a}, and —C(O)OR^{a},
R^{2′} is selected from the group consisting of optionally substituted C₁-C₆ aliphatic, optionally substituted C₁-C₆ haloaliphatic, —C(O)R^{a}, and —C(O)OR^{a};
   optionally, R² and R^{2′} are taken together with the nitrogen on which they are attached to form optionally substituted 3-7-membered heterocyclyl or optionally substituted 5-9-membered heteroaryl;
each R³ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, and —C(O)N(R^{a})₂;
each R⁴ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, —C(O)N(R^{a})₂, optionally substituted phenyl, optionally substituted 3-7-membered heterocyclyl and optionally substituted 5-9-membered heteroaryl;
each R⁵ is independently selected from the group consisting of deuterium and halogen;
each R^{a} is independently is selected from the group consisting of hydrogen, optionally substituted C₁-C₆ aliphatic, optionally substituted C₁-C₆ haloaliphatic, optionally substituted 3-7-membered heterocyclyl, optionally substituted 5-9-membered heteroaryl, —C(O)R^{b}, and —C(O)OR^{b};
   optionally, two instances of R^{a} are taken together with the nitrogen on which they are attached to form optionally substituted 3-7-membered heterocyclyl or optionally substituted 5-9-membered heteroaryl;
each R^{b} is independently optionally substituted C₁-C₆ aliphatic;
n is 0, 1, 2, or 3;
m is 0, 1, 2, 3, or 4; and
p is 0, 1, 2, or 3.

Additionally, the present disclosure includes, among other things, a compound of Formula (I)

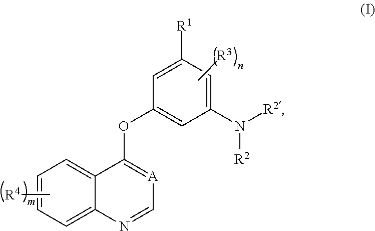

or a pharmaceutically acceptable salt thereof
wherein
A is —C(H)═ or —N═
R¹ is selected from the group consisting of optionally substituted C₁-C₆ alkoxy, optionally substituted C₁-C₆ haloalkoxy, —C(O)R^{a}, and —C(O)OR^{a};
R² is selected from the group consisting of hydrogen, optionally substituted C₁-C₆ aliphatic, optionally substituted C₁-C₆ haloaliphatic, —C(O)R^{a}, and —C(O)OR^{a};
R^{2′} is selected from the group consisting of optionally substituted C₁-C₆ aliphatic, optionally substituted C₁-C₆ haloaliphatic, —C(O)R^{a}, and —C(O)OR^{a};
   optionally, R² and R^{2′} are taken together with the nitrogen on which they are attached to form optionally substituted 3-7-membered heterocyclyl or optionally substituted 5-9-membered heteroaryl;
each R³ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, and —C(O)N(R^{a})₂;
each R⁴ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, —C(O)N(R^{a})₂, optionally substituted phenyl, optionally substituted 3-7-membered heterocyclyl and optionally substituted 5-9-membered heteroaryl
each R^{a} is independently is selected from the group consisting of hydrogen, optionally substituted C₁-C₆ aliphatic, optionally substituted C₁-C₆ haloaliphatic, —C(O)R^{b}, and —C(O)OR^{b};
   optionally, two instances of R^{a} are taken together with the nitrogen on which they are attached to form optionally substituted 3-7-membered heterocyclyl or optionally substituted 5-9-membered heteroaryl;
each R^{b} is independently is selected from the group consisting of optionally substituted C₁-C₆ aliphatic and optionally substituted C₁-C₆ haloaliphatic;
n is 0, 1, 2, or 3;
m is 0, 1, 2, 3, or 4.

In some embodiments, present disclosure includes a compound of formula (I-a):

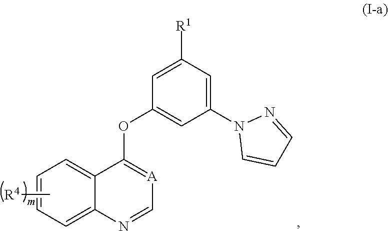

or a pharmaceutically acceptable salt or N-oxide thereof wherein R¹, R⁴, and m are defined above and described in classes and subclasses herein.

In some embodiments, present disclosure includes a compound of formula (I-b)

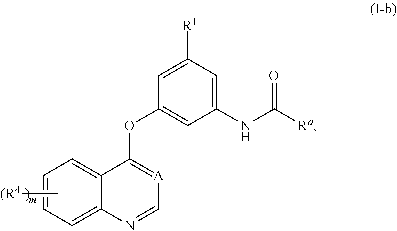

or a pharmaceutically acceptable salt or N-oxide thereof, wherein R¹, R⁴, R^{a}, and m are defined above and described in classes and subclasses herein.

In some embodiments, present disclosure includes a compound of Formula (II)

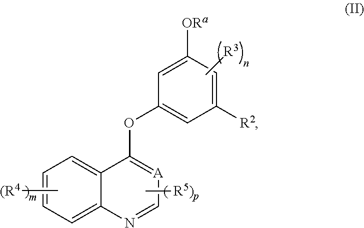

or a pharmaceutically acceptable salt or N-oxide thereof,
wherein
A is —C(H)═ or —N═
R² is selected from the group consisting of —NH₂, —NO₂, —OR^{a}, —O(CH₂)₁₋₃R^{a}, —C(O)OR^{a}, —C(O)N(R^{a})₂, optionally substituted C₁-C₆ aliphatic, and optionally substituted 5-9-membered heteroaryl;
each R³ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, and —C(O)N(R^{a})₂;
each R⁴ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, —C(O)N(R^{a})₂, optionally substituted phenyl, optionally substituted 3-7-membered heterocyclyl and optionally substituted 5-9-membered heteroaryl;
each R⁵ is independently selected from the group consisting of deuterium and halogen;
each R^{a} is independently is selected from the group consisting of hydrogen, optionally substituted C₁-C₆ aliphatic, optionally substituted C₁-C₆ haloaliphatic, optionally substituted 3-7-membered heterocyclyl, optionally substituted 5-9-membered heteroaryl, —C(O)R^{b}, and —C(O)OR^{b};
optionally, two instances of R^{a} are taken together with the nitrogen on which they are attached to form optionally substituted 3-7-membered heterocyclyl or optionally substituted 5-9-membered heteroaryl;
each R^{b} is independently is selected from the group consisting of optionally substituted C₁-C₆ aliphatic and optionally substituted C₁-C₆ haloaliphatic;
n is 0, 1, 2, or 3;
m is 0, 1, 2, 3, or 4; and
p is 0, 1, 2, or 3.

In some embodiments, present disclosure includes a compound of Formula (II):

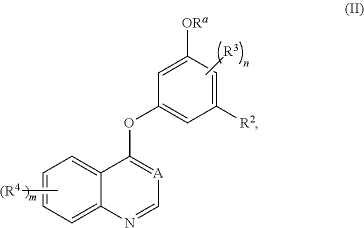

or a pharmaceutically acceptable salt thereof,
wherein
A is —C(H)═ or —N═
R² is selected from the group consisting of —C(O)OR^{a}, —C(O)N(R^{a})₂, optionally substituted C₁-C₆ aliphatic, and optionally substituted 5-9-membered heteroaryl;
each R³ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, and —C(O)N(R^{a})₂;
each R⁴ is independently selected from the group consisting of halogen, —CN, —OR^{a}, —N(R^{a})₂—NO₂, optionally substituted C₁-C₆ aliphatic, optionally substituted C₁-C₆ haloaliphatic, —C(O)R^{a}, —C(O)OR^{a}, —C(O)N(R^{a})₂, optionally substituted phenyl, optionally substituted 3-7-membered heterocyclyl and optionally substituted 5-9-membered heteroaryl
each R^{a} is independently is selected from the group consisting of hydrogen, optionally substituted C₁-C₆ aliphatic, optionally substituted C₁-C₆ haloaliphatic, —C(O)R^{b}, and —C(O)OR^{b};
   optionally, two instances of R^{a} are taken together with the nitrogen on which they are attached to form optionally substituted 3-7-membered heterocyclyl or optionally substituted 5-9-membered heteroaryl;
each R^{b} is independently is selected from the group consisting of optionally substituted C₁-C₆ aliphatic and optionally substituted C₁-C₆ haloaliphatic;
n is 0, 1, 2, or 3;
m is 0, 1, 2, 3, or 4.

In some embodiments, present disclosure includes a compound of formula (II-a):

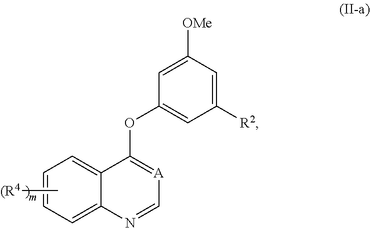

or a pharmaceutically acceptable salt or N-oxide thereof wherein R², R⁴, and m are defined above and described in classes and subclasses herein.

In some embodiments, the present disclosure includes a compound of Formula (II)

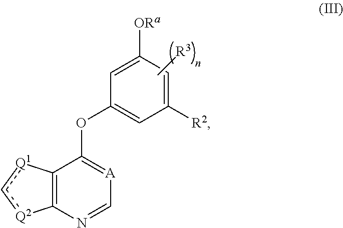

or a pharmaceutically acceptable salt or N-oxide thereof,
wherein
A is —C(H)═ or —N═;
one of Q¹ and Q² is —N(R^{a})— or —S— and the other is —C(H)═;
R² is selected from the group consisting of —C(O)OR^{a}, —C(O)N(R^{a})₂, optionally substituted C₁-C₆ haloaliphatic, and optionally substituted 5-9-membered heteroaryl;
each R³ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, and —C(O)N(R^{a})₂;
each R^{a} is independently is selected from the group consisting of hydrogen, optionally substituted C₁-C₆ aliphatic, optionally substituted C₁-C₆ haloaliphatic, —C(O)R^{b}, and —C(O)OR^{b};
   optionally, two instances of R^{a} are taken together with the nitrogen on which they are attached to form optionally substituted 3-7-membered heterocyclyl or optionally substituted 5-9-membered heteroaryl;
each R^{b} is independently is selected from the group consisting of optionally substituted C₁-C₆ aliphatic and optionally substituted C₁-C₆ haloaliphatic;
n is 0, 1, 2, or 3;
m is 0, 1, 2, 3, or 4.

In some embodiments, present disclosure includes a compound of formula (III-a1) or (III-a2)

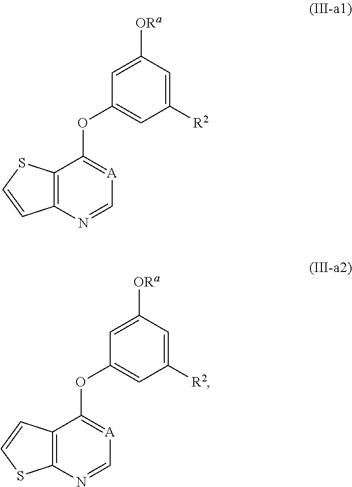

or a pharmaceutically acceptable salt or N-oxide thereof, wherein R^{a} and R² are defined above and described in classes and subclasses herein.

In some embodiments, present disclosure includes a compound of formula (III-b1) or (III-b2)

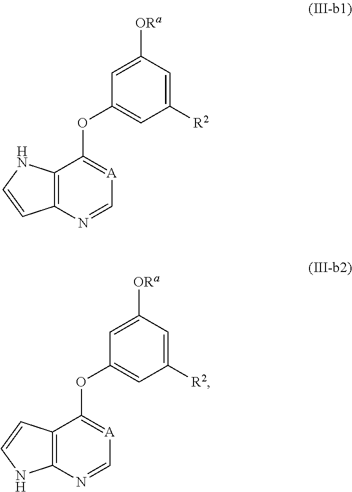

or a pharmaceutically acceptable salt thereof, wherein A, R^{a}, and R² are defined above and described in classes and subclasses herein.

### R¹

In some embodiments, R¹ is selected from the group consisting of halogen optionally substituted C₁-C₆ alkyl, optionally substituted C₁-C₆ alkoxy, optionally substituted C₁-C₆ haloalkoxy, —C(O)R^{a}, and —C(O)OR^{a}. In some embodiments, R¹ is selected from the group consisting of optionally substituted C₁-C₆ alkoxy, optionally substituted C₁-C₆ haloalkoxy, —C(O)R^{a}, and —C(O)OR^{a}. In some embodiments, R¹ is optionally substituted C₁-C₆ alkoxy. In some embodiments, R¹ is —OMe. In some embodiments, R¹ is —C(O)OR^{a}. In some embodiments, R¹ is —C(O)OMe.

R² and R^{2′}

In some embodiments, R² is selected from the group consisting of hydrogen, optionally substituted C₁-C₆ aliphatic, optionally substituted C₁-C₆ haloaliphatic, —C(O)R^{a}, and —C(O)OR^{a}. In some embodiments, R² is selected from the group consisting of —C(O)OR^{a}, —C(O)N(R^{a})₂, optionally substituted C₁-C₆ aliphatic, and optionally substituted 5-9-membered heteroaryl. In some embodiments, R² is selected from the group consisting of —C(O)OR^{a}, —C(O)N(R^{a})₂, optionally substituted C₁-C₆ haloaliphatic, and optionally substituted 5-9-membered heteroaryl;

In some embodiments, R^{2′} is selected from the group consisting of optionally substituted C₁-C₆ aliphatic, optionally substituted C₁-C₆ haloaliphatic, —C(O)R^{a}, and —C(O)OR^{a}.

In some embodiments, R² and R^{2′} are taken together with the nitrogen on which they are attached to form optionally substituted 3-7-membered heterocyclyl or optionally substituted 5-9-membered heteroaryl.

In some embodiments, R² and R^{2′} are taken together with the nitrogen on which they are attached to form o optionally substituted 5-9-membered heteroaryl.

In some embodiments, R² is —C(O)OR^{a}. In some embodiments, R² is —C(O)OR^{a}, and R^{a} of R² is C₁-C₆ aliphatic. In some embodiments, R² is —C(O)OR^{a}, and R^{a} of R² is C₁-C₃ alkyl. In some embodiments, R² is —C(O)OMe.

In some embodiments, R² is —C(O)NHR^{a}, In some embodiments, R² is —C(O)NHR^{a}, and R^{a} of R² is C₁-C₆ aliphatic. In some embodiments, R² is —C(O)NHR^{a}, and R^{a} of R² is C₁-C₃ alkyl.

In some embodiments, R² is —C(O)NHMe.

In some embodiments, R² is optionally substituted 5-membered heteroaryl. In some embodiments, R² is optionally substituted 5-membered heteroaryl comprising 1-3 heteroatoms selected from the group consisting of O, N, and S. In some embodiments, R² is optionally substituted 5-membered heteroaryl comprising 1-3 nitrogen atoms. In some embodiments, R² is optionally substituted oxazolyl or optionally substituted pyrazolyl.

In some embodiments, R² is optionally substituted C₁-C₆ aliphatic. In some embodiments, R² is C₁-C₆ substituted with 1-7 instances of fluoro. In some embodiments, R² is —CF₃.

### R³

In some embodiments, each each R³ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, and —C(O)N(R^{a})₂.

### R⁴

In some embodiments, each R⁴ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, —C(O)N(R^{a})₂, optionally substituted phenyl, optionally substituted 3-7-membered heterocyclyl and optionally substituted 5-9-membered heteroaryl.

### R^{a}

In some embodiments, each R^{a} is independently is selected from the group consisting of hydrogen, optionally substituted C₁-C₆ aliphatic, optionally substituted C₁-C₆ haloaliphatic, optionally substituted 3-7-membered heterocyclyl, optionally substituted 5-9-membered heteroaryl, —C(O)R^{b}, and —C(O)OR^{b}; optionally, two instances of R^{a} are taken together with the nitrogen on which they are attached to form optionally substituted 3-7-membered heterocyclyl or optionally substituted 5-9-membered heteroaryl. In some embodiments, each R^{a} is independently is selected from the group consisting of hydrogen, optionally substituted C₁-C₆ aliphatic, optionally substituted C₁-C₆ haloaliphatic, —C(O)R^{b}, and —C(O)OR^{b}; optionally, two instances of R^{a} am taken together with the nitrogen on which they are attached to form optionally substituted 3-7-membered heterocyclyl or optionally substituted 5-9-membered heteroaryl. In some embodiments, R^{a} is optionally substituted C₁-C₆ aliphatic. In some embodiments, R^{a} is optionally substituted C₁-C₃ alkyl. In some embodiments, R^{a} is optionally substituted methyl.

### R^{b}

In some embodiments, each R^{b} is independently optionally substituted C₁-C₆ aliphatic. In some embodiments, each R^{b} is independently optionally substituted C₁-C₃ alkyl. In some embodiments, R^{b} is independently optionally substituted methyl.

m, and n

In some embodiments, n is 0, 1, 2, or 3. In some embodiments, n is 1, 2, or 3. In some embodiments, m is 0, 1, 2, 3, or 4. In some embodiments, m is 1, 2, 3, or 4.

### Compounds

In some embodiments, compounds of the present disclosure includes those from Table 1.

TABLE 1

| NO. | Structure |
|---|---|
| 1 | 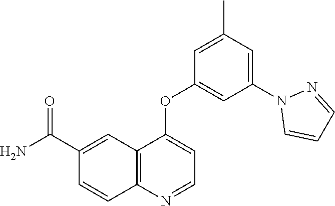 [SMILES: Cc1cc(Oc2ccnc3ccc(C(N)=O)cc23)cc(-n2cccn2)c1] |
| 2 | 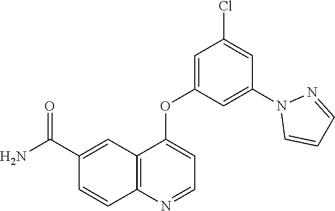 [SMILES: NC(=O)c1ccc2nccc(Oc3cc(Cl)cc(-n4cccn4)c3)c2c1] |
| 3 | 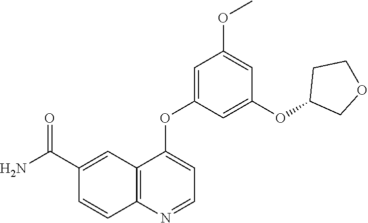 [SMILES: COc1cc(Oc2ccnc3ccc(C(N)=O)cc23)cc(O[C@@H]2CCOC2)c1] |
| 4 | 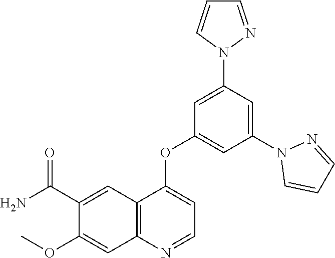 [SMILES: COc1cc2nccc(Oc3cc(-n4cccn4)cc(-n4cccn4)c3)c2cc1C(N)=O] |
| 5 | 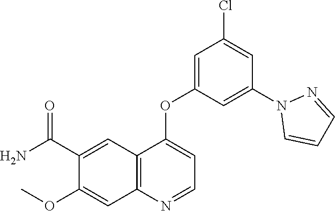 [SMILES: COc1cc2nccc(Oc3cc(Cl)cc(-n4cccn4)c3)c2cc1C(N)=O] |
| 6 | 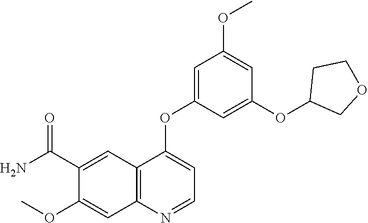 [SMILES: COc1cc(Oc2ccnc3cc(OC)c(C(N)=O)cc23)cc(OC2CCOC2)c1] |
| 7 | 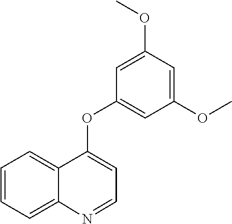 [SMILES: COc1cc(OC)cc(Oc2ccnc3ccccc23)c1] |
| 8 | 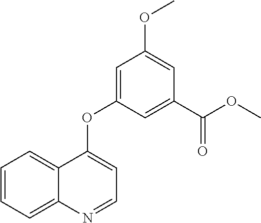 [SMILES: COC(=O)c1cc(OC)cc(Oc2ccnc3ccccc23)c1] |
| 9 | 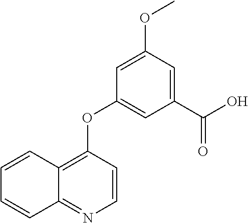 [SMILES: COc1cc(Oc2ccnc3ccccc23)cc(C(=O)O)c1] |
| 10 | 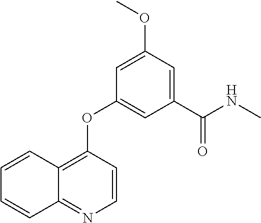 [SMILES: CNC(=O)c1cc(OC)cc(Oc2ccnc3ccccc23)c1] |
| 11 | 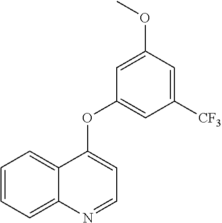 [SMILES: COc1cc(Oc2ccnc3ccccc23)cc(C(F)(F)F)c1] |
| 12 | 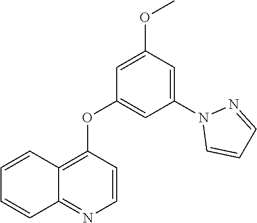 [SMILES: COc1cc(Oc2ccnc3ccccc23)cc(-n2cccn2)c1] |
| 13 | 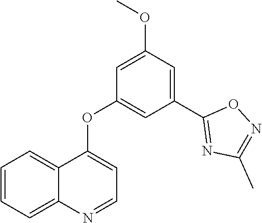 [SMILES: COc1cc(Oc2ccnc3ccccc23)cc(-c2nc(C)no2)c1] |
| 14 | 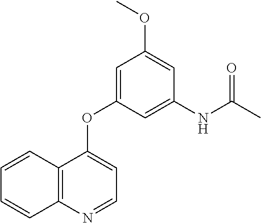 [SMILES: COc1cc(NC(C)=O)cc(Oc2ccnc3ccccc23)c1] |
| 15 | 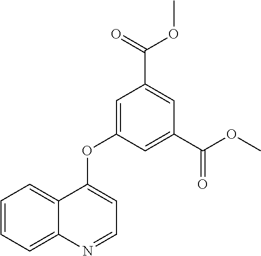 [SMILES: COC(=O)c1cc(Oc2ccnc3ccccc23)cc(C(=O)OC)c1] |
| 17 | 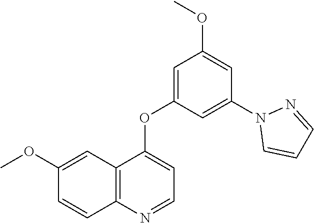 [SMILES: COc1cc(Oc2ccnc3ccc(OC)cc23)cc(-n2cccn2)c1] |
| 18 | 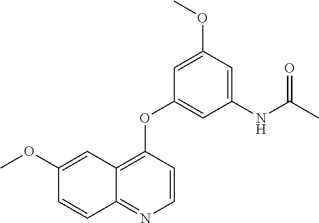 [SMILES: COc1cc(NC(C)=O)cc(Oc2ccnc3ccc(OC)cc23)c1] |
| 19 | 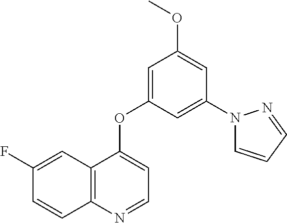 [SMILES: COc1cc(Oc2ccnc3ccc(F)cc23)cc(-n2cccn2)c1] |
| 20 | 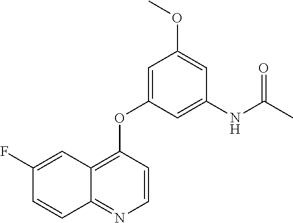 [SMILES: COc1cc(NC(C)=O)cc(Oc2ccnc3ccc(F)cc23)c1] |
| 21 | 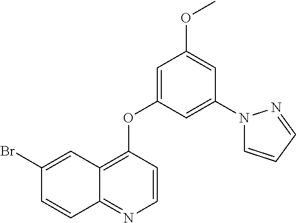 [SMILES: COc1cc(Oc2ccnc3ccc(Br)cc23)cc(-n2cccn2)c1] |
| 22 | 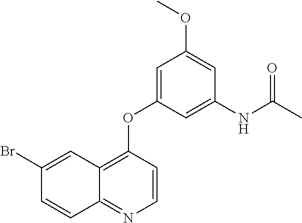 [SMILES: COc1cc(NC(C)=O)cc(Oc2ccnc3ccc(Br)cc23)c1] |
| 26 | 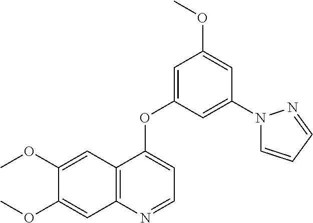 [SMILES: COc1cc(Oc2ccnc3cc(OC)c(OC)cc23)cc(-n2cccn2)c1] |
| 27 | 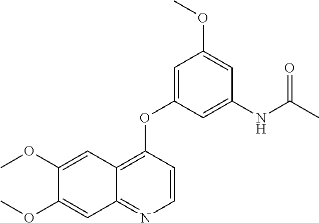 [SMILES: COc1cc(NC(C)=O)cc(Oc2ccnc3cc(OC)c(OC)cc23)c1] |
| 29 | 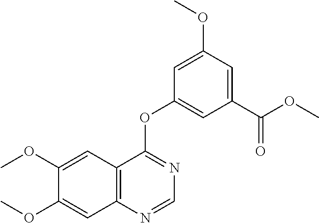 |
| 30 | 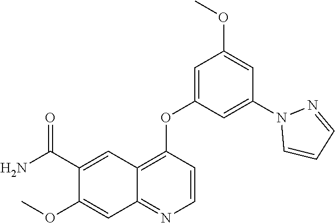 [SMILES: COc1cc(Oc2ccnc3cc(OC)c(C(N)=O)cc23)cc(-n2cccn2)c1] |
| 31 | 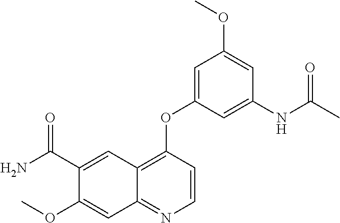 [SMILES: COc1cc(NC(C)=O)cc(Oc2ccnc3cc(OC)c(C(N)=O)cc23)c1] |
| 32 | 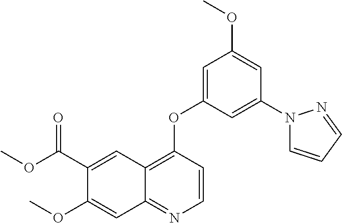 [SMILES: COC(=O)c1cc2c(Oc3cc(OC)cc(-n4cccn4)c3)ccnc2cc1OC] |
| 34 | 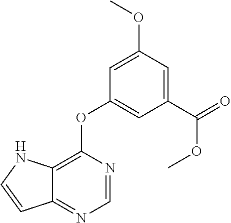 [SMILES: COC(=O)c1cc(OC)cc(Oc2ncnc3cc[nH]c23)c1] |
| 35 | 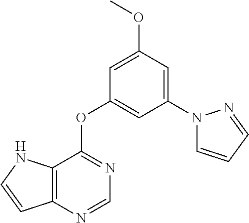 [SMILES: COc1cc(Oc2ncnc3cc[nH]c23)cc(-n2cccn2)c1] |
| 36 | 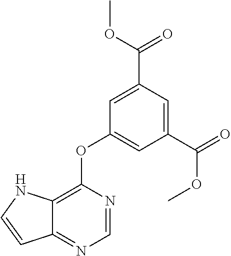 [SMILES: COC(=O)c1cc(Oc2ncnc3cc[nH]c23)cc(C(=O)OC)c1] |
| 37 | 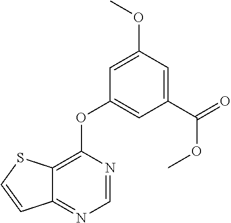 [SMILES: COC(=O)c1cc(OC)cc(Oc2ncnc3ccsc23)c1] |
| 38 | 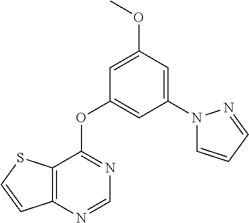 [SMILES: COc1cc(Oc2ncnc3ccsc23)cc(-n2cccn2)c1] |
| 39 | 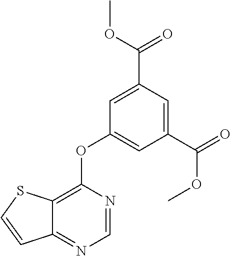 [SMILES: COC(=O)c1cc(Oc2ncnc3ccsc23)cc(C(=O)OC)c1] |
| 40 | 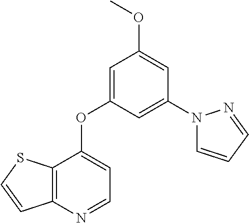 [SMILES: COc1cc(Oc2ccnc3ccsc23)cc(-n2cccn2)c1] |
| 41 | 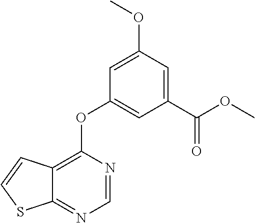 [SMILES: COC(=O)c1cc(OC)cc(Oc2ncnc3sccc23)c1] |
| 42 | 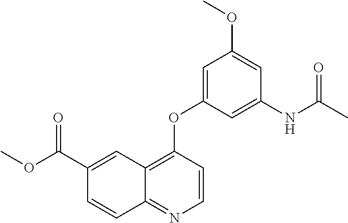 [SMILES: COC(=O)c1ccc2nccc(Oc3cc(NC(C)=O)cc(OC)c3)c2c1] |
| 43 | 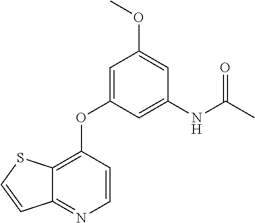 [SMILES: COc1cc(NC(C)=O)cc(Oc2ccnc3ccsc23)c1] |
| 44 | 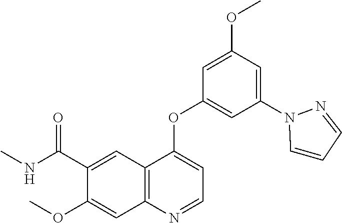 [SMILES: CNC(=O)c1cc2c(Oc3cc(OC)cc(-n4cccn4)c3)ccnc2cc1OC] |
| 45 | 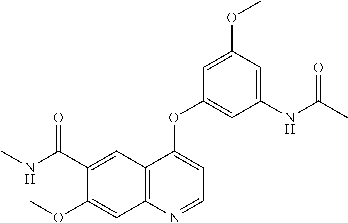 [SMILES: CNC(=O)c1cc2c(Oc3cc(NC(C)=O)cc(OC)c3)ccnc2cc1OC] |
| 46 | 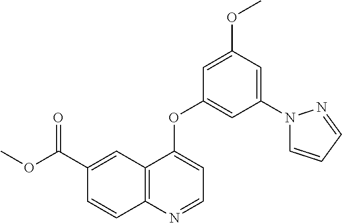 [SMILES: COC(=O)c1ccc2nccc(Oc3cc(OC)cc(-n4cccn4)c3)c2c1] |
| 47 | 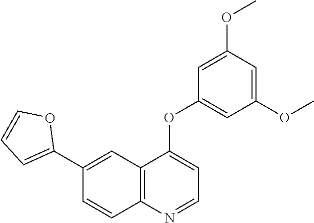 [SMILES: COc1cc(OC)cc(Oc2ccnc3ccc(-c4ccco4)cc23)c1] |
| 48 | 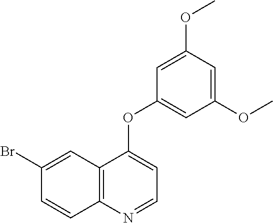 [SMILES: COc1cc(OC)cc(Oc2ccnc3ccc(Br)cc23)c1] |
| 49 | 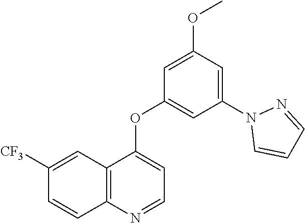 [SMILES: COc1cc(Oc2ccnc3ccc(C(F)(F)F)cc23)cc(-n2cccn2)c1] |
| 50 | 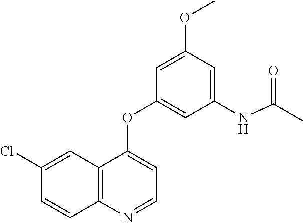 [SMILES: COc1cc(NC(C)=O)cc(Oc2ccnc3ccc(Cl)cc23)c1] |
| 51 | 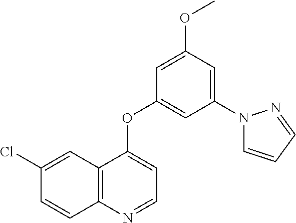 [SMILES: COc1cc(Oc2ccnc3ccc(Cl)cc23)cc(-n2cccn2)c1] |
| 52 | 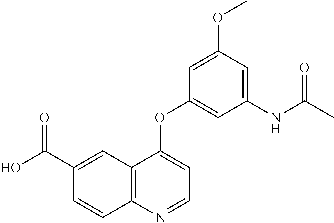 [SMILES: COc1cc(NC(C)=O)cc(Oc2ccnc3ccc(C(=O)O)cc23)c1] |
| 53 | 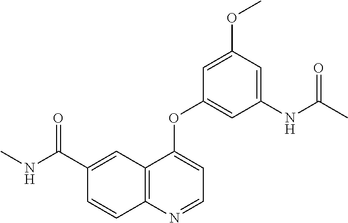 [SMILES: CNC(=O)c1ccc2nccc(Oc3cc(NC(C)=O)cc(OC)c3)c2c1] |
| 54 | 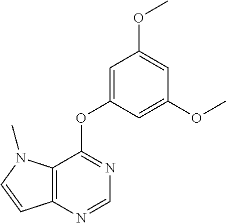 [SMILES: COc1cc(OC)cc(Oc2ncnc3ccn(C)c23)c1] |
| 55 | 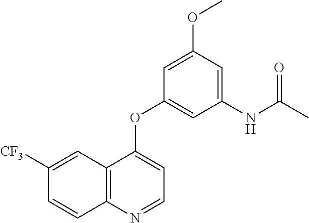 [SMILES: COc1cc(NC(C)=O)cc(Oc2ccnc3ccc(C(F)(F)F)cc23)c1] |
| 56 | 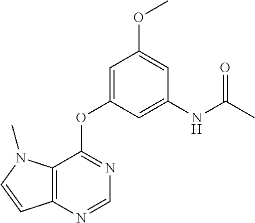 [SMILES: COc1cc(NC(C)=O)cc(Oc2ncnc3ccn(C)c23)c1] |
| 57 | 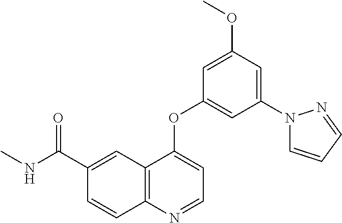 [SMILES: CNC(=O)c1ccc2nccc(Oc3cc(OC)cc(-n4cccn4)c3)c2c1] |
| 58 | 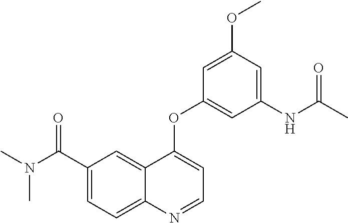 [SMILES: COc1cc(NC(C)=O)cc(Oc2ccnc3ccc(C(=O)N(C)C)cc23)c1] |
| 59 | 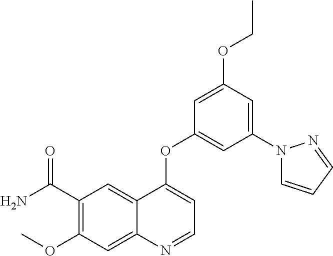 [SMILES: CCOc1cc(Oc2ccnc3cc(OC)c(C(N)=O)cc23)cc(-n2cccn2)c1] |
| 60 | 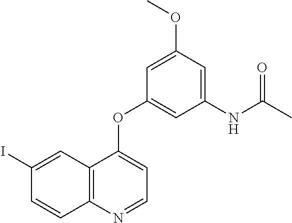 [SMILES: COc1cc(NC(C)=O)cc(Oc2ccnc3ccc(I)cc23)c1] |
| 61 | 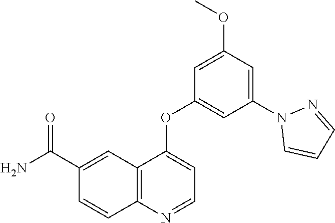 [SMILES: COc1cc(Oc2ccnc3ccc(C(N)=O)cc23)cc(-n2cccn2)c1] |
| 62 | 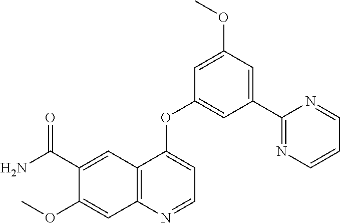 [SMILES: COc1cc(Oc2ccnc3cc(OC)c(C(N)=O)cc23)cc(-c2ncccn2)c1] |
| 63 | 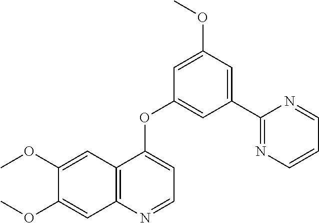 [SMILES: COc1cc(Oc2ccnc3cc(OC)c(OC)cc23)cc(-c2ncccn2)c1] |
| 64 | 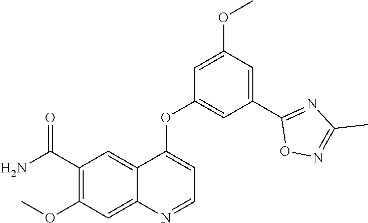 [SMILES: COc1cc(Oc2ccnc3cc(OC)c(C(N)=O)cc23)cc(-c2nc(C)no2)c1] |
| 65 | 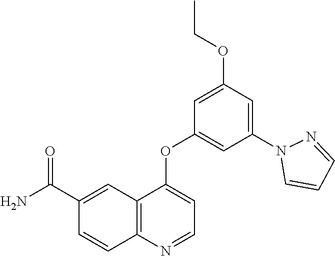 [SMILES: CCOc1cc(Oc2ccnc3ccc(C(N)=O)cc23)cc(-n2cccn2)c1] |
| 66 | 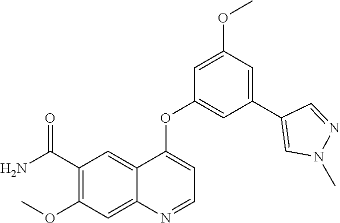 [SMILES: COc1cc(Oc2ccnc3cc(OC)c(C(N)=O)cc23)cc(-c2cnn(C)c2)c1] |
| 67 | 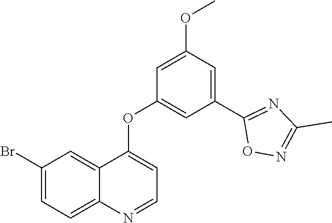 [SMILES: COc1cc(Oc2ccnc3ccc(Br)cc23)cc(-c2nc(C)no2)c1] |
| 68 | 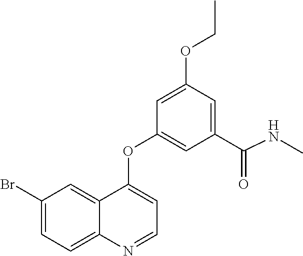 [SMILES: CCOc1cc(Oc2ccnc3ccc(Br)cc23)cc(C(=O)NC)c1] |
| 69 | 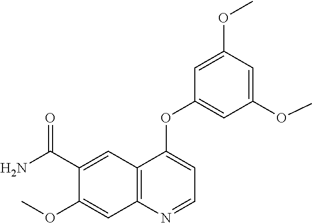 [SMILES: COc1cc(OC)cc(Oc2ccnc3cc(OC)c(C(N)=O)cc23)c1] |
| 70 | 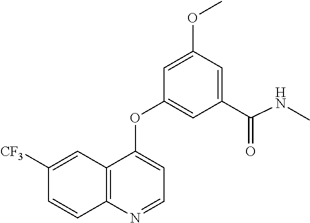 [SMILES: CNC(=O)c1cc(OC)cc(Oc2ccnc3ccc(C(F)(F)F)cc23)c1] |
| 71 | 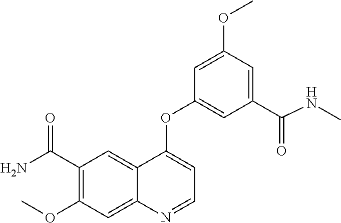 |
| 72 | 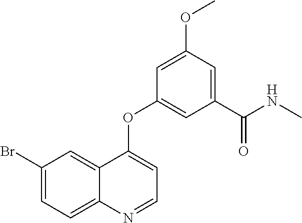 [SMILES: CNC(=O)c1cc(OC)cc(Oc2ccnc3ccc(Br)cc23)c1] |
| 73 | 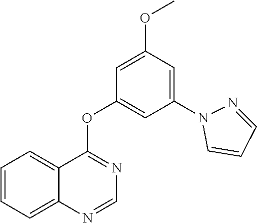 [SMILES: COc1cc(Oc2ncnc3ccccc23)cc(-n2cccn2)c1] |
| 74 | 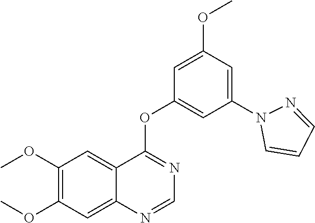 [SMILES: COc1cc(Oc2ncnc3cc(OC)c(OC)cc23)cc(-n2cccn2)c1] |
| 75 | 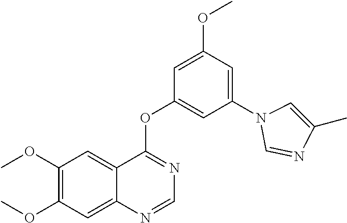 |
| 76 | 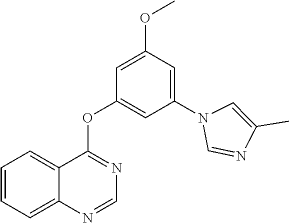 [SMILES: COc1cc(Oc2ncnc3ccccc23)cc(-n2cnc(C)c2)c1] |
| 77 | 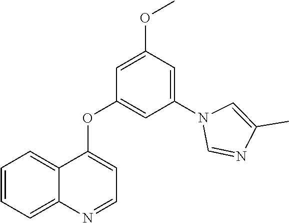 [SMILES: COc1cc(Oc2ccnc3ccccc23)cc(-n2cnc(C)c2)c1] |
| 78 | 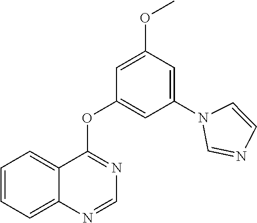 [SMILES: COc1cc(Oc2ncnc3ccccc23)cc(-n2ccnc2)c1] |
| 79 | 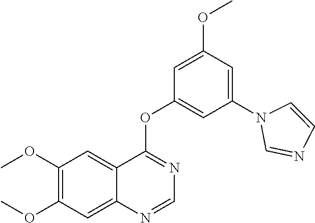 [SMILES: COc1cc(Oc2ncnc3cc(OC)c(OC)cc23)cc(-n2ccnc2)c1] |
| 80 | 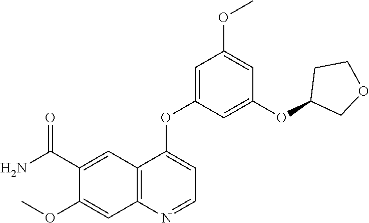 [SMILES: COc1cc(Oc2ccnc3cc(OC)c(C(N)=O)cc23)cc(O[C@H]2CCOC2)c1] |
| 81 | 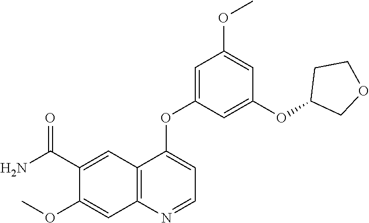 [SMILES: COc1cc(Oc2ccnc3cc(OC)c(C(N)=O)cc23)cc(O[C@@H]2CCOC2)c1] |
| 82 | 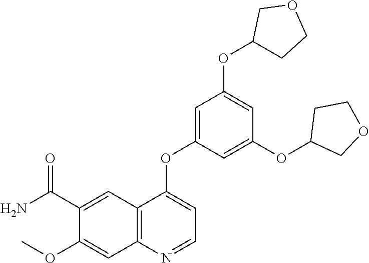 [SMILES: COc1cc2nccc(Oc3cc(OC4CCOC4)cc(OC4CCOC4)c3)c2cc1C(N)=O] |
| 83 | 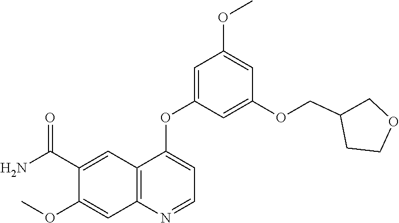 [SMILES: COc1cc(OCC2CCOC2)cc(Oc2ccnc3cc(OC)c(C(N)=O)cc23)c1] |
| 84 | 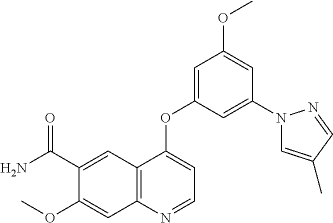 [SMILES: COc1cc(Oc2ccnc3cc(OC)c(C(N)=O)cc23)cc(-n2cc(C)cn2)c1] |
| 85 | 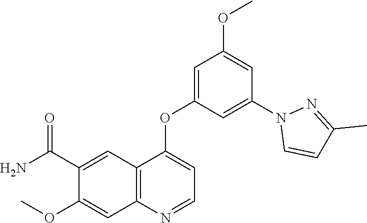 [SMILES: COc1cc(Oc2ccnc3cc(OC)c(C(N)=O)cc23)cc(-n2ccc(C)n2)c1] |
| 86 | 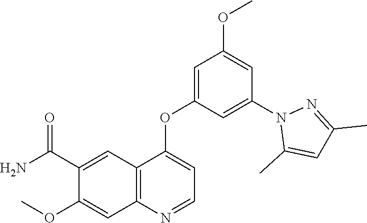 [SMILES: COc1cc(Oc2ccnc3cc(OC)c(C(N)=O)cc23)cc(-n2nc(C)cc2C)c1] |
| 87 | 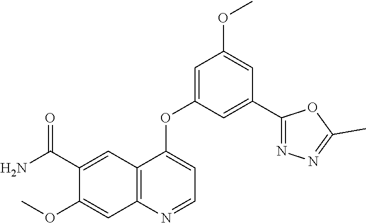 [SMILES: COc1cc(Oc2ccnc3cc(OC)c(C(N)=O)cc23)cc(-c2nnc(C)o2)c1] |
| 88 | 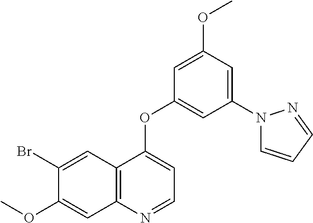 [SMILES: COc1cc(Oc2ccnc3cc(OC)c(Br)cc23)cc(-n2cccn2)c1] |
| 89 | 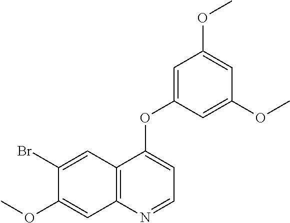 |
| 90 | 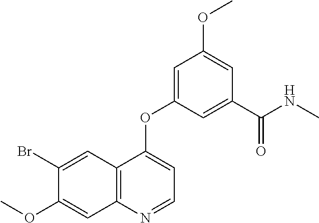 |
| 91 | 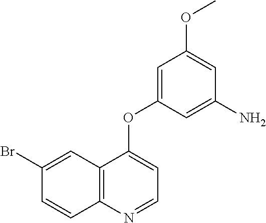 [SMILES: COc1cc(N)cc(Oc2ccnc3ccc(Br)cc23)c1] |
| 92 | 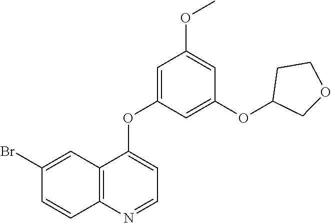 [SMILES: COc1cc(Oc2ccnc3ccc(Br)cc23)cc(OC2CCOC2)c1] |
| 93 | 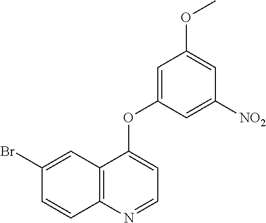 [SMILES: COc1cc(Oc2ccnc3ccc(Br)cc23)cc([N+](=O)[O-])c1] |
| 94 | 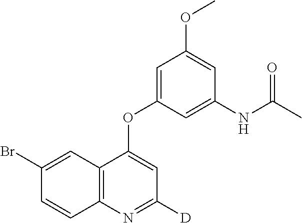 [SMILES: [2H]c1cc(Oc2cc(NC(C)=O)cc(OC)c2)c2cc(Br)ccc2n1] |
| 95 | 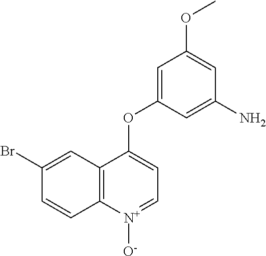 [SMILES: COc1cc(N)cc(Oc2cc[n+]([O-])c3ccc(Br)cc23)c1] |
| 96 | 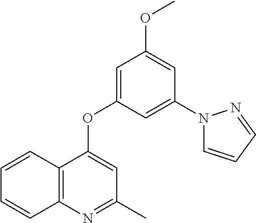 [SMILES: COc1cc(Oc2cc(C)nc3ccccc23)cc(-n2cccn2)c1] |
| 97 | 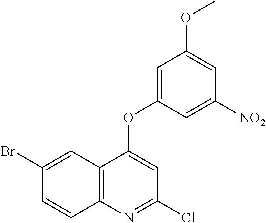 [SMILES: COc1cc(Oc2cc(Cl)nc3ccc(Br)cc23)cc([N+](=O)[O-])c1] |
| 98 | 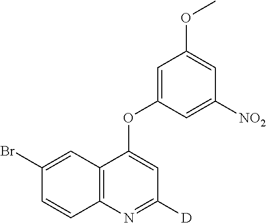 [SMILES: [2H]c1cc(Oc2cc(OC)cc([N+](=O)[O-])c2)c2cc(Br)ccc2n1] |

or a pharmaceutically acceptable salt thereof.

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “carbocyclyl” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl” refers to a straight or branched alkyl group. Exemplary alkyl groups are methyl, ethyl, propyl, isopropyl, butyl, isobutyl, and tert-butyl.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —SF₅, —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)_{0- 4}N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)S(O)₂OR^{∘}; —(CH₂)₀₋₄OS(O)₂R^{∘}; —S(O)₂NR^{∘}₂; —(CH₂)₀₋₄S(O)R^{∘}; —S(O)(NR^{∘})R^{∘}, —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen. —SF₅, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —N₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —SF₅, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —S(O)(NR^{†})R^{†}, —C(S)NR^{†}₂, —C(NH)NR^{†}₂, or —N(R^{†})S(O)₂R^{†}; wherein each R^{†} s independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —SF₅, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As used herein, “N-oxide” refers to the oxide of the nitrogen atom of a nitrogen-containing heteroaryl or heterocycle. N-oxide can be formed in the presence of an oxidizing agent such as m-chloroperbenzoic acid or hydrogen peroxide. N-oxide refers to an amine oxide, also known as amine-N-oxide, and is a chemical compound that contains N—O bond.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof mixtures of enantiomers or diasteromers may be designated “(f)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with TUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. Substituents around a carbocyclic or heterocyclic ring may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diasteriomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

Additionally, the present disclosure contemplates tautomers of the compounds as drawn herein.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. I

As used herein, the terms “treatment,” “treat,” and “treating” refer to partially or completely alleviating, inhibiting, delaying onset of, ameliorating and/or relieving a disorder or condition, or one or more symptoms of the disorder or condition, as described herein. In some embodiments, treatment may be administered after one or more symptoms have developed. In some embodiments, the term “treating” includes halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder, activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably treat a disease or disorder in a biological sample or in a patient. In certain embodiments, the amount of compound in compositions of this disclosure is such that is effective to measurably treat a disease or disorder in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (I) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (I) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (I) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (I) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (I) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

In some embodiments, the present disclosure provides a method for treating or lessening the severity of a disease or condition associated with cell proliferation in a patient comprising the step of administering to said patient a composition according to the present disclosure.

The term “disease or condition associated with cell proliferation”, as used herein means any disease or other deleterious condition in which cell proliferation is known to play a role. Accordingly, another embodiment of the present disclosure relates to treating or lessening the severity of one or more diseases in which cell proliferation is known to play a role. In some embodiments, a disease or condition associated with cell proliferation is cancer.

In some embodiments, administration of a compound of the present disclosure results in arrest of mitosis or change in DNA content.

In some embodiments, administration of a compound of the present disclosure results in arrest of mitosis. In some embodiments, mitotic arrest is defined as a 10-100% reduction in mitosis. In some embodiments, mitotic arrest is defined as a 20-100% reduction in mitosis. In some embodiments, mitotic arrest is defined as a 30-100% reduction in mitosis. In some embodiments, mitotic arrest is defined as a 40-100% reduction in mitosis. In some embodiments, mitotic arrest is defined as a 50-100% reduction in mitosis. In some embodiments, mitotic arrest is defined as a 60-100% reduction in mitosis. In some embodiments, mitotic arrest is defined as a 70-100% reduction in mitosis. In some embodiments, mitotic arrest is defined as a 80-100% reduction in mitosis. In some embodiments, mitotic arrest is defined as a 90-100% reduction in mitosis. In some embodiments, mitotic arrest is defined as a 100% reduction in mitosis.

In some embodiments, administration of a compound of the present disclosure results in change in DNA content. In some embodiments, change of DNA content is induction of polyploidy.

In some embodiments, compounds and compositions, according to a method of the present disclosure, may be administered using any amount and any route of administration effective for treating or lessening the severity of cancer. The exact amount required will vary from subject to subject, depending on the species, age, and general condition of the subject, severity of the infection, particular agent, its mode of administration, and the like. Compounds of the present disclosure are preferably formulated in dosage unit form for ease of administration and uniformity of dosage.

In some embodiments, cancer is selected from the group consisting of lung cancer and breast cancer. In some embodiments, cancer is lung cancer. In some embodiments, lung cancer is non-small cell lung cancer. In some embodiments, non-small cell lung cancer is lung adenocarcinoma. In some embodiments, cancer is breast cancer. In some embodiments, breast cancer is mammary cancer. In some embodiments, breast cancer is breast adenocarcinoma.

In some embodiments, pharmaceutically acceptable compositions of comprising compounds of the present disclosure can be administered to humans and other animals orally, rectally, parenterally, intracisternally, intravaginally, intraperitoneally, topically (as by powders, ointments, or drops), buccally, as an oral or nasal spray, or the like, depending on the severity of infection being treated. In certain embodiments, compounds of the present disclose may be administered orally or parenterally at dosage levels of about 0.01 mg/kg to about 50 mg/kg and preferably from about 1 mg/kg to about 25 mg/kg, of subject body weight per day, one or more times a day, to obtain desired therapeutic effect.

In some embodiments, one or more additional therapeutic agents, may also be administered in combination with compounds of the present disclosure. In some embodiments, a compound of the present disclosure and one or more additional therapeutic agents may be administered as part of a multiple dosage regime. In some embodiments, a compound of the present disclosure and one or more additional therapeutic agents may be administered may be administered simultaneously, sequentially or within a period of time. In some embodiments, a compound of the present disclosure and one or more additional therapeutic agents may be administered within five hours of one another. In some embodiments, a compound of the present disclosure and one or more additional therapeutic agents may be administered within 24 hours of one another. In some embodiments, a compound of the present disclosure and one or more additional therapeutic agents may be administered within one week of one another.

In some embodiments, a compound of the present disclosure and one or more additional therapeutic agents may be formulated into a single dosage form.

### Exemplification

### General Methods

Unless stated otherwise, all the chemicals required for synthesis were purchased from commercially available suppliers and used without further purification. ¹H NMR spectra was determined with a Bruker Avance III-400 at 400 MHz. LC-MS analysis was performed on a platform equipped with Agilent LC-MS 1260-6110 or Agilent LC-MS 1260-6120, using a Waters X Bridge C18: 50 mm×4.6 mm×3.5 um column. Flash column chromatography was conducted with silica gel (200-300 mesh, Qingdao Haiyang Chemical Co. Ltd., China). Analytical and preparative TLC analysis were performed on GF254 silica gel plates (Yantai Jiangyou Inc., China). Unless otherwise noted, reagents and all solvents are analytically pure grade and were obtained commercially from vendors such as Chron Chemical or Energy-Chemical.

Abbreviations: TLC: Thin Layer Chromatography, EA: Ethyl Acetate, PE: Petroleum Ether, DMF: N,N-dimethylformamide, THF: Eetrahydrofuran, DCM: Dichloromethane, DIPEA: N,N-diisopropylethylamine,

DMAP: 4-dimethylaminopyridine

### Example 1. 4-(3-methyl-5-(1H-pyrazol-1-yl)phenoxy)quinoline-6-carboxamide (Compound 1)

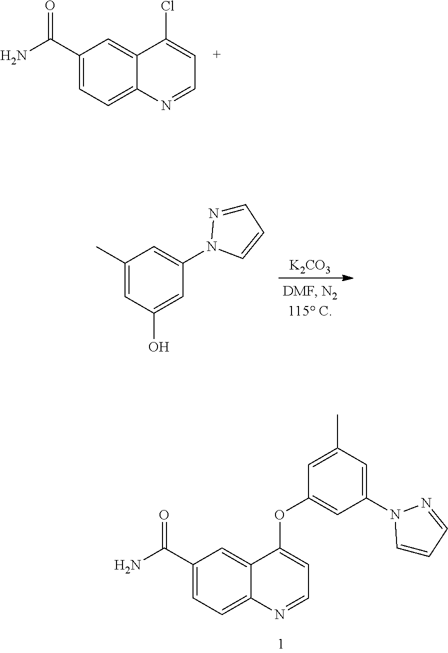

4-4-chloroquinoline-6-carboxamide (103.3 mg, 0.5 mmol, 1.0 eq), 3-methyl-5-(1H-pyrazol-1-yl)phenol (130.5 mg, 0.75 mmol, 1.5 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (156 mg, yield=90.6%, purity=93.2%)

TLC R_{f}=0.4 (DCM/MeOH=20/1)

MS (ESI⁺): m/z=345.10 (M+1)

¹H NMR: (400 MHz, DMSO-d6) δ 8.93 (d, J=1.9 Hz, 1H), 8.79 (d, J=5.2 Hz, 1H), 8.58 (dd, J=2.6, 0.6 Hz, 1H), 8.36 (s, 1H), 8.29 (dd, J=8.8, 2.0 Hz, 1H), 8.10 (d, J=8.9 Hz, 1H), 7.76 (d, J=1.8 Hz, 1H), 7.74 (q, J=1.1 Hz, 1H), 7.66 (t, J=2.3 Hz, 1H), 7.59 (s, 1H), 7.11 (p, J=0.9 Hz, 1H), 6.82 (d, J=5.2 Hz, 1H), 6.56 (dd, J=2.6, 1.7 Hz, 1H), 2.45 (s, 3H).

### Example 2. 4-(3-chloro-5-(1H-pyrazol-1-yl)phenoxy)quinoline-6-carboxamide (Compound 2)

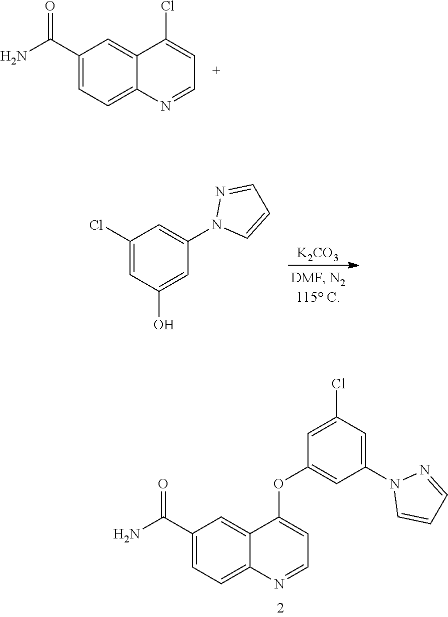

4-chloroquinoline-6-carboxamide (103.3 mg, 0.5 mmol, 1.0 eq), 3-chloro-5-(1H-pyrazol-1-yl)phenol (117 mg, 0.6 mmol, 1.2 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (160 mg, yield=87.7%, purity=97.8%)

TLC R_{f}=0.25 (DCM/MeOH=20/1)

MS (ESI⁺): m/z=365.20 (M+1)

### Example 3. (R)-4-(methoxy-5-((tetrahydrofuran-3-yl)oxy)phenoxy)quinoline-6-carboxamide (Compound 3)

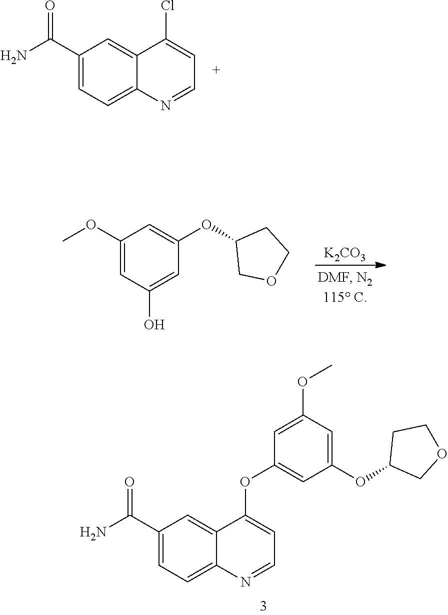

4-chloroquinoline-6-carboxamide (103.3 mg, 0.5 mmol, 1.0 eq), (R)-3-methoxy-5-((tetrahydrofuran-3-yl)oxy)phenol (210 mg, 1.0 mmol, 2.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (138 mg, yield=72.6%, purity=97.6%)

TLC R_{f}=0.2 (PE/EA=1/8)

MS (ESI⁺): m/z=381.40 (M+1)

¹H NMR: (400 MHz, DMSO-d6) δ 8.82 (d, J=1.9 Hz, 1H), 8.71 (d, J=5.2 Hz, 1H), 8.27 (s, 1H), 8.20 (dd, J=8.8, 2.0 Hz, 1H), 8.00 (d, J=8.8 Hz, 1H), 7.50 (s, 1H), 6.71 (d, J=5.2 Hz, 1H), 6.46 (t, J=2.1 Hz, 1H), 6.44 (t, J=2.1 Hz, 11H), 6.41 (t, J=2.2 Hz, 1H), 3.81-3.75 (m, 2H), 3.74 (t, J=1.8 Hz, 1H), 3.70 (s, 3H), 3.73-3.63 (m, 2H), 2.22-2.03 (m, 1H), 1.97-1.85 (m, 1H).

### Example 4. 4-(3,5-di(1H-pyrazol-1-yl)phenoxy)-7-methoxyquinoline-6-carboxamide (Compound 4)

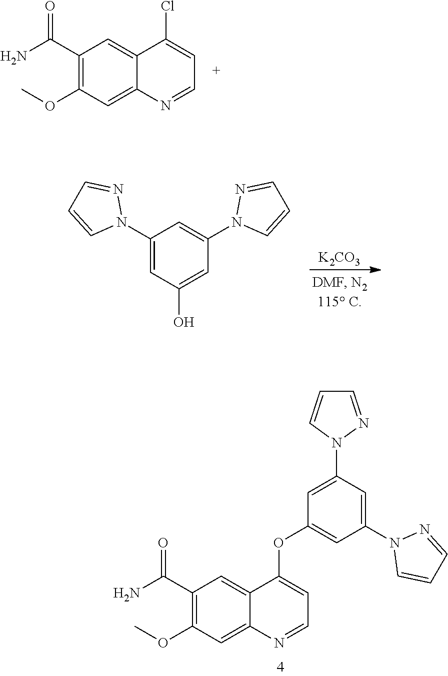

4-chloro-7-methoxyquinoline-6-carboxamide (142 mg, 0.6 mmol, 1.0 eq), 3,5-di(1H-pyrazol-1-yl)phenol (203 mg, 0.9 mmol, 1.5 eq) and K₂CO₃ (331 mg, 2.4 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (77 mg, yield=85.5%, purity=91.7%)

TLC R_{f}=0.4 (DCM/MeOH=20/1)

MS (ESI⁺): m/z=427.10 (M+1)

¹H NMR: (400 MHz, DMSO-d6) δ 8.67 (d, J=1.4 Hz, 2H), 8.66 (s, 1H), 8.63 (d, J=2.6 Hz, 2H), 8.30 (t, J=2.0 Hz, 1H), 7.88-7.78 (m, 1H), 7.75 (d, J=1.7 Hz, 2H), 7.69 (d, J=2.0 Hz, 2H), 7.50 (s, 1H), 6.75 (d, J=5.3 Hz, 1H), 6.54 (dd, J=2.6, 1.7 Hz, 2H), 3.99 (s, 3H).

### Example 5. 4-(3-chloro-5-(1H-pyrazol-1-yl)phenoxy)-7-methoxyquinoline-6-carboxamide (Compound 5)

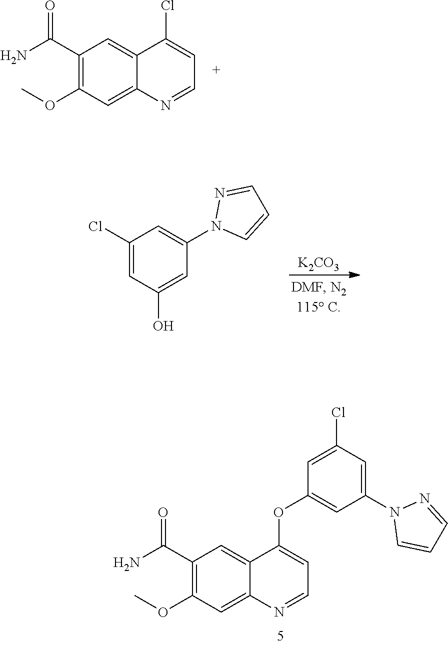

4-chloro-7-methoxyquinoline-6-carboxamide (119 mg, 0.5 mmol, 1.0 eq), 3-chloro-5-(1H-pyrazol-1-yl)phenol (117 mg, 0.6 mmol, 1.2 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (175 mg, yield=88.6%, purity=97.2%)

TLC R_{f}=0.5 (DCM/MeOH=20/1)

MS (ESI⁺): m/z=395.30 (M+1)

### Example 6. 7-methoxy-4-(3-methoxy-5-((tetrahydrofuran-3-yl)oxy)phenoxy)quinoline-6-carboxamide (Compound 6)

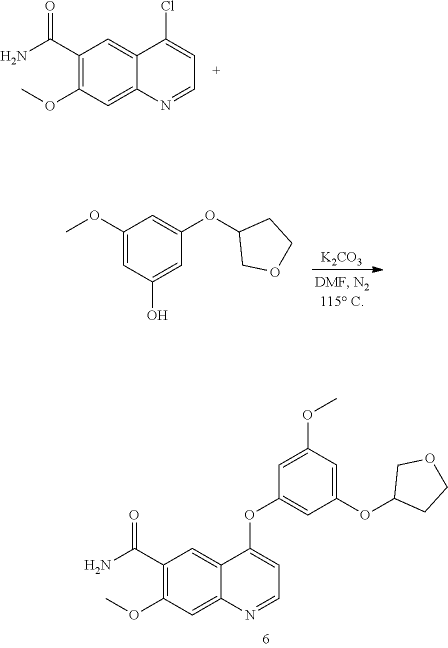

4-chloro-7-methoxyquinoline-6-carboxamide (119 mg, 0.5 mmol, 1.0 eq), 3-methoxy-5-((tetrahydrofuran-3-yl)oxy)phenol (210 mg, 1.0 mmol, 2.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (205 mg, yield=99%, purity=93.9%)

TLC R_{f}=0.45 (DCM/MeOH=20/1)

MS (ESI⁺): m/z=411.50 (M+1)

¹H NMR: (400 MHz, DMSO-d6) δ 8.69 (d, J=5.2 Hz, 1H), 8.67 (s, 1H), 7.86 (s, 1H), 7.75 (s, 1H), 7.52 (s, 1H), 6.63 (d, J=5.2 Hz, 1H), 6.50 (t, J=2.1 Hz, 1H), 6.47 (p, J=2.2 Hz, 2H), 5.06 (ddt, J=6.3, 4.1, 1.8 Hz, 1H), 4.04 (s, 3H), 3.88-3.82 (m, 2H), 3.81 (q, J=1.5 Hz, 1H), 3.76 (s, 3H), 3.73 (dt, J=8.3, 4.2 Hz, 1H), 2.20 (dtd, J=13.4, 8.2, 6.2 Hz, 1H), 2.03-1.92 (m, 1H).

### Example 7. 4-(3,5-dimethoxyphenoxy)quinoline (Compound 7)

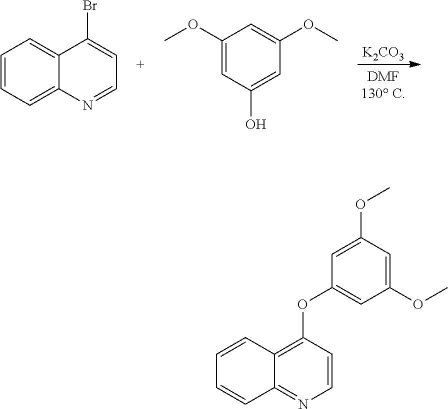

4-bromoquinoline (104 mg, 0.5 mmol, 1.0 eq), 3,5-dimethoxyphenol (92.4 mg, 0.6 mmol, 1.2 eq) and K₂CO₃ (138 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (111 mg, yield=79.3%, purity=99%)

TLC R_{f}=0.3 (PE/EA=2/1)

MS (ESI⁺): m/z=282.50 (M+1)

¹H NMR: (400 MHz, CDCl₃-d) δ 8.72 (d, J=5.2 Hz, 1H), 8.36 (ddd, J=8.4, 1.5, 0.6 Hz, 1H), 8.14 (dt, J=8.5, 0.9 Hz, 1H), 7.79 (ddd, J=8.5, 6.9, 1.5 Hz, 1H), 7.61 (ddd, J=8.2, 6.8, 1.2 Hz, 1H), 6.70 (d, J=5.2 Hz, 1H), 6.42 (t, J=2.2 Hz, 1H), 6.37 (d, J=2.2 Hz, 2H), 3.81 (s, 6H). ¹³C NMR: (101 MHz, DMSO) δ 161.59, 160.54, 155.57, 151.45, 149.21, 130.16, 128.78, 126.29, 121.40, 120.63, 104.72, 99.25, 97.85, 55.51.

### Example 8. Methyl 3-methoxy-5-(quinolin-4-yloxy)benzoate (Compound 8)

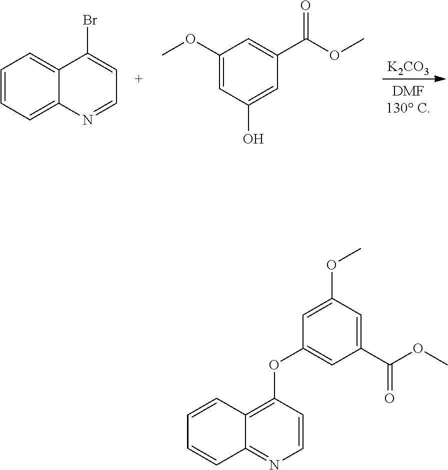

4-bromoquinoline (104 mg, 0.5 mmol, 1.0 eq), methyl 3-hydroxy-5-methoxybenzoate (109 mg, 0.6 mmol, 1.2 eq) and K₂CO₃ (138 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (112 mg, yield=72.5%, purity=98.6%)

TLC R_{f}=0.33 (PE/EA=3/1)

MS (ESI⁺): m/z=310.80 (M+1)

¹H NMR: (400 MHz, DMSO-d6) δ 8.72 (d, J=5.1 Hz, 1H), 8.27 (ddd, J=8.3, 1.5, 0.7 Hz, 1H), 8.05 (dt, J=8.4, 1.0 Hz, 1H), 7.83 (ddd, J=8.5, 6.9, 1.5 Hz, 1H), 7.65 (ddd, J=8.3, 6.9, 1.2 Hz, 1H), 7.40 (dd, J=2.4, 1.3 Hz, 1H), 7.31 (dd, J=2.2, 1.3 Hz, 1H), 7.23 (t, J=2.3 Hz, 1H), 6.73 (d, J=5.1 Hz, 1H), 3.84 (d, J=6.0 Hz, 6H). ¹³C NMR: (101 MHz, DMSO) δ 165.16, 160.96, 160.21, 155.25, 151.43, 149.29, 132.47, 130.29, 128.82, 126.49, 121.37, 120.65, 113.04, 111.64, 105.21, 55.87, 52.44.

### Example 9. 3-methoxy-5-(quinolin-4-yloxy) benzoic add (Compound 9)

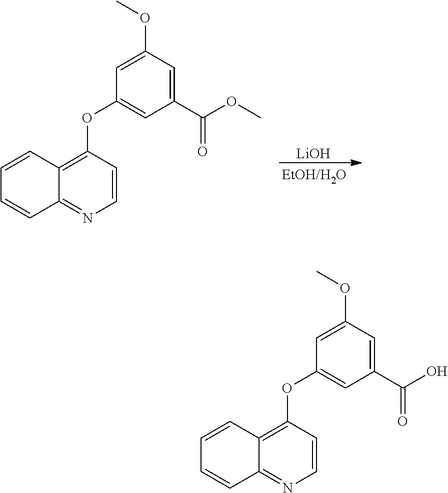

Methyl 3-methoxy-5-(quinolin-4-yloxy) benzoate (50 mg, 0.162 mmol, 1.0 eq) and LiOH (20.4 mg, 0.485 mmol, 3.0 eq) were added to a round-bottom flask with a magnetic bat. Then 0.6 ml EtOH and 0.3 ml H₂O were added as solvent. The reaction mixture was stirred overnight. When methyl 3-methoxy-5-(quinolin-4-yloxy)benzoate was consumed, the pH of reaction mixture was adjusted to 7 and some white solid formed, which was filtered and dried to give the product without further purification. (42 mg, yield=87.8%, purity=99%)

MS (ESI⁺): m/z=296.40 (M+1)

¹H NMR: (400 MHz, DMSO-d6) δ 9.05 (d, J=6.4 Hz, 1H), 8.57 (dd, J=8.5, 1.2 Hz, 11H), 8.51 (d, J=8.6 Hz, 11H), 8.21 (ddd, J=8.5, 7.0, 1.3 Hz, 11H), 8.05-7.89 (m, 1H), 7.50 (p, J=1.3 Hz, 2H), 7.39 (t, J=2.3 Hz, 1H), 7.05 (d, J=6.4 Hz, 1H), 3.86 (s, 3H). ¹³C NMR: (101 MHz, DMSO) δ 167.29, 166.37, 161.54, 153.89, 147.09, 140.19, 135.18, 134.84, 129.65, 123.42, 121.31, 120.71, 114.26, 113.90, 112.17, 105.23, 56.52.

### Example 10. 3-methoxy-N-methyl-5-(quinolin-4-yloxy)benzamide (Compound 10)

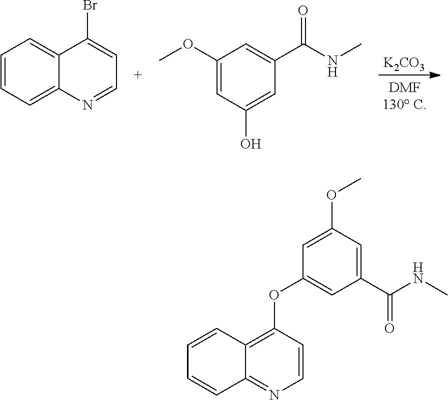

4-bromoquinoline (104 mg, 0.5 mmol, 1.0 eq), 3-hydroxy-5-methoxy-N-methylbenzamide (90.5 mg, 0.5 mmol, 1.0 eq) and K₂CO₃ (138 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (127.9 mg, yield=83%, purity=99%)

TLC R_{f}=0.2 (DCM/MeOH=40/1)

MS (ESI⁺): m/z=309.40 (M+1)

¹H NMR: (400 MHz, DMSO-d6) δ 8.74 (d, J=5.1 Hz, 1H), 8.52 (q, J=4.5 Hz, 1H), 8.30 (dd, J=8.4, 1.4 Hz, 1H), 8.09-8.04 (m, 1H), 7.84 (ddd, J=8.5, 6.9, 1.5 Hz, 1H), 7.67 (ddd, J=8.2, 6.9, 1.2 Hz, 1H), 7.40 (dd, J=2.4, 1.4 Hz, 1H), 7.30 (t, J=1.7 Hz, 1H), 7.10 (t, J=2.3 Hz, 1H), 6.73 (d, J=5.1 Hz, 1H), 3.85 (s, 3H), 2.77 (d, J=4.5 Hz, 3H). ¹³C NMR: (101 MHz, DMSO) δ 165.64, 161.31, 160.91, 155.48, 151.98, 149.75, 137.86, 130.78, 129.32, 126.98, 121.87, 121.17, 111.79, 110.67, 109.93, 105.62, 56.26, 26.75.

### Example 11. 4-(3-methoxy-5-trifluoromethyl)phenoxy)quinoline (Compound 11)

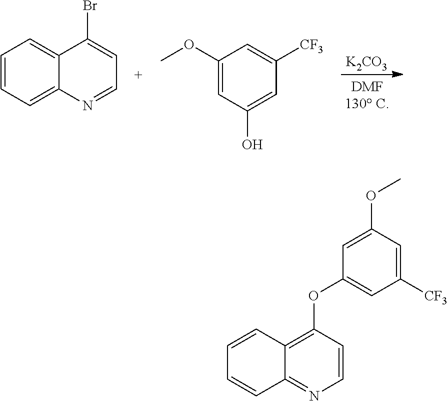

4-bromoquinoline (104 mg, 0.5 mmol, 1.0 eq), 3-methoxy-5-(trifluoromethyl)phenol (96 mg, 0.5 mmol, 1.0 eq) and K₂CO₃ (138 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (92 mg, yield=57.7%, purity=99%)

TLC R_{f}=0.2 (PE/EA=4/1)

MS (ESI⁺): m/z=320.10 (M+1)

¹H NMR: (400 MHz, DMSO-d6) δ 8.75 (d, J=5.1 Hz, 1H), 8.29 (dd, J=8.4, 1.4 Hz, 1H), 8.10-8.03 (m, 1H), 7.84 (ddd, J=8.4, 6.8, 1.5 Hz, 1H), 7.67 (ddd, J=8.2, 6.9, 1.2 Hz, 1H), 7.25 (s, 3H), 6.76 (d, J=5.1 Hz, 1H), 3.87 (s, 3H). ¹³C NMR: (101 MHz, DMSO) δ 161.89, 160.58, 156.11, 151.96, 149.79, 132.44, 132.12, 130.80, 129.32, 127.00, 121.87, 121.10, 111.25, 110.12, 108.94, 105.65, 56.61.

### Example 12. 4-(3-methoxy-5-(1H-pyrazol-1-yl)phenoxy)quinoline (Compound 12)

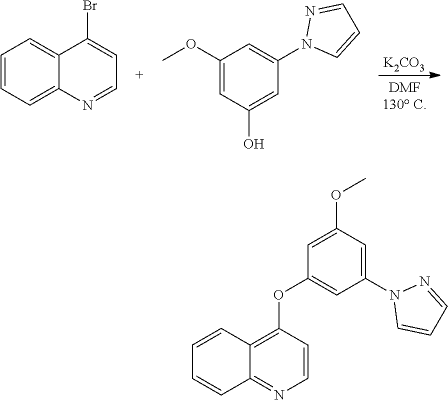

4-bromoquinoline (104 mg, 0.5 mmol, 1.0 eq), 3-methoxy-5-(1H-pyrazol-1-yl)phenol (95 mg, 0.5 mmol, 1.0 eq) and K₂CO₃ (138 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (134 mg, yield=84.4%, purity=99%)

TLC R_{f}=0.2 (PE/EA=2/1)

MS (ESI⁺): m/z=318.40 (M+1)

¹H NMR: (400 MHz, DMSO-d6) δ 8.74 (d, J=5.1 Hz, 1H), 8.62 (d, J=2.6 Hz, 1H), 8.32 (dd, J=8.4, 1.4 Hz, 1H), 8.07 (d, J=8.4 Hz, 1H), 7.84 (ddd, J=8.4, 6.8, 1.5 Hz, 1H), 7.75 (d, J=1.6 Hz, 1H), 7.68 (ddd, J=8.2, 6.9, 1.2 Hz, 1H), 7.45 (t, J=2.1 Hz, 1H), 7.41 (t, J=2.0 Hz, 1H), 6.86 (t, J=2.2 Hz, 1H), 6.83 (d, J=5.1 Hz, 1H), 6.55 (dd, J=2.6, 1.7 Hz, 1H), 3.87 (s, 3H). ¹³C NMR: (101 MHz, DMSO) δ 162.08, 160.88, 156.26, 152.00, 149.76, 142.34, 141.76, 130.77, 129.32, 128.73, 126.96, 121.91, 121.16, 108.67, 105.64, 104.75, 103.40, 101.90, 56.38.

### Example 13. 5-(3-methoxy-5-(quinolin-4-yloxy)phenyl)-3-methyl-oxadiazole (Compound 13)

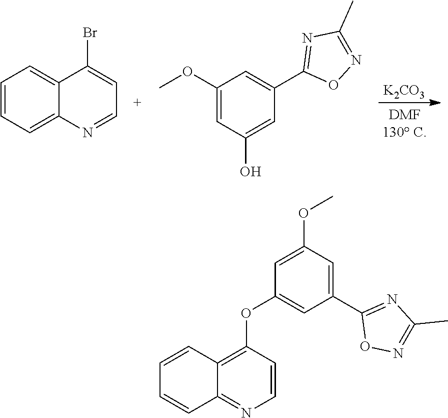

4-bromoquinoline (104 mg, 0.5 mmol, 1.0 eq), 3-methoxy-5-(3-methyl-1,2,4-oxadiazol-5-yl)phenol (103 mg, 0.5 mmol, 1.0 eq) and K₂CO₃ (138 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (158 mg, yield=94.8%, purity=93.5%)

TLC R_{f}=0.45 (PE/MA=1/1)

MS (ESI⁺): m/z=334.40 (M+1)

¹H NMR: (400 MHz, DMSO-d6) δ 8.75 (d, J=5.1 Hz, 1H), 8.29 (dd, J=8.4, 1.4 Hz, 1H), 8.07 (d, J=8.4 Hz, 1H), 7.84 (ddd, J=8.5, 6.9, 1.5 Hz, 1H), 7.67 (ddd, J=8.2, 6.9, 1.2 Hz, 1H), 7.51 (dd, J=2.4, 1.4 Hz, 1H), 7.47-7.42 (m, 1H), 7.26 (t, J=2.3 Hz, 1H), 6.83 (d, J=5.1 Hz, 1H), 3.89 (s, 3H), 2.40 (s, 3H). ¹³C NMR: (101 MHz, DMSO) δ 174.26,168.24,161.95, 160.56, 156.38, 151.92, 149.75, 130.87, 129.28, 127.08, 126.36, 121.90, 121.19, 112.10, 111.84, 110.67, 106.04, 56.53, 11.67.

### Example 14. N-(3-methoxy-5-(quinolin-4-yloxy)phenyl)acetamide (Compound 14)

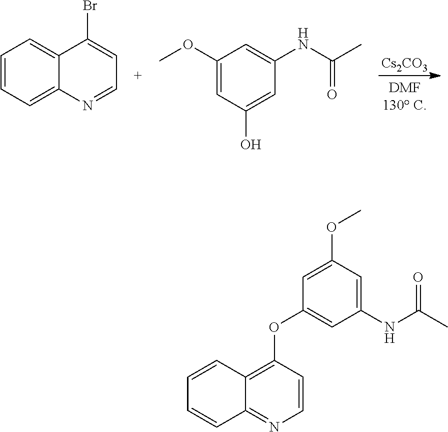

4-bromoquinoline (124.8 mg, 0.6 mmol, 1.0 eq), N-(3-hydroxy-5-methoxyphenyl)acetamide (90.5 mg, 0.5 mmol, 1.0 eq) and Cs₂CO₃ (326 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (141 mg, yield=91.6%, purity=99.52%)

TLC R_{f}=0.1 (PE/EA=1/2)

MS (ESI⁺): m/z=309.40 (M+1)

¹H NMR: (400 MHz, DMSO-d6) δ 10.12 (s, 1H), 8.74 (d, J=5.2 Hz, 1H), 8.28 (d, J=8.3 Hz, 1H), 8.05 (d, J=8.4 Hz, 1H), 7.89-7.79 (m, 1H), 7.66 (t, J=7.6 Hz, 1H), 7.15 (dt, J=10.4, 2.0 Hz, 2H), 6.76 (d, J=5.1 Hz, 1H), 6.61 (t, J=2.3 Hz, 1H), 3.76 (s, 3H), 2.04 (s, 3H). ¹³C NMR: (101 MHz, DMSO) δ 169.13, 161.40, 161.01, 155.63, 151.91, 149.65, 142.13, 130.78, 129.23, 126.95, 121.91, 121.21, 105.62, 103.66, 102.32, 101.58, 55.89, 24.59.

### Example 15. dimethyl 5-(quinolin-4-yloxy)isophthalate (Compound 15)

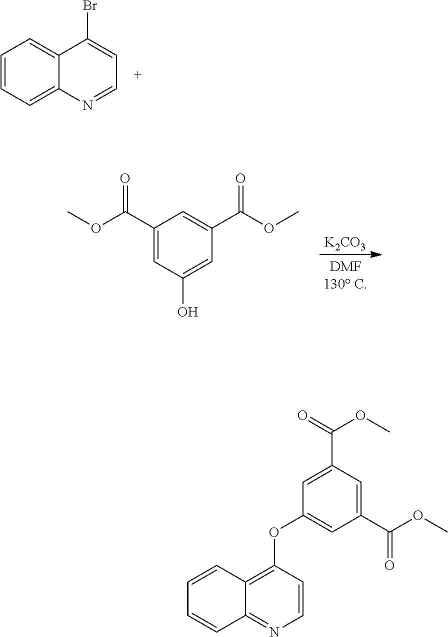

4-bromoquinoline (125 mg, 0.6 mmol, 1.0 eq), dimethyl 5-hydroxyisophthalate (126 mg, 0.6 mmol, 1.0 eq) and K₂CO₃ (331 mg, 2.4 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (49 mg, yield=24.23%, purity=99%)

TLC R_{f}=0.2 (PE/EA=2/1)

MS (ESI⁺): m/z=338.30 (M+1)

¹H NMR: (400 MHz, DMSO-d6) δ 8.75 (d, J=5.1 Hz, 1H), 8.39 (t, J=1.5 Hz, 1H), 8.29 (dd, J=8.4, 1.4 Hz, 1H), 8.08 (d, J=8.4 Hz, 1H), 8.03 (d, J=1.5 Hz, 21H), 7.86 (ddd, J=8.4, 6.9, 1.5 Hz, 111), 7.69 (ddd, J=8.2, 6.9, 1.2 Hz, 1H), 6.81 (d, J=5.1 Hz, 11H), 3.90 (s, 6H). ¹³C NMR: (101 MHz, DMSO) δ 165.05, 160.46, 155.26, 151.95, 149.85, 133.04, 130.97, 129.36, 127.22, 126.82, 125.92, 121.93, 121.18, 106.22, 53.23.

### Example 16. 6-methoxy-4(3-methoxy-5-(1H-pyrazol-1-yl)phenoxy)quinoline (Compound 17)

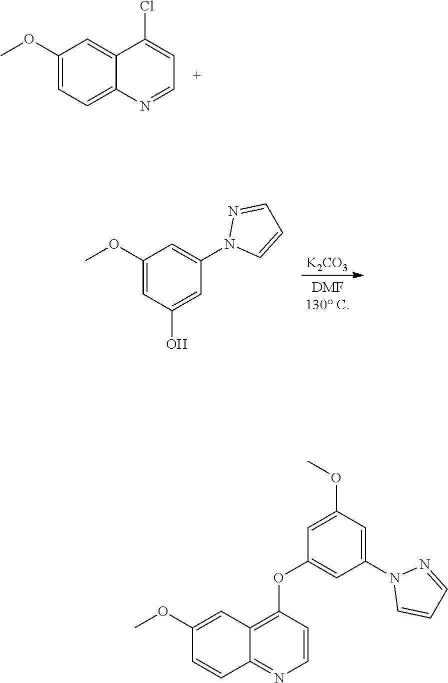

4-chloro-6-methoxyquinoline (87.2 mg, 0.45 mmol, 1.0 eq), 3-methoxy-5-(1H-pyrazol-1-yl)phenol (85.6 mg, 0.45 mmol, 1.0 eq) and K₂CO₃ (124.2 mg, 0.9 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (72 mg, yield=46.83%, purity=99%)

TLC R_{f}=0.45 (PE/EA=1/1)

MS (ESI⁺): m/z=348.60 (M+1)

¹H NMR: (400 MHz, DMSO-d6) δ 8.62 (d, J=2.6 Hz, 1H), 8.58 (d, J=5.1 Hz, 1H), 7.97 (d, J=9.2 Hz, 1H), 7.75 (d, J=1.7 Hz, 1H), 7.57 (d, J=2.8 Hz, 1H), 7.48 (dd, J=9.2, 2.9 Hz, 1H), 7.45 (t, J=2.1 Hz, 1H), 7.40 (t, J=2.0 Hz, 1H), 6.85 (t, J=2.2 Hz, 1H), 6.79 (d, J=5.1 Hz, 1H), 6.55 (dd, J=2.5, 1.7 Hz, 1H), 3.93 (a, 3H), 3.87 (s, 3H). ¹³C NMR: (101 MHz, DMSO) δ 162.06, 159.94, 157.83, 156.35, 149.23, 145.82, 142.32, 141.75, 131.02, 128.72, 123.11, 121.99, 108.67, 105.93, 104.74, 103.43, 101.83, 99.56, 56.37, 56.04.

### Example 17. N-(3-methoxy-5-((6-methoxyquinolin-4-yl)oxy)phenyl)acetamide (Compound 18)

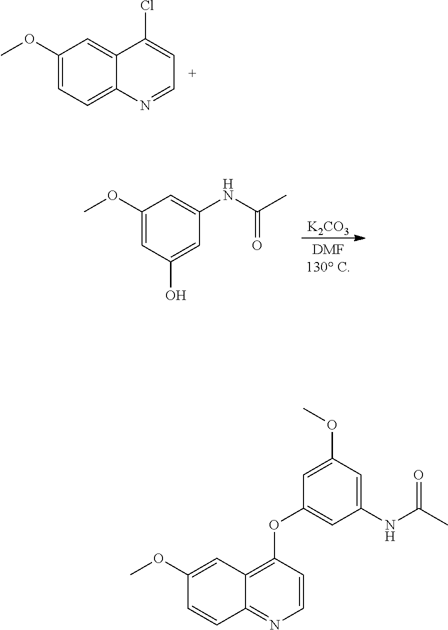

4-chloro-6-methoxyquinoline (97 mg, 0.5 mmol, 1.0 eq), N-(3-hydroxy-5-methoxyphenyl)acetamide (91 mg, 0.5 mmol, 1.0 eq) and K₂CO₃ (138 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (120 mg, yield=70.9%, purity=98.6%)

TLC R_{f}=0.2 (PE/EA=1/4)

MS (ESI): m/z=339.60 (M+1)

¹H NMR: (400 MHz, DMSO-d6) δ 10.10 (s, 1H), 8.57 (d, J=5.1 Hz, 11H), 7.96 (d, J=9.2 Hz, 1H), 7.53 (d, J=2.9 Hz, 1H), 7.46 (dd, J=9.2, 2.9 Hz, 1H), 7.14 (d, J=2.2 Hz, 2H), 6.71 (d, J=5.1 Hz, 1H), 6.60 (t, J=2.2 Hz, 1H), 3.93 (s, 3H), 3.76 (s, 3H), 2.03 (s, 3H). ¹³C NMR: (101 MHz, DMSO) δ 169.11, 161.37, 160.03, 157.80, 155.72, 149.16, 145.77, 142.08, 130.98, 123.07, 122.03, 105.88, 103.73, 102.24, 101.62, 99.52, 56.01, 55.37, 24.58.

### Example 18. 6-fluoro-4-(3-methoxy-5-(1H-pyrazol-1-yl)phenoxy)quinoline (Compound 19)

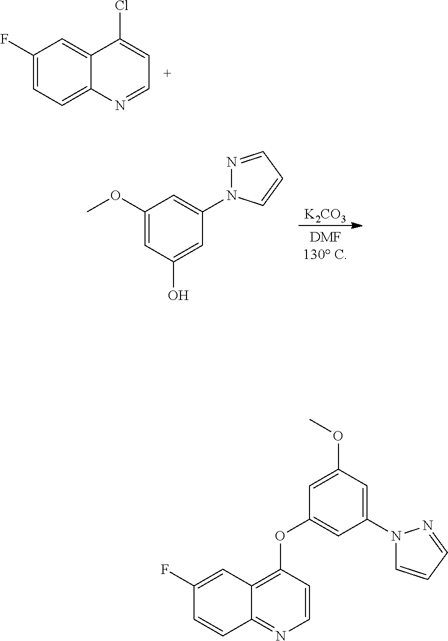

4-chloro-6-fluoroquinoline (72.63 mg, 0.4 mmol, 1.0 eq), 3-methoxy-5-(1H-pyrazol-1-yl)phenol (76.08 mg, 0.4 mmol, 1.0 eq) and K₂CO₃ (110.56 mg, 0.8 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (72.6 mg, yield=54.2%, purity=99%)

TLC R_{f}=0.35 (PE/EA=1/1)

MS (ESI⁺): m/z=336.40 (M+1)

¹H NMR: (400 MHz, DMSO-d6) δ8.72 (d, J=5.1 Hz, 1H), 8.61 (d, J=2.6 Hz, 1H), 8.13 (dd, J=9.3, 5.4 Hz, 1H), 7.97 (dd, J=9.5, 2.9 Hz, 1H), 7.82-7.70 (m, 2H), 7.45 (t, J=2.1 Hz, 1H), 7.42 (t, J=2.0 Hz, 1H), 6.92-6.80 (m, 2H), 6.55 (dd, J=2.6, 1.8 Hz, 1H), 3.87 (s, 3H). ¹³C NMR: (101 MHz, DMSO) δ 162.07, 160.58, 155.98, 151.48, 146.94, 142.34, 141.76, 132.32, 128.71, 120.88, 120.63, 108.67, 105.97, 105.70, 105.47, 104.81, 103.48, 102.03, 56.38.

### Example 19. N-(3-((6-fluoroquinolin-4-yl)oxy)-5-methoxyphenyl)acetamide (Compound 20)

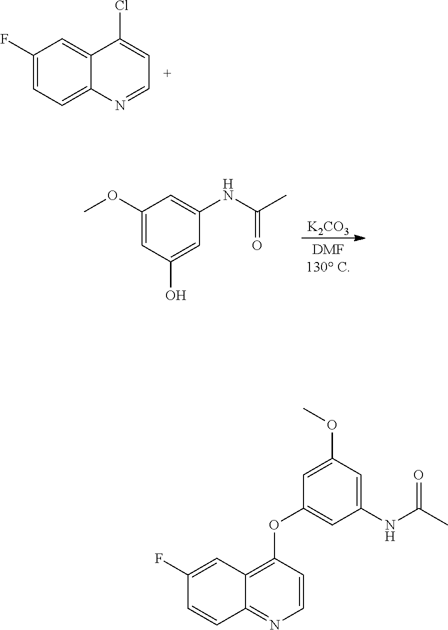

4-chloro-6-fluoroquinoline (72.63 mg, 0.4 mmol, 1.0 eq), N-(3-hydroxy-5-methoxyphenyl)acetamide (72.48 mg, 0.4 mmol, 1.0 eq) and K₂CO₃ (110.56 mg, 0.8 mmol, 2 eq) were added to a round-bottom flask with a magnetic bar, then 2 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (65 mg, yield=49.8%, purity=99%)

TLC R_{f}=0.2 (PE/EA=1/4)

MS (ESI⁺): m/z=327.50 (M+1)

¹H NMR: (400 MHz, DMSO-d6) δ 10.11 (s, 1H), 8.71 (d, J=5.2 Hz, 1H), 8.12 (dd, J=9.3, 5.4 Hz, 1H), 7.93 (dd, J=9.5, 2.9 Hz, 1H), 7.74 (td, J=8.8, 2.9 Hz, 1H), 7.15 (d, J=2.2 Hz, 2H), 6.78 (d, J=5.1 Hz, 1H), 6.61 (t, J=2.2 Hz, 1H), 3.76 (s, 3H), 2.04 (s, 3H). ¹³C NMR: (101 MHz, DMSO) δ 169.13, 161.39, 160.69, 159.00, 155.36, 151.45, 146.93, 142.13, 132.40, 120.87, 120.61, 105.92, 105.46, 103.74, 102.44, 101.65, 55.90, 24.58.

### Example 20. 6-bromo-4-(3-methoxy-5-(1H-pyrazol-1-yl)phenoxy)quinoline (Compound 21)

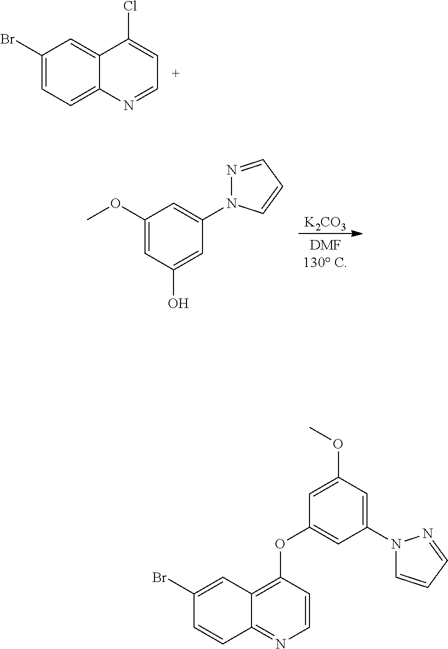

4-bromo-4-chloroquinoline (72.75 mg, 0.3 mmol, 1.0 eq), 3-methoxy-5-(1H-pyrazol-1-yl)phenol (57.06 mg, 0.3 mmol, 1.0 eq) and K₂C₀₃ (82.92 mg, 0.6 mmol, 2.0 eq) were added to a round-bottom flask with a magnetic bar, then 2 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (103.7 mg, yield=87.3%, purity=99%)

TLC R_{f}=0.4 (PE/EA=1/1)

MS (ESI⁺): m/z=398.00 (M+1)

¹H NMR: (400 MHz, DMSO-d6) δ 8.76 (d, J=5.2 Hz, 1H), 8.60 (d, J=2.6 Hz, 1H), 8.46 (d, J=2.1 Hz, 1H), 8.04-7.91 (m, 2H), 7.75 (d, J=1.7 Hz, 1H), 7.44 (dt, J=10.4, 2.0 Hz, 2H), 6.89 (t, J=2.2 Hz, 1H), 6.86 (d, J=5.1 Hz, 1H), 6.55 (t, J=2.1 Hz, 1H), 3.87 (s, 3H). ¹³C NMR: (101 MHz, DMSO) δ 162.07, 160.05, 155.84, 152.68, 148.29, 142.33, 141.79, 133.86, 131.63, 128.72, 124.06, 122.37, 120.02, 108.69, 106.10, 104.87, 103.53, 102.13, 56.40.

### Example 21. N-(3-((6-bromoquinolin-4-yl)oxy)-5-methoxyphenyl)acetamide (Compound 22)

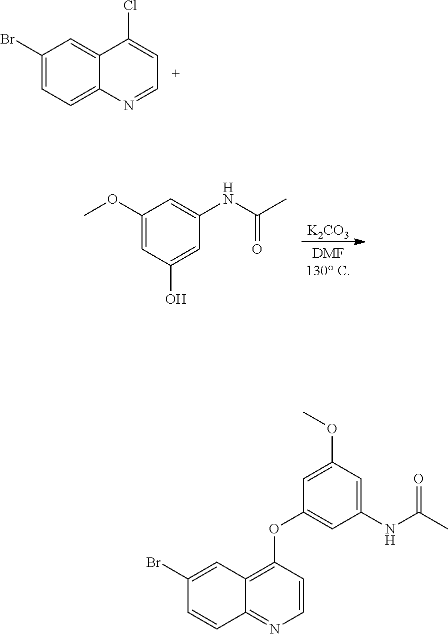

6-bromo-4-chloroquinoline (72.75 mg, 0.3 mmol, 1.0 eq), N-(3-hydroxy-5-methoxyphenyl)acetamide (54.357 mg, 0.3 mmol, 1.0 eq) and K₂CO₃ (110.56 mg, 0.8 mmol, 2 eq) were added to a round-bottom flask with a magnetic bar, then 2 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (68 mg, yield=58.5%, purity=99%)

TLC R_{f}=0.15 (PE/EA=1/4)

MS (ESI⁺): m/z=388.90 (M+1)

¹H NMR: (400 MHz, DMSO-d6) δ 10.12 (s, 1H), 8.75 (d, J=5.1 Hz, 11H), 8.41 (d, J=2.1 Hz, 1H), 8.14-7.74 (m, 2H), 7.16 (d, J=2.2 Hz, 2H), 6.77 (d, J=5.1 Hz, 1H), 6.62 (t, J=2.2 Hz, 1H), 3.76 (s, 3H), 2.04 (s, 3H). ¹³C NMR: (101 MHz, DMSO) δ 169.13, 161.41, 160.13, 155.21, 152.62, 148.27, 142.13, 133.83, 131.62, 124.02, 122.40, 119.99, 106.04, 103.78, 102.54, 101.68, 55.91, 24.60.

### Example 22. 6,7-dimethoxy-4-(3-methoxy-5-(1H-pyrazol-1-yl)phenoxy)quinoline (Compound 26)

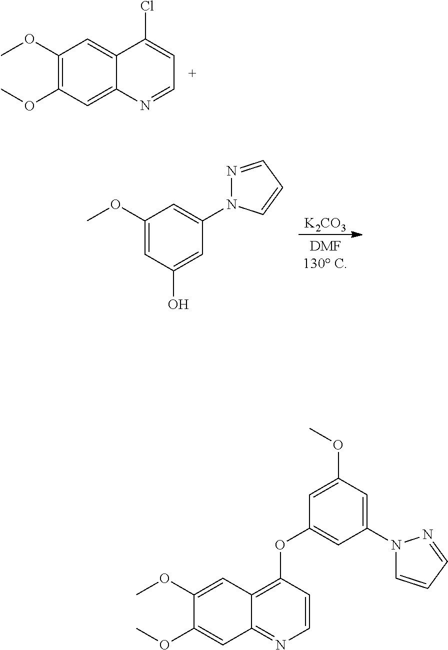

4-chloro-6,7-dimethoxyquinoline (89.464 mg, 0.4 mmol, 1.0 eq), 3-methoxy-5-(1H-pyrazol-1-yl)phenol (76.08 mg, 0.4 mmol, 1.0 eq) and K₂CO₃ (110.56 mg, 0.8 mmol, 2.0 eq) were added to a round-bottom flask with a magnetic bar, then 2 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (49.7 mg, yield=33%, purity=99%)

TLC R_{f}=0.3 (PE/EA=1/2)

MS (ESI⁺): m/z=378.40 (M+1)

¹H NMR: (400 MHz, DMSO-d6) δ 8.62 (d, J=2.5 Hz, 1H), 8.53 (d, J=5.2 Hz, 1H), 7.75 (d, J=1.7 Hz, 1H), 7.51 (s, 1H), 7.42 (d, J=2.4 Hz, 2H), 7.37 (t, J=2.0 Hz, 1H), 6.83 (t, J=2.2 Hz, 1H), 6.68 (d, J=5.2 Hz, 1H), 6.59-6.52 (m, 1H), 3.96 (s, 3H), 3.94 (s, 3H), 3.86 (s, 3H).

### Example 23. N-(3-((6,7-dimethoxyquinolin-4-yl)oxy)-5-methoxyphenyl)acetamide (Compound 27)

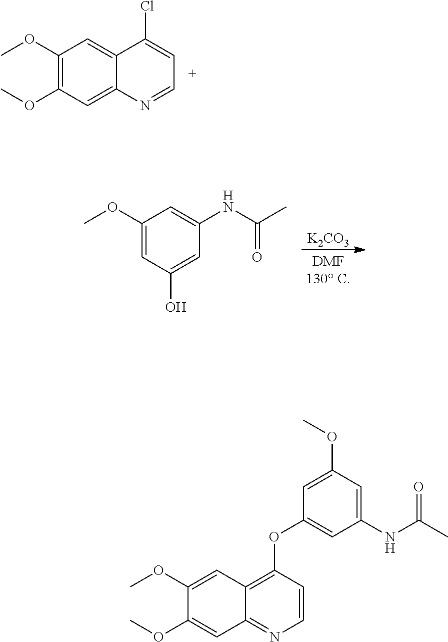

4-chloro-6,7-dimethoxyquinoline (89.44 mg, 0.4 mmol, 1.0 eq), N-(3-hydroxy-5-methoxyphenyl)acetamide (72.476 mg, 0.4 mmol, 1.0 eq) and K₂CO₃ (110.56 mg, 0.8 mmol, 2.0 eq) were added to a round-bottom flask with a magnetic bar, then 2 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (33.4 mg, yield=22.7%, purity=96.7%)

TLC R_{f}=0.1 (PE/EA=1/8)

MS (ESI⁺): m/z=369.50 (M+1)

¹H NMR: (400 MHz, DMSO-d6) δ 10.09 (s, 1H), 8.51 (d, J=5.2 Hz, 1H), 7.47 (s, 11H), 7.41 (s, 1H), 7.12 (p, J=1.8 Hz, 2H), 6.63-6.54 (m, 2H), 3.94 (d, J=8.9 Hz, 6H), 3.76 (s, 3H), 2.03 (s, 3H). ¹³C NMR: (101 MHz, DMSO) δ 169.11, 161.32, 159.68, 155.82, 153.05, 149.86, 149.30, 146.97, 142.02, 115.82, 108.28, 104.47, 103.70, 102.12, 101.59, 99.44, 56.18, 56.13, 55.87, 24.58.

### Example 24. methyl 3-((6,7-dimethoxyquinazolin-4-yl)oxy)-5-methoxybenzoate

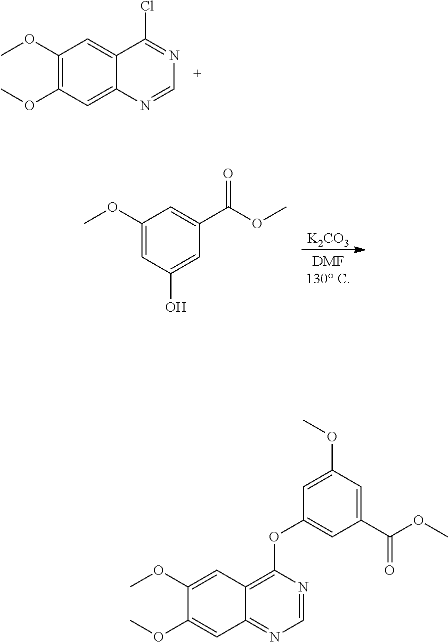

4-chloro-6,7-dimethoxyquinoline (134.8 mg, 0.6 mmol, 1.0 eq), methyl 3-hydroxy-5-methoxybenzoate (109.2 mg, 0.6 mmol, 1.0 eq) and K₂CO₃ (165.6 mg, 0.6 mmol, 1.2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (130 mg, yield=58.56%, purity=98.2%)

TLC R_{f}=0.2 (PE/EA=1/1)

MS (ESI⁺): m/z=371.50 (M+1)

¹H NMR: (400 MHz, DMSO-d6) δ 8.56 (s, 1H), 7.53 (d, J=0.9 Hz, 1H), 7.43 (dd, J=2.1, 1.4 Hz, 1H), 7.39 (dd, J=2.5, 1.3 Hz, 1H), 7.36 (s, 1H), 7.27 (t, J=2.3 Hz, 1H), 3.97 (d, J=8.5 Hz, 6H), 3.84 (d, J=3.6 Hz, 6H). ¹³C NMR: 13C NMR (101 MHz, DMSO) δ 165.32, 164.43, 160.33, 155.76, 153.38, 152.06, 150.04, 148.91, 131.67, 115.11, 113.33, 111.53, 109.61, 106.64, 100.59, 56.12, 55.82, 52.42.

### Example 25. 7-methoxy-4-(3-methoxy-5-(1H-pyrazol-1-yl)phenoxy)quinoline-6-carboxamide (Compound 30)

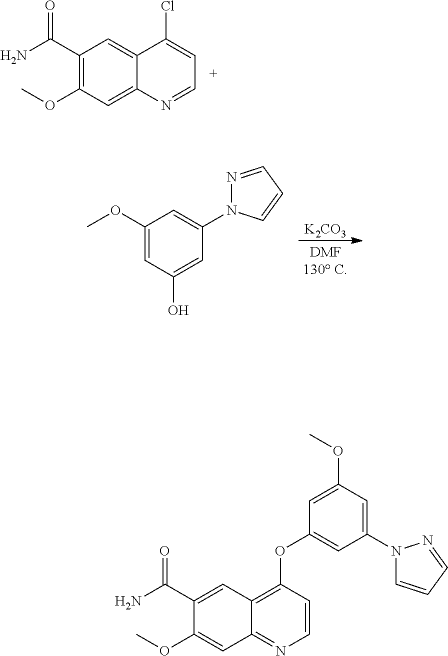

4-chloro-7-methoxyquinoline-6-carboxamide (70.995 mg, 0.3 mmol, 1.0 eq), 3-methoxy-5-(1H-pyrazol-1-yl)phenol (57.06 mg, 0.3 mmol, 1.0 eq) and K₂CO₃ (82.92 mg, 0.6 mmol, 2.0 eq) were added to a round-bottom flask with a magnetic bar, then 2 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (104 mg, yield=88.8%, purity=94.2%)

TLC R_{f}=0.45 (DCM/MeOH=15/1)

MS (ESI⁺): m/z=391.50 (M+1)

¹H NMR: (400 MHz, DMSO-d6) δ 8.71 (t, J=2.6 Hz, 2H), 8.61 (d, J=2.6 Hz, 1H), 7.94-7.84 (m, 1H), 7.82-7.77 (m, 1H), 7.75 (d, J=1.7 Hz, 1H), 7.55 (s, 1H), 7.45 (t, J=2.1 Hz, 1H), 7.41 (t, J=2.0 Hz, 1H), 6.88 (t, J=2.2 Hz, 1H), 6.71 (d, J=5.2 Hz, 1H), 6.55 (t, J=2.2 Hz, 1H), 4.05 (s, 3H), 3.87 (s, 3H). ¹³C NMR: (101 MHz, DMSO) δ 166.23, 162.06, 161.44, 158.52, 155.97, 153.88, 152.18, 142.31, 141.78, 128.74, 125.54, 125.25, 115.07, 108.69, 108.41, 104.78, 104.25, 103.41, 102.01, 56.66, 56.38.

### Example 26. 4-(3-acetamido-5-methoxyphenoxy)-7-methoxyquinoline-6-carboxamide (Compound 31)

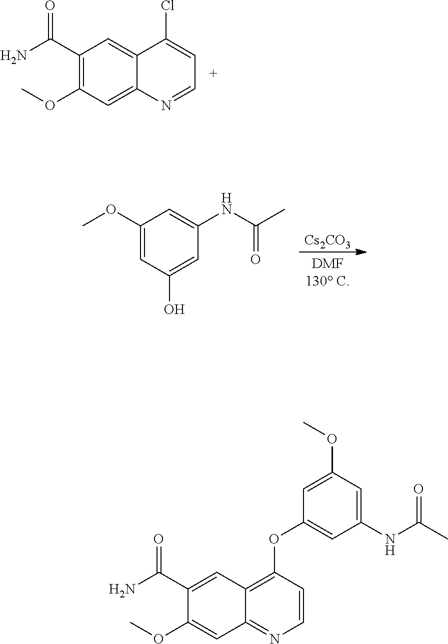

4-chloro-7-methoxyquinoline-6-carboxamide (94.66 mg, 0.4 mmol, 1.0 eq), N-(3-hydroxy-5-methoxyphenyl)acetamide (72.476 mg, 0.4 mmol, 1.0 eq) and K₂CO₃ (261 mg, 0.8 mmol, 2.0 eq) were added to a round-bottom flask with a magnetic bar, then 2 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (57 mg, yield=37.4%, purity=89.3%)

TLC R_{f}=0.1 (DCM/MeOH=30/1)

MS (ESI): m/z=382.60 (M+1)

¹H NMR: (400 MHz, DMSO-d6) δ 10.14 (a, 11H), 8.71 (d, J=5.2 Hz, 1H), 8.65 (a, 11H), 7.96-7.84 (m, 1H), 7.77 (s, 1H), 7.53 (a, 1H), 7.16 (p, J=1.9 Hz, 2H), 6.68-6.58 (m, 2H), 4.04 (s, 3H), 3.76 (s, 3H), 2.04 (s, 3H). ¹³C NMR: (101 MHz, DMSO) δ 169.15, 166.28, 161.59, 161.39, 158.53, 155.35, 153.74, 152.02, 142.10, 125.66, 125.09, 115.10, 108.30, 104.24, 103.66, 102.42, 101.61, 56.65, 55.90, 24.59.

### Example 27. methyl 7-methoxy-4-(3-methoxy-5-(1H-pyrazol-1-yl)phenoxy)quinoline-6-carboxylate (Compound 32)

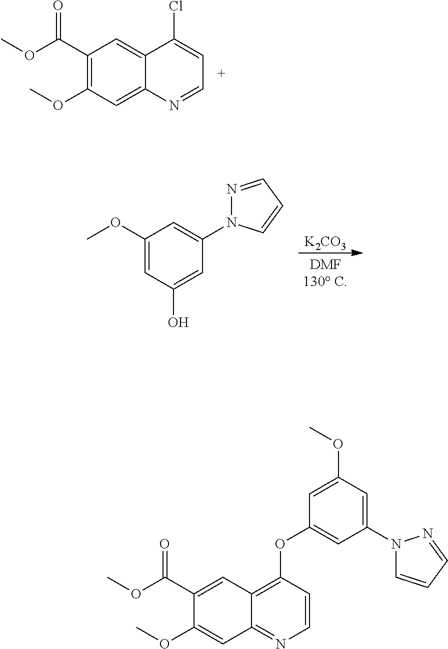

methyl 4-chloro-7-methoxyquinoline-6-carboxylate (75.501 mg, 0.3 mmol, 1.0 eq), 3-methoxy-5-(1H-pyrazol-1-yl)phenol (57.06 mg, 0.3 mmol, 1.0 eq) and K₂CO₃ (82.92 mg, 0.6 mmol, 2.0 eq) were added to a round-bottom flask with a magnetic bar, then 2 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (50.5 mg, yield=41.5%, purity=98.4%)

TLC R_{f}=0.2 (PE/EA=1/1)

MS (ESI⁺): m/z=406.40 (M+1)

¹H NMR: (400 MHz, DMSO-d6) δ 8.73 (d, J=5.3 Hz, 1H), 8.60 (d, J=3.8 Hz, 2H), 7.75 (d, J=1.7 Hz, 1H), 7.55 (s, 1H), 7.44 (dt, J=9.5, 2.0 Hz, 2H), 6.89 (t, J=2.2 Hz, 1H), 6.70 (d, J=5.3 Hz, 1H), 6.55 (dd, J=2.6, 1.8 Hz, 1H), 3.99 (s, 3H), 3.87 (d, J=4.3 Hz, 6H). ¹³C NMR: (101 MHz, DMSO) δ 165.92, 162.06, 161.57, 158.85, 155.73, 154.46, 152.38, 142.31, 141.77, 128.71, 125.63, 122.49, 114.64, 108.78, 108.68, 104.92, 104.15, 103.58, 102.16, 56.61, 56.38, 52.81.

### Example 28. methyl 3-((5H-pyrrolo[3,2-d]pyrimidin-4-yl)oxy)-5-methoxybenzoate (Compound 34)

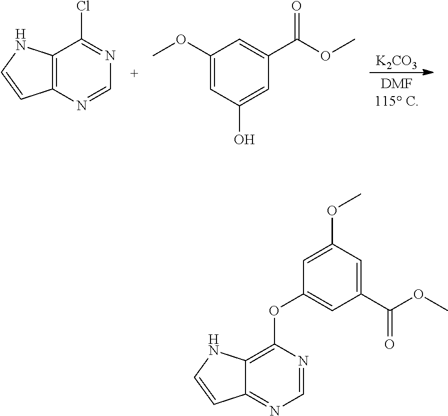

4-chloro-5H-pyrrolo[3,2-d]pyrimidine (91.8 mg, 0.6 mmol, 1.0 eq), methyl 3-hydroxy-5-methoxybenzoate (109.2 mg, 0.6 mmol, 1.2 eq) and K₂CO₃ (165.6 mg, 1.2 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (60 mg, yield=33.5%, purity=82%)

TLC R_{f}=0.2 (PE/EA=1/1)

MS (ESI⁺): m/z=300.40 (M+1)

¹H NMR: (400 MHz, DMSO-d6) δ 12.37 (s, 1H), 8.36 (s, 1H), 7.84 (t, J=3.0 Hz, 1H), 7.42 (ddd, J=9.8, 2.4, 1.5 Hz, 2H), 7.27 (t, J=2.4 Hz, 1H), 6.67 (dd, J=3.1, 1.8 Hz, 1H), 3.86 (s, 3H), 3.85 (s, 3H). SC NMR: (101 MHz, DMSO) δ 165.87, 160.89, 154.79, 153.76, 152.29, 149.12, 132.33, 132.24, 115.44, 114.68, 113.75, 111.75, 102.38, 56.31, 52.93.

### Example 29. 4-(3-methoxy-5-(1H-pyrazol-1-yl)phenoxy)-5H-pyrrolo[3,2-d]pyrimidine (Compound 35)

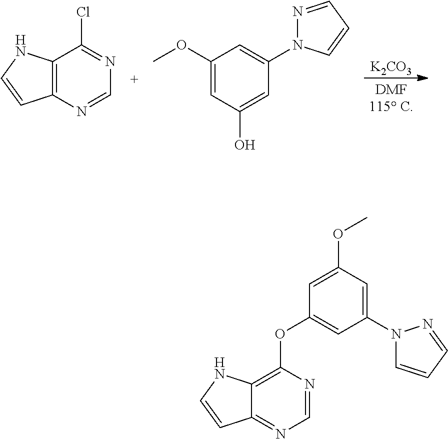

4-chloro-5H-pyrrolo[3,2-d]pyrimidine (76.785 mg, 0.5 mmol, 1.0 eq), 3-methoxy-5-(1H-pyrazol-1-yl)phenol (95.1 mg, 0.5 mmol, 1.2 eq) and K₂CO₃ (138.20 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (59.3 mg, yield=38.6%, purity=91.56%)

TLC R_{f}=0.15 (PE/EA=1/1)

MS (ESI⁺): m/z=308.60 (M+1)

¹H NMR: (400 MHz, DMSO-d6) δ 12.39 (t, J=2.4 Hz, 1H), 8.59 (d, J=2.6 Hz, 1H), 8.38 (s, 1H), 7.84 (t, J=2.9 Hz, 1H), 7.76 (d, J=1.7 Hz, 1H), 7.44 (t, J=2.0 Hz, 1H), 7.42 (t, J=2.1 Hz, 1H), 6.89 (t, J=2.2 Hz, 1H), 6.67 (dd, J=3.0, 1.8 Hz, 1H), 6.55 (dd, J=2.6, 1.7 Hz, 1H), 3.86 (s, 3H). ¹³C NMR: (101 MHz, DMSO) δ 161.48, 154.94, 154.40, 152.25, 149.24, 141.75, 141.67, 132.27, 128.64, 114.74, 108.59, 106.28, 105.01, 102.38, 101.66, 56.31.

### Example 30. dimethyl 5-((5H-pyrrolo[3,2-d]pyrimidin-4-yl)oxy)isophthalate (Compound 36)

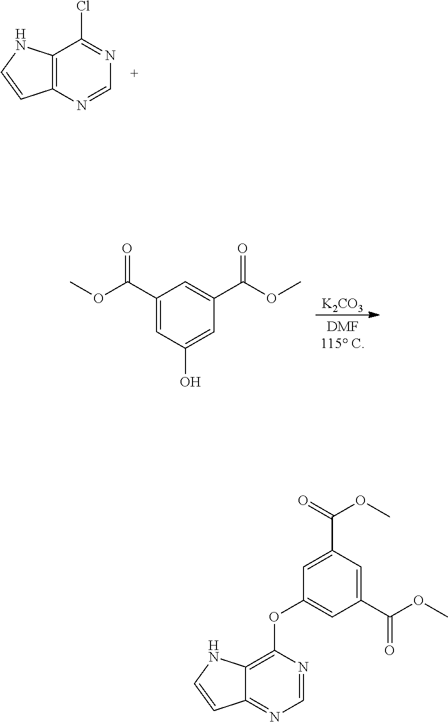

4-chloro-5H-pyrrolo[3,2-d]pyrimidine (153 mg, 1.0 mmol, 1.0 eq), dimethyl 5-hydroxyisophthalate (210 mg, 1.0 mmol, 1.2 eq) and K₂CO₃ (276 mg, 2.0 mmol, 2 eq) were added to a round-bottom flask with a magnetic bar, then 6 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (31 mg, yield=9.5%, purity=96%)

TLC R_{f}=0.33 (PE/EA=3/2)

MS (ESI⁺): m/z=328.50 (M+1)

¹H NMR: (400 MHz, DMSO-d6) δ 12.41 (s, 1H), 8.41 (t, J=1.5 Hz, 1H), 8.36 (s, 1H), 8.14 (d, J=1.5 Hz, 2H), 7.87 (t, J=2.9 Hz, 1H), 6.68 (dd, J=3.1, 1.7 Hz, 1H), 3.91 (s, 6H).

### Example 31. methyl 3-methoxy-5-(thieno[3,2-d]pyrimidin-4-yloxy)benzoate (Compound 37)

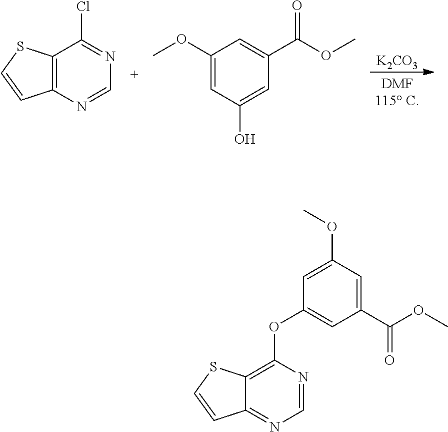

4-chlorothieno[3,2-d]pyrimidine (102.36 mg, 0.6 mmol, 1.0 eq), methyl 3-hydroxy-5-methoxybenzoate (109.2 mg, 0.6 mmol, 1.0 eq) and K₂CO₃ (165.6 mg, 1.2 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (167 mg, yield=88%, purity=97.5%)

TLC R_{f}=0.33 (PE/EA=2/1)

MS (ESI⁺): m/z=317.40 (M+1)

¹H NMR: (400 MHz, DMSO-d6) δ 8.71 (dq, J=2.9, 1.5 Hz, 1H), 8.49 (ddt, J=5.4, 3.9, 1.5 Hz, 1H), 7.68 (ddq, J=6.2, 4.0, 1.9 Hz, 1H), 7.46 (q, J=1.6 Hz, 1H), 7.41 (dq, J=2.6, 1.4 Hz, 1H), 7.30 (dd, J=4.2, 2.2 Hz, 1H), 3.85 (dd, J=4.5, 1.3 Hz, 6H). ¹³C NMR: (101 MHz, DMSO) δ 165.23, 163.30, 163.16, 160.38, 153.98, 152.68, 137.37, 131.78, 124.20, 116.90, 114.96, 113.18, 111.98, 55.86, 52.43.

### Example 32. 4-(3-methoxy-5-(1H-pyrazol-1-yl)phenoxy)thieno[3,2-d]pyrimidine (Compound 38)

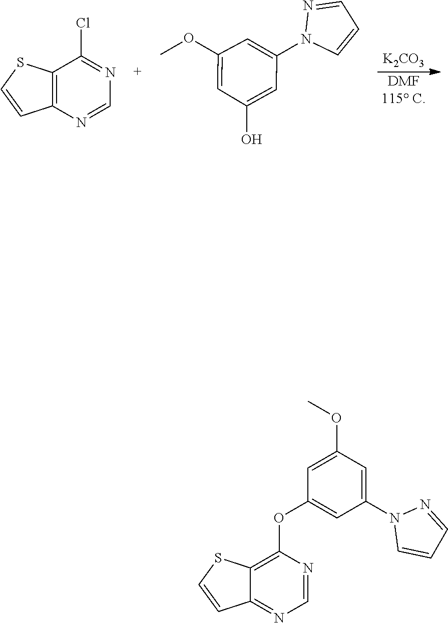

4-chlorothieno[3,2-d]pyrimidine (85.3 mg, 0.5 mmol, 1.0 eq), 3-methoxy-5-(1H-pyrazol-1-yl)phenol (95.1 mg, 0.5 mmol, 1.0 eq) and K₂CO₃ (138.20 mg, 1.0 mmol, 2.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (152.7 mg, yield=94.2%, purity=99%)

TLC R_{f}=0.4 (PE/EA=1/1)

MS (ESI⁺): m/z=325.40 (M+1)

¹H NMR: (400 MHz, DMSO-d6) δ8.75 (s, 1H), 8.58 (d, J=2.5 Hz, 1H), 8.49 (d, J=5.4 Hz, 1H), 7.76 (d, J=1.6 Hz, 1H), 7.70 (d, J=5.4 Hz, 1H), 7.48 (t, J=2.0 Hz, 1H), 7.44 (t, J=2.1 Hz, 1H), 6.94 (t, J=2.2 Hz, 1H), 6.58-6.53 (m, 1H), 3.86 (s, 3H). ¹³C NMR: (101 MHz, DMSO) δ 163.92, 163.71, 161.51, 154.63, 153.84, 141.76, 141.72, 137.91, 128.65, 124.74, 117.40, 108.64, 106.23, 104.99, 102.29, 56.37.

### Example 33. dimethyl 5-(thieno[3,2-d]pyrimidin-4-yloxy)isophthalate (Compound 39)

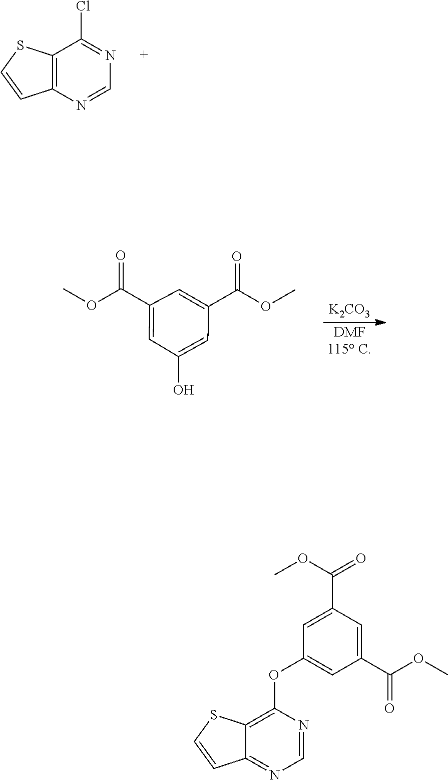

4-chlorothieno[3,2-d]pyrimidine (170 mg, 1.0 mmol, 1.0 eq), dimethyl 5-hydroxyisophthalate (210 mg, 1.0 mmol, 1.0 eq) and K₂CO₃ (276 mg, 2.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (280 mg, yield=81.4%, purity=96%)

TLC R_{f}=0.4 (PF/EA=3/2)

MS (ESI⁺): m/z=345.50 (M+1)

¹H NMR: (400 MHz, DMSO-d6) δ 8.69 (s, 1H), 8.50 (d, J=5.4 Hz, 1H), 8.39 (t, J=1.5 Hz, 1H), 8.15 (d, J=1.5 Hz, 2H), 7.68 (d, J=5.4 Hz, 1H), 3.90 (s, 6H). ¹³C NMR: (101 MHz, DMSO) δ 165.05, 163.73, 163.62, 154.27, 152.43, 138.07, 132.29, 127.79, 127.43, 124.68, 117.49, 53.18.

### Example 34. 7-(3-methoxy-5-(1H-pyrazol-1-yl)phenoxy)thieno[3,2-b]pyridine (Compound 40)

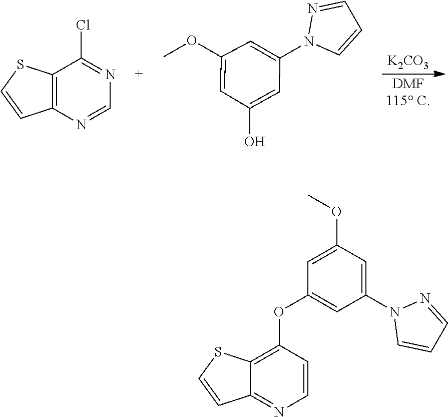

7-chlorothieno[3,2-b]pyridine (84.82 mg, 0.5 mmol, 1.0 eq), 3-methoxy-5-(1H-pyrazol-1-yl)phenol (95.1 mg, 0.5 mmol, 1.0 eq) and K₂CO₃ (138.20 mg, 1.0 mmol, 2.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (31 mg, yield=19.2%, purity=99%)

TLC R_{f}=0.2 (PE/EA=2/1)

MS (ESI⁺): m/z=324.50 (M+1)

¹H NMR: (400 MHz, DMSO-d6) δ 8.61 (d, J=2.6 Hz, 1H), 8.56 (d, J=5.4 Hz, 1H), 8.17 (d, J=5.4 Hz, 1H), 7.75 (d, J=1.7 Hz, 1H), 7.62 (d, J=5.5 Hz, 1H), 7.45 (t, J=2.1 Hz, 1H), 7.41 (t, J=2.0 Hz, 1H), 6.86 (t, J=2.2 Hz, 1H), 6.82 (d, J=5.4 Hz, 1H), 6.55 (dd, J=2.6, 1.7 Hz, 1H), 3.86 (a, 3H). ¹³C NMR: (101 MHz, DMSO) δ 161.99, 159.45, 155.52, 150.04, 142.24, 141.78, 132.56, 128.74, 125.46, 122.23, 108.69, 105.32, 104.67, 103.30, 102.08, 56.40.

### Example 35. methyl 3-methoxy-5-(thieno[2,3-d]pyrimidin-4-yloxy)benzoate (Compound 41)

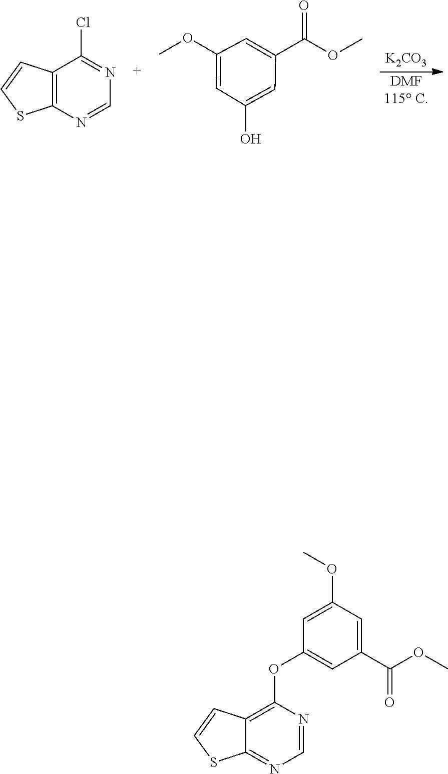

4-chlorothieno[2,3-d]pyrimidine (85.3 mg, 0.5 mmol, 1.0 eq), methyl 3-hydroxy-5-methoxybenzoate (91.1 mg, 0.5 mmol, 1.0 eq) and K₂CO₃ (138 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (83 mg, yield=52.5%, purity=98.7%)

TLC R_{f}=0.4 (PE/EA=4/1)

MS (ESI⁺): m/z=317.50 (M+1)

¹H NMR: (400 MHz, DMSO-d6) δ 8.63 (s, 1H), 7.97 (d, J=5.9 Hz, 1H), 7.65 (d, J=5.9 Hz, 1H), 7.44 (dd, J=2.1, 1.4 Hz, 11H), 7.41 (dd, J=2.5, 1.4 Hz, 1H), 7.28 (t, J=2.3 Hz, 1H), 3.85 (d, J=5.7 Hz, 6H). ¹³C NMR: (101 MHz, DMSO) δ 169.59, 165.77, 163.29, 160.88, 153.49, 153.30, 132.28, 127.98, 119.08, 118.88, 115.46, 113.70, 112.24, 56.33, 52.93.

### Example 36. methyl 4-(3-acetamido-5-methoxyphenoxy)quinoline-6-carboxylate (Compound 42)

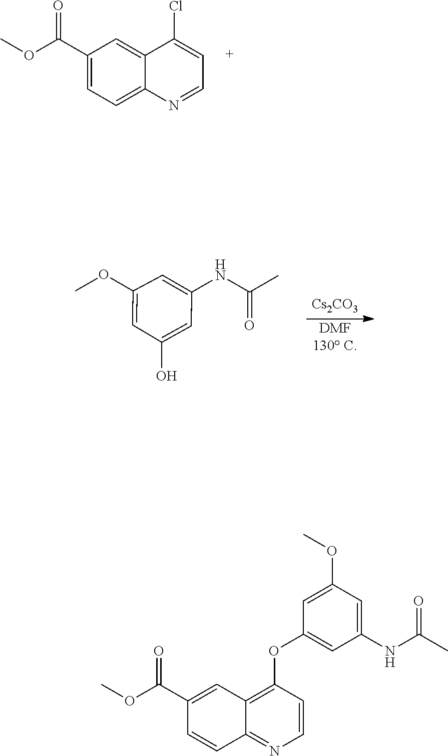

methyl 4-chloroquinoline-6-carboxylate (132.98 mg, 0.6 mmol, 1.2 eq), N-(3-hydroxy-5-methoxyphenyl)acetamide (90.60 mg, 0.5 mmol, 1.0 eq) and Cs₂CO₃ (325.82 mg, 1.0 mmol, 2.0 eq) were added to a round-bottom flask with a magnetic bar, then 2 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a solid. (44.8 mg, yield=24.5%, purity=97.40%)

TLC R_{f}=0.15 (PE/EA=1/3)

MS (ESI⁺): m/z=367.40 (M+1)

¹H NMR: (400 MHz, DMSO-d6) δ 10.13 (s, 1H), 8.89 (d, J=1.9 Hz, 1H), 8.82 (d, J=5.2 Hz, 1H), 8.26 (dd, J=8.8, 2.0 Hz, 1H), 8.12 (d, J=8.8 Hz, 1H), 7.18 (d, J=11.1 Hz, 2H), 6.79 (d, J=5.2 Hz, 1H), 6.66 (d, J=2.4 Hz, 1H), 3.93 (s, 3H), 3.76 (s, 3H), 2.04 (s, 3H). ¹³C NMR: (101 MHz, DMSO) δ 169.14, 166.15, 161.93, 161.45, 155.05, 154.52, 151.34, 142.16, 129.96, 129.62, 127.51, 124.61, 120.42, 105.84, 103.86, 102.68, 101.75, 55.91, 52.93, 24.59.

### Example 37. N-(3-methoxy-5-(thieno[3,2-b]pyridin-7-yloxy)phenyl)acetamide (Compound 43)

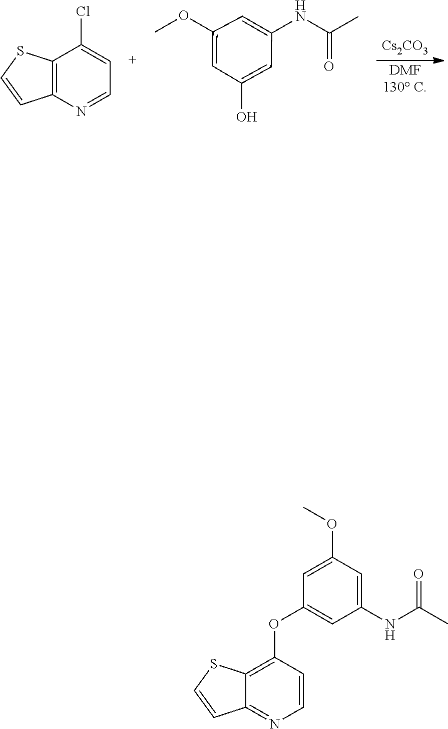

7-chlorothieno[3,2-b]pyridine (101.778 mg, 0.6 mmol, 1.2 eq), N-(3-hydroxy-5-methoxyphenyl)acetamide (90.60 mg, 0.5 mmol, 1.0 eq) and Cs₂CO₃ (325.82 mg, 1.0 mmol, 2.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄, and concentrated in vacuo. The residue was purified by silica gel flash chromatography to afford the product as a white solid. (127.9 mg, yield=81.4%, purity=99%)

TLC R_{f}=0.2 (PE/EA=1/4)

MS (ESI⁺): m/z=315.40 (M+1)

¹H NMR: (400 MHz, DMSO-d6) δ 10.10 (s, 1H), 8.55 (d, J=5.4 Hz, 1H), 8.16 (d, J=5.5 Hz, 1H), 7.61 (d, J=5.4 Hz, 1H), 7.14 (dt, J=15.2, 2.0 Hz, 2H), 6.76 (d, J=5.4 Hz, 1H), 6.61 (t, J=2.3 Hz, 1H), 3.76 (s, 3H), 2.03 (s, 3H). ¹³C NMR: (101 MHz, DMSO) δ 169.14, 161.32, 159.47, 159.41, 154.93, 149.98, 142.01, 132.56, 125.45, 122.28, 105.40, 103.51, 102.46, 101.52, 55.92, 24.59.

### Example 38. 7-Methoxy-4-(3-methoxy-5-(1H-pyrazol-1-yl)phenoxy)-N-methylquinoline-6-carboxamide (Compound 44)

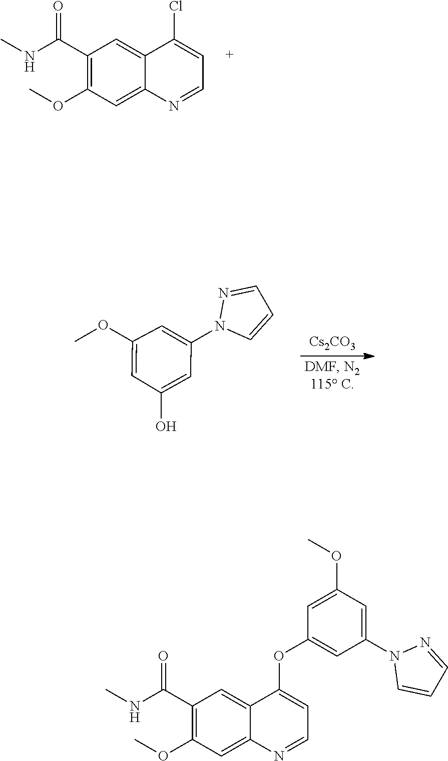

) 4-chloro-7-methoxy-N-methylquinoline-6-carboxamide (125.34 mg, 0.5 mmol, 1.0 eq), 3-methoxy-5-(1H-pyrazol-1-yl)phenol (114.12 mg, 0.6 mmol, 1.2 eq) and Cs₂CO₃ (325.82 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (172.9 mg, yield=85.5%, purity=91.7%)

TLC R_{f}=0.4 (DCM/MeOH=20/1)

MS (ESI⁺): m/z=405.40 (M+1)

¹H NMR: (400 MHz, DMSO-d6) δ8.71 (d, J=5.2 Hz, 1H), 8.65-8.58 (m, 2H), 8.37 (q, J=4.7 Hz, 1H), 7.75 (d, J=1.7 Hz, 1H), 7.55 (s, 1H), 7.44 (d, J=2.2 Hz, 1H), 7.40 (d, J=2.1 Hz, 1H), 6.86 (d, J=2.2 Hz, 1H), 6.72 (d, J=5.2 Hz, 1H), 6.55 (t, J=2.1 Hz, 1H), 4.04 (s, 3H), 3.87 (s, 3H), 2.86 (d, J=4.6 Hz, 3H)

### Example 39. 4-(3-Acetamido-5-methoxyphenoxy)-7-methoxy-N-methylquinoline-6-carboxamide (Compound 45)

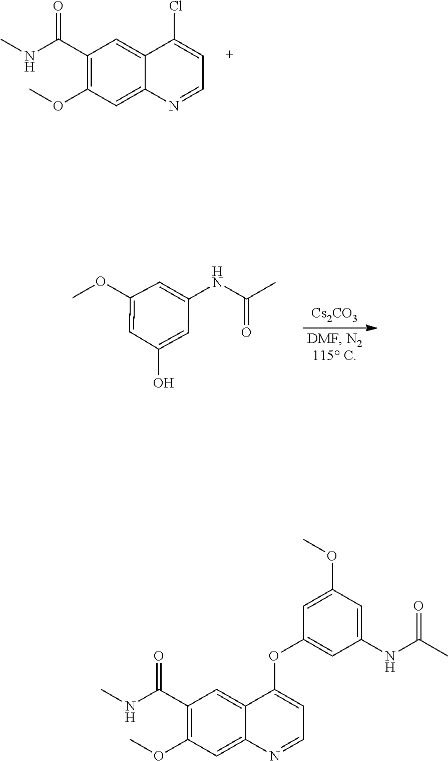

4-chloro-7-methoxy-N-methylquinoline-6-carboxamide (125.34 mg, 0.5 mmol, 1.0 eq), N-(3-hydroxy-5-methoxyphenyl)acetamide (108.71 mg, 0.6 mmol, 12 eq) and Cs₂CO₃ (325.82 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (120.1 mg, yield=61%, purity=98.9%)

TLC R_{f}=0.25 (DCM/MeOH=20/1)

MS (ESI): m/z=396.40 (M+1)

¹H NMR: (400 MHz, DMSO-d6) δ 10.09 (s, 1H), 8.70 (d, J=5.2 Hz, 1H), 8.57 (s, 1H), 8.36 (q, J=4.7 Hz, 1H), 7.53 (s, 1H), 7.14 (dt, J=5.8, 1.9 Hz, 2H), 6.66-6.57 (m, 2H), 4.03 (s, 3H), 3.76 (s, 3H), 2.85 (d, J=4.6 Hz, 3H), 2.03 (s, 3H).

### Example 40. Methyl 4-(3-methoxy-5-(1H-pyrazol-1-yl)phenoxy)quinoline-6-carboxylate (Compound 4)

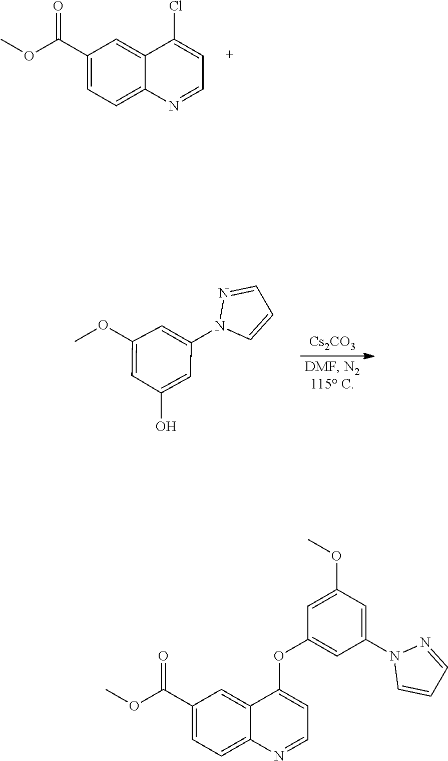

methyl 4-chloroquinoline-6-carboxylate (88.656 mg, 0.4 mmol, 1.0 eq), 3-methoxy-5-(1H-pyrazol-1-yl)phenol (76.08 mg, 0.4 mmol, 1.0 eq) and Cs₂CO₃ (261 mg, 0.8 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (19.8 mg, yield=13.2%, purity=96.43%)

TLC R_{f}=0.35 (PE/EA=1/1)

MS (ESI): m/z=376.30 (M+1)

¹H NMR: (400 MHz, DMSO-d6) δ 8.97 (d, J=1.9 Hz, 1H), 8.85 (d, J=5.2 Hz, 1H), 8.60 (d, J=2.6 Hz, 1H), 8.30 (dd, J=8.8, 2.0 Hz, 1H), 8.16 (d, J=8.9 Hz, 1H), 7.76 (d, J=1.7 Hz, 111), 7.47 (q, J=2.2 Hz, 2H), 6.96-6.87 (m, 2H), 6.56 (t, J=2.1 Hz, 1H), 3.96 (s, 3H), 3.88 (s, 3H)

### Example 41. 4-(3,5-Dimethoxyphenoxy)-6-(furan-2-yl)quinoline (Compound 47)

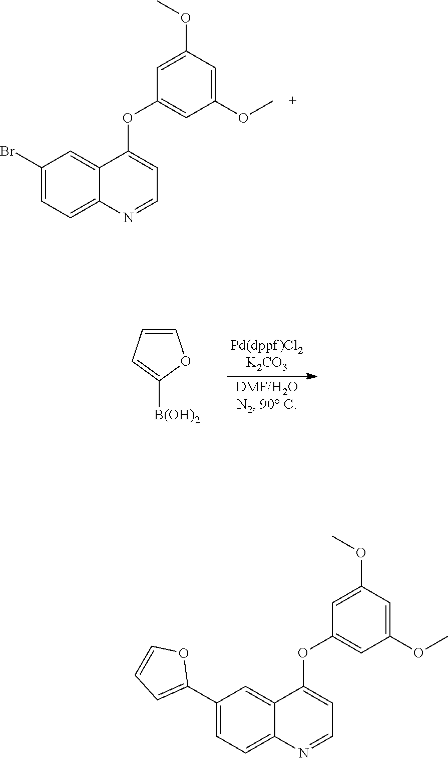 [SMILES: COc1cc(OC)cc(Oc2ccnc3ccc(Br)cc23)c1 ; OB(O)c1ccco1 ; "DMF/H2ON2, 90° C." ; "Pd(dppf)Cl2K2CO3" ; COc1cc(OC)cc(Oc2ccnc3ccc(-c4ccco4)cc23)c1]

6-bromo-4-(3,5-dimethoxyphenoxy)quinoline (216 mg, 0.6 mmol, 1.0 eq), furan-2-ylboronic acid (101 mg, 0.9 mmol, 1.5 eq), Pd(dppf)Cl₂ (11 mg, 0.015 mmol, 2.5% mol) and K₂CO₃ (273 mg, 1.98 mmol, 3.3 eq) were added to a round-bottom flask with a magnetic bar, then DMF (2.65 ml) and H₂O (0.35 ml) (v/v=8/1) were added as solvent. The reaction vessel was evacuated and backfilled with N₂ three times and protected with a balloon of N₂. The reaction mixture was heated at 90° C. for at least 5 h with vigorous stirring. The cooled solution was diluted with ethyl acetate (100 ml) and washed with brine (20 ml, 3 times). The organic phase was dried over anhydrous Na₂SO₄ and concentrated in vacuo. The residue was purified by silica gel flash chromatography to afford the product as a white solid. (220 mg, yield=99%, purity=99%)

TLC R_{f}=0.5 (PE/EA=1/1)

MS (ESI⁺): m/z=348.30 (M+1)

¹H NMR: (400 MHz, DMSO-d6) δ 8.68 (d, J=5.1 Hz, 1H), 8.51 (d, J=2.0 Hz, 1H), 8.18 (dd, J=8.9, 2.0 Hz, 1H), 8.06 (d, J=8.8 Hz, 1H), 7.85 (d, J=1.8 Hz, 1H), 7.21 (d, J=3.4 Hz, 1H), 6.74 (d, J=5.2 Hz, 11H), 6.68 (dd, J=3.5, 1.8 Hz, 1H), 6.59-6.47 (m, 3H), 3.77 (s, 6H).

### Example 42. 6-Bromo-4-(3,5-dimethoxyphenoxy) quinoline (Compound 48)

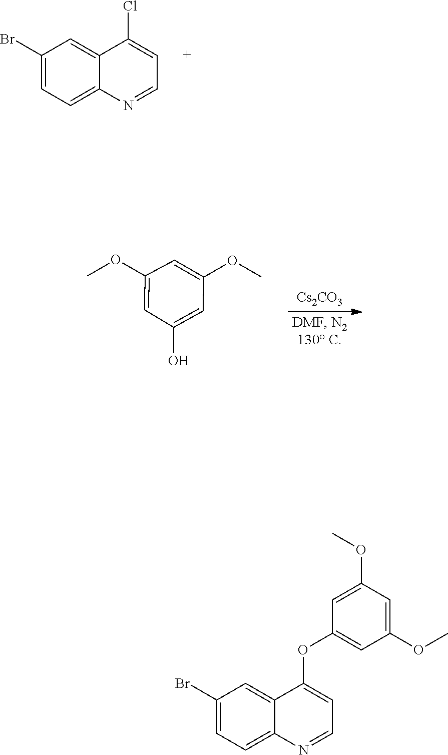

6-bromo-4-chloroquinoline (1.215 g, 5 mmol, 1.0 eq), 3,5-dimethoxyphenol (1 g, 6.5 mmol, 1.3 eq) and Cs₂CO₃ (3.26 g, 10 mmol, 2 eq) were added to a round-bottom flask with a magnetic bar, then 25 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 60 ml ethyl acetate and washed with brine. The organic phase was dried over anhydrous Na₂SO₄ and concentrated in vacuum. The residue was purified by silica gel flash chromatography to afford the product as a white solid. (2.1 g, yield=99%, purity=85.14%)

TLC R_{f}=0.3 (PE/EA=2/1)

MS (ESI): m/z=361.30 (M+1)

### Example 43. 4-(3-Methoxy-5-(1H-pyrazol-1-yl) phenoxy)-6-(trifluoromethyl) quinoline (Compound 49)

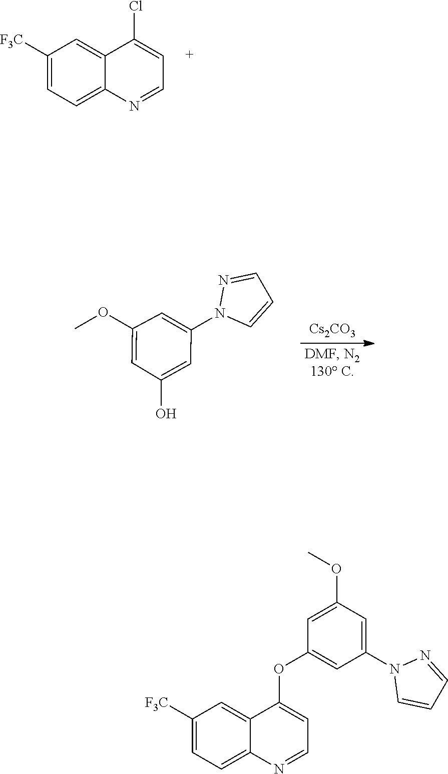

methyl 4-chloroquinoline-6-carboxylate (139 mg, 0.6 mmol, 1.2 eq), 3-methoxy-5-(1H-pyrazol-1-yl) phenol (95 mg, 0.5 mmol, 1.0 eq) and Cs₂CO₃ (326 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (171 mg, yield=88.83%, purity=97.36%)

TLC R_{f}=0.2 (PE/EA=2/1)

MS (ESI⁺): m/z=386.60 (M+1)

¹H NMR: (400 MHz, DMSO-d6) δ8.89 (d, J=5.2 Hz, 1H), 8.67 (s, 1H), 8.60 (d, J=2.5 Hz, 1H), 8.27 (d, J=8.9 Hz, 1H), 8.11 (dd, J=9.0, 2.1 Hz, 1H), 7.76 (d, J=1.7 Hz, 11H), 7.48 (d, J=2.1 Hz, 2H), 6.94 (d, J=5.3 Hz, 2H), 6.56 (t, J=2.1 Hz, 1H), 3.87 (s, 3H).

### Example 44. N-(3-((6-chloroquinolin-4-yl) oxy)-5-methoxyphenyl) acetamide (Compound 50)

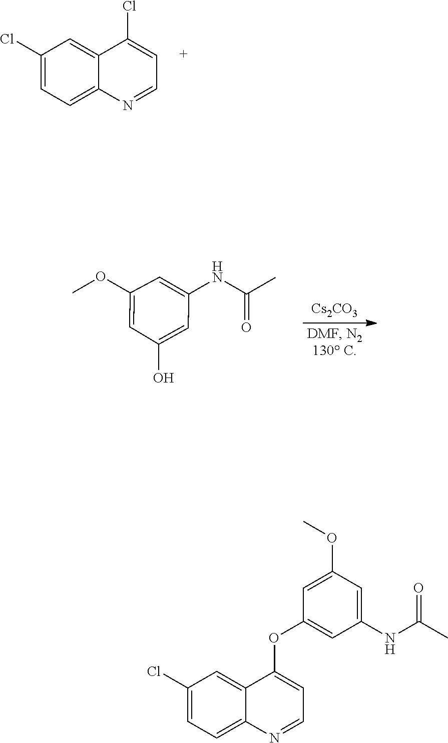

4,6-dichloroquinoline (118.2 mg, 0.6 mmol, 1.2 eq), N-(3-hydroxy-5-methoxyphenyl) acetamide (90.5 mg, 0.5 mmol, 1.0 eq) and Cs₂CO₃ (326 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product a white solid. (124 mg, yield=72.3%, purity=97.99%)

TLC R_{f}=0.25 (PE/EA=1/1)

MS (ESI⁺): m/z=343.50 (M+1)

¹H NMR: (400 MHz, DMSO-d6) δ 10.10 (s, 1H), 8.75 (d, J=5.1 Hz, 1H), 8.26 (d, J=2.4 Hz, 1H), 8.07 (d, J=9.0 Hz, 1H), 7.85 (dd, J=9.1, 2.4 Hz, 1H), 7.15 (d, J=2.4 Hz, 2H), 6.79 (d, J=5.1 Hz, 1H), 6.62 (t, J=2.3 Hz, 1H), 3.76 (s, 3H), 2.04 (s, 3H).

### Example 45. 6-Chloro-4-(3-methoxy-5-(1H-pyrazol-1-yl) phenoxy) quinoline (Compound 51)

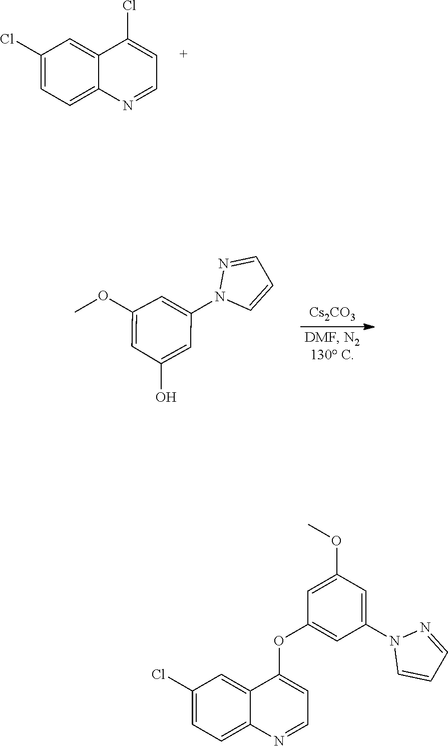

4,6-dichloroquinoline (118.2 mg, 0.6 mmol, 1.2 eq), 3-methoxy-5-(1H-pyrazol-1-yl) phenol (95 mg, 0.5 mmol, 1.0 eq) and Cs₂CO₃ (326 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as an oil. (150 mg, yield=85.4%, purity=97.99%)

TLC R_{f}=0.25 (PE/EA=2/1)

MS (ESI⁺): m/z=352.50 (M+1)

¹H NMR: (400 MHz, DMSO-d6) δ8.76 (d, J=5.2 Hz, 1H), 8.60 (d, J=2.6 Hz, 1H), 8.31 (d, J=2.4 Hz, 1H), 8.09 (d, J=9.0 Hz, 1H), 7.87 (dd, J=9.0, 2.4 Hz, 1H), 7.76 (d, J=1.7 Hz, 1H), 7.44 (dt, J=12.4, 2.0 Hz, 2H), 6.92-6.85 (m, 2H), 6.56 (t, J=2.1 Hz, 1H), 3.87 (s, 3H)

### Example 46. 4-(3-Acetamido-5-methoxyphenoxy) quinoline-6-carboxylic acid (Compound 52)

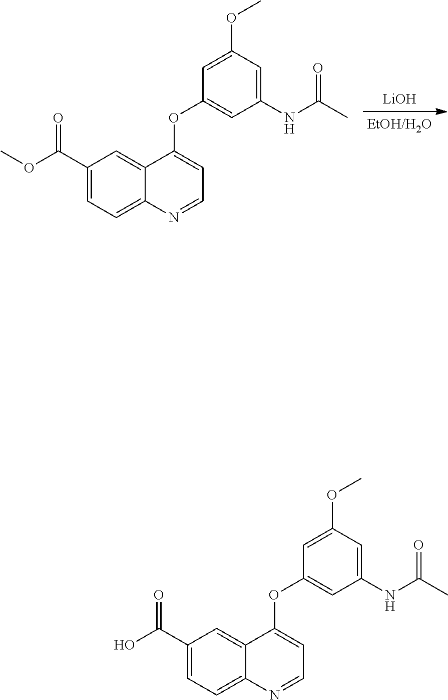

Methyl methyl 4-(3-acetamido-5-methoxyphenoxy) quinoline-6-carboxylate (109.8 mg, 0.3 mmol, 1.0 eq) and LiOH (25.2 mg, 0.6 mmol, 2.0 eq) were added to a round-bottom flask with a magnetic bar. Then 1 ml EtOH and 0.5 ml H₂O were added as solvent. The reaction mixture was stirred overnight. When 4-(3-acetamido-5-methoxyphenoxy) quinoline-6-carboxylate was consumed, the pH of reaction mixture was adjusted to 7 and some white solid formed, which was filtered and dried to give the product. (36 mg, yield=34%, purity=77%)

MS (ESI⁺): m/z=353.50 (M+1)

### Example 47. 4-(3-Acetamido-5-methoxyphenoxy)-N-methylquinoline-6-carboxamide (Compound 53)

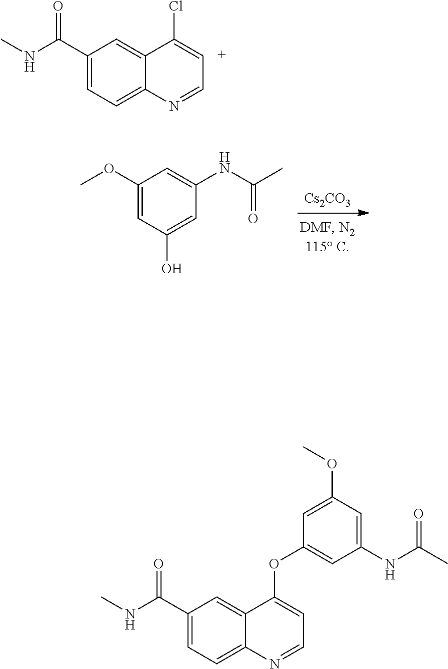

4-chloro-N-methylquinoline-6-carboxamide (44.1 mg, 0.2 mmol, 1.0 eq), N-(3-hydroxy-5-methoxyphenyl) acetamide (36.2 mg, 0.2 mmol, 1.2 eq) and Cs₂CO₃ (130.3 mg, 0.4 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (11 mg, yield=15%, purity=65%)

TLC R_{f}=0.25 (DCM/MeOH=20/1)

MS (ESI⁺): m/z=366.40 (M+1)

### Example 48. 4-(3,5-Dimethoxyphenoxy)-5-methyl-5H-pyrrolo[3,2-d] pyrimidine (Compound 54)

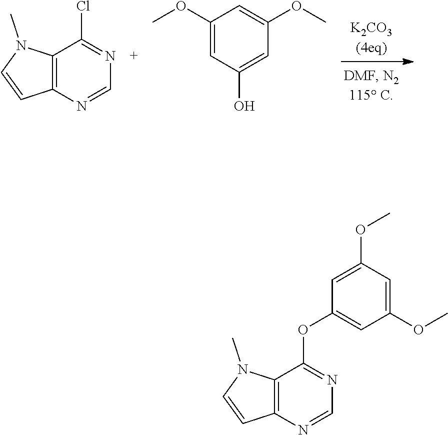

4-chloro-5-methyl-5H-pyrrolo[3,2-d] pyrimidine (83.8 mg, 0.5 mmol, 1.0 eq), 3,5-dimethoxyphenol (77 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (37.8 mg, yield=13.3%, purity=95%)

TLC R_{f}=0.25 (PE/EA=2/1)

MS (ESI⁺): m/z=286.30 (M+1)

¹H NMR: (400 MHz, DMSO-d6) δ 8.31 (s, 1H), 7.78 (d, J=3.0 Hz, 1H), 6.60 (d, J=3.0 Hz, 1H), 6.51 (d, J=2.2 Hz, 2H), 6.45 (d, J=2.3 Hz, 1H), 4.09 (s, 3H), 3.76 (a, 6H).

### Example 49. N-(3-Methoxy-5-((6-(trifluoromethyl)quinolin-4-yl)oxy)phenyl)acetamide (Compound 55)

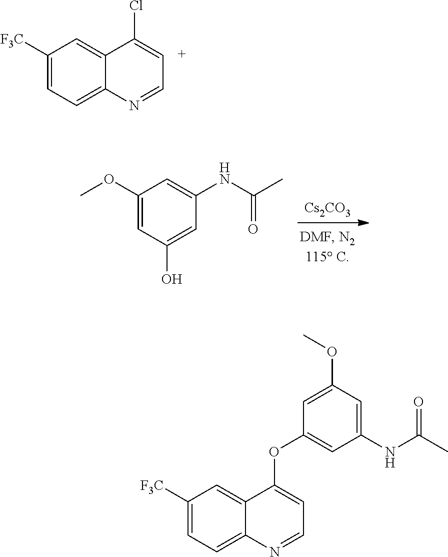

4-chloro-6-(trifluoromethyl)quinoline (139 mg, 0.6 mmol, 1.2 eq), N-(3-hydroxy-5-methoxyphenyl)acetamide (90.5 mg, 0.5 mmol, 1.0 eq) and Cs₂CO₃ (325.82 mg, 1.0 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (91 mg, yield=24.2%, purity=99%)

TLC R_{f}=0.35 (PE/EA=1/2)

MS (ESI⁺): m/z=377.30 (M+1)

¹H NMR: (400 MHz, DMSO-d6) δ 10.12 (s, 1H), 8.87 (d, J=5.2 Hz, 1H), 8.62 (s, 1H), 8.25 (d, J=8.9 Hz, 1H), 8.09 (dd, J=8.9, 2.1 Hz, 1H), 7.21 (t, J=1.9 Hz, 1H), 7.16 (t, J=2.0 Hz, 1H), 6.85 (d, J=5.2 Hz, 1H), 6.67 (t, J=2.3 Hz, 1H), 3.76 (s, 3H), 2.04 (s, 3H).

### Example 50. N-(3-methoxy-5-((5-methyl-5H-pyrrolo[3,2-d]pyrimidin-4-yl)oxy) phenyl) acetamide (Compound 56)

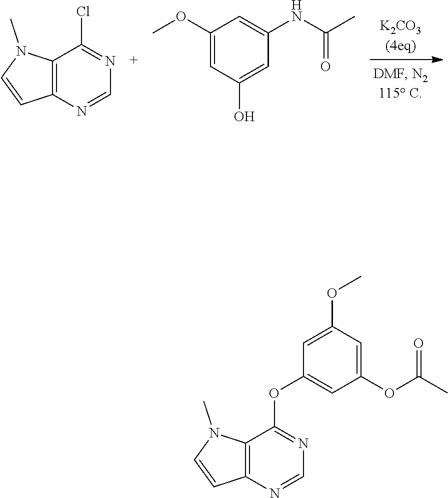 [SMILES: Cn1ccc2ncnc(Cl)c21 ; COc1cc(O)cc(NC(C)=O)c1 ; "K2CO3(4eq)" ; COc1cc(OC(C)=O)cc(Oc2ncnc3ccn(C)c23)c1]

4-chloro-5-methyl-5H-pyrrolo[3,2-d]pyrimidine (83.8 mg, 0.5 mmol, 1.0 eq), N-(3-hydroxy-5-methoxyphenyl)acetamide (90.5 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (6.9 mg, yield=2.2%, purity=94.12%)

TLC R_{f}=0.15 (PE/EA=2/1)

MS (ESI⁺): m/z=313.40 (M+1)

### Example 51. 4-(3-Methoxy-5-(1H-pyrazol-1-yl)phenoxy)-N-methylquinoline-6-carboxamide (Compound 57)

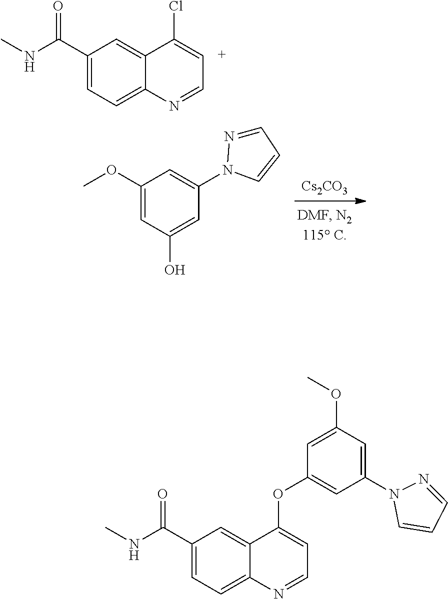

4-chloro-N-methylquinoline-6-carboxamide (110.25 mg, 0.5 mmol, 1.0 eq), 3-methoxy-5-(1H-pyrazol-1-yl)phenol (95 mg, 0.5 mmol, 1.0 eq) and Cs₂CO₃ (326 mg, 1 mmol, 2 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (132.7 mg, yield=70.88%, purity=97.81%)

TLC R_{f}=0.25 (DCM/MeOH=20/1)

MS (ESI⁺): m/z=375.50 (M+1)

¹H NMR: (400 MHz, DMSO-d6) δ8.88 (d, J=2.0 Hz, 1H), 8.81 (dd, J=10.9, 4.9 Hz, 2H), 8.62 (d, J=2.6 Hz, 1H), 8.26 (dd, J=8.8, 2.0 Hz, 1H), 8.11 (d, J=8.8 Hz, 1H), 7.76 (d, J=1.7 Hz, 1H), 7.46 (dt, J=10.1, 2.1 Hz, 2H), 6.93-6.84 (m, 2H), 6.56 (t, J=2.2 Hz, 1H), 3.88 (s, 3H), 2.86 (d, J=4.4 Hz, 3H).

### Example 52. 4-(3-acetamido-5-methoxyphenoxy)-N, N-dimethylquinoline-6-carboxamide (Compound 58)

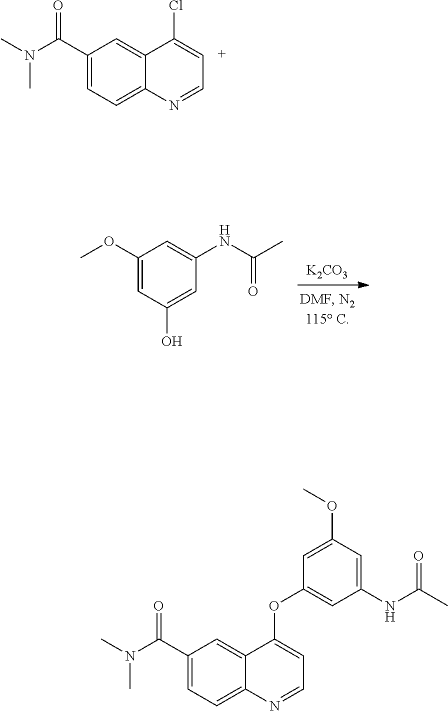

4-chloro-N, N-dimethylquinoline-6-carboxamide (117.34 mg, 0.5 mmol, 1.0 eq), N-(3-hydroxy-5-methoxyphenyl) acetamide (90.5 mg, 0.5 mmol, 1.0 eq) and K₂CO₃ (276 mg, 2.0 mmol, 4 eq) were added to around-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (124.5 mg, yield=65.6%, purity=83%)

TLC R_{f}=0.15 (PE/EA=2/1)

MS (ESI⁺): m/z=380.30 (M+1)

### Example 53. 4-(3-ethoxy-5-(1H-pyrazol-1-yl)phenoxy)-7-methoxyquinoline-6-carboxamide (Compound 59)

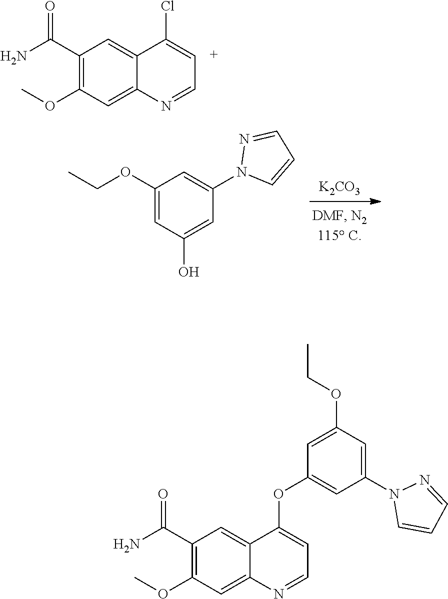

4-chloro-7-methoxyquinoline-6-carboxamide (119 mg, 0.5 mmol, 1.0 eq), 3-ethoxy-5-(1H-pyrazol-1-yl)phenol (102 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (135 mg, yield=66.83%, purity=90.27%)

TLC R_{f}=0.4 (DCM/MeOH=20/1)

MS (ESI⁺): m/z=405.40 (M+1)

¹H NMR: (400 MHz, DMSO-d6) δ 8.74-8.67 (m, 2H), 8.61 (d, J=2.6 Hz, 1H), 7.86 (s, 1H), 7.75 (d, J=1.7 Hz, 2H), 7.55 (s, 1H), 7.43 (d, J=2.1 Hz, 1H), 7.39 (d, J=2.1 Hz, 1H), 6.84 (t, J=2.2 Hz, 1H), 6.71 (d, J=5.2 Hz, 1H), 6.55 (t, J=2.2 Hz, 1H), 4.15 (q, J=6.9 Hz, 2H), 4.05 (s, 3H), 1.36 (t, J=6.9 Hz, 3H)

### Example 54. N-(3-((6-iodoquinolin-4-yl) oxy)-5-methoxyphenyl) acetamide (Compound 60)

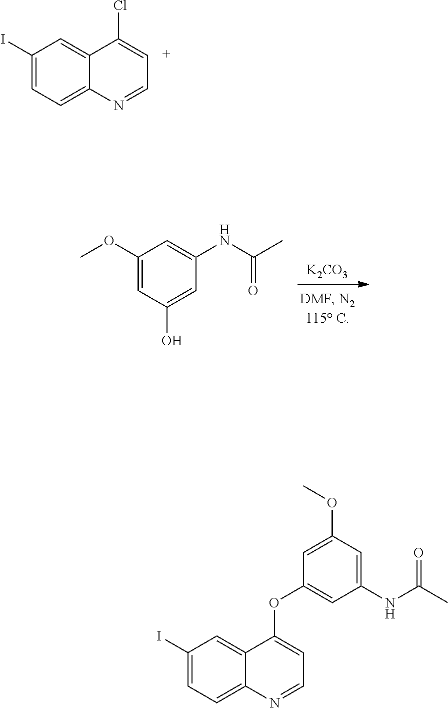

4-chloro-6-iodoquinoline (173.7 mg, 0.6 mmol, 1.2 eq), N-(3-hydroxy-5-methoxyphenyl) acetamide (90.5 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (191.4 mg, yield=88.15%, purity=99%)

TLC R_{f}=0.35 (PE/EA=1/2)

MS (ESI⁺): m/z=435.30 (M+1)

¹H NMR: (400 MHz, DMSO-d6) δ 10.10 (s, 1H), 8.74 (d, J=5.2 Hz, 11H), 8.62 (d, J=1.9 Hz, 1H), 8.09 (dd, J=8.8, 2.0 Hz, 1H), 7.82 (d, J=8.9 Hz, 1H), 7.15 (dt, J=7.0, 2.0 Hz, 2H), 6.76 (d, J=5.2 Hz, 1H), 6.62 (t, J=2.2 Hz, 1H), 3.76 (s, 3H), 2.04 (s, 3H).

### Example 55. 4-(3-Methoxy-5-(1H-pyrazol-1-yl) phenoxy) quinolin-6-carbazamide (Compound 61)

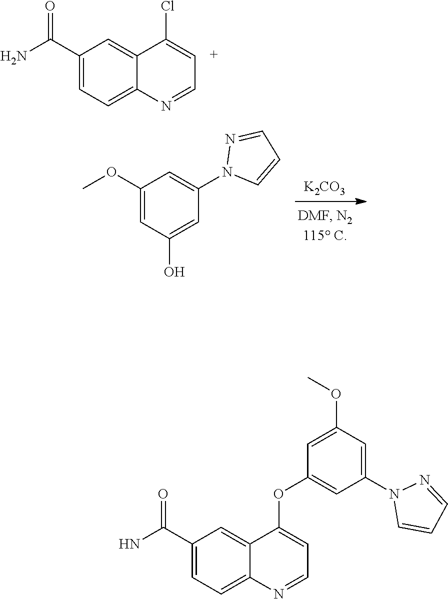

4-chloroquinoline-6-carboxamide (103.3 mg, 0.5 mmol, 1.0 eq), 3-methoxy-5-(1H-pyrazol-1-yl) phenol (95 mg, 0.5 mmol, 1.0 eq) and K₂CO₃ (276 mg, 2 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (117.1 mg, yield=65%, purity=99%)

TLC R_{f}=0.25 (DCM/MeOH=20/1)

MS (ESI⁺): m/z=361.40 (M+1)

### Example 56. 7-Methoxy-4-(3-methoxy-5-(pyridimin-2-yl) phenoxy) quinoline-6-carboxamide (Compound 62)

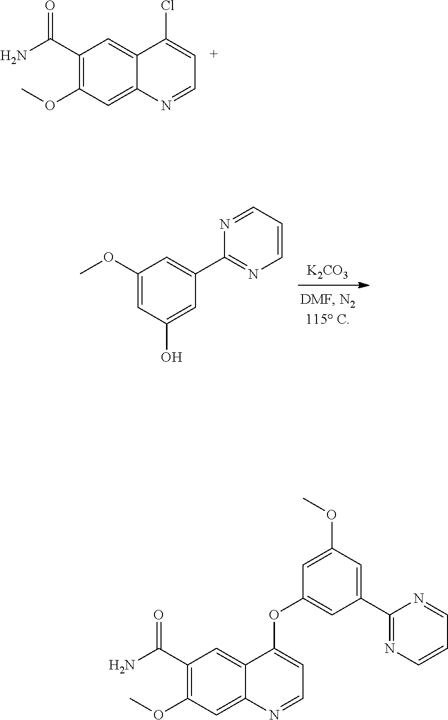

4-chloro-7-methoxyquinoline-6-carboxamide (119 mg, 0.5 mmol, 1.0 eq), 3-methoxy-5-(pyrimidin-2-yl) phenol (101 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (148 mg, yield=73.36%, purity=98.43%)

TLC R_{f}=0.4 (DCM/MeOH=20/1)

MS (ESI⁺): m/z=403.50 (M+1)

¹H NMR: (400 MHz, DMSO-d6) δ 8.91 (d, J=4.8 Hz, 2H), 8.74-8.68 (m, 2H), 7.92 (t, J=1.8 Hz, 1H), 7.87 (s, 1H), 7.80-7.72 (m, 2H), 7.55 (s, 1H), 7.49 (t, J=4.9 Hz, 1H), 7.13 (t, J=2.3 Hz, 1H), 6.70 (d, J=5.2 Hz, 1H), 4.05 (s, 3H), 3.90 (s, 3H).

### Example 57. 6,7-Dimethoxy-4-(3-methoxy-5-(pyrimidin-2-yl) phenoxy) quinoline (Compound 63)

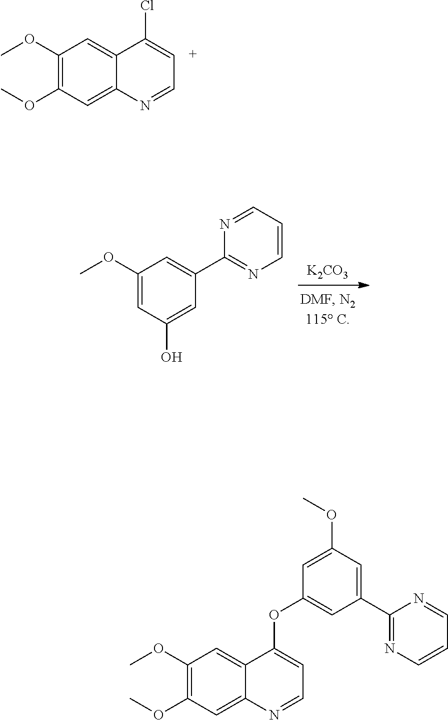

4-4-chloro-6,7-dimethoxyquinoline (119 mg, 0.5 mmol, 1.0 eq), 3-methoxy-5-(pyrimidin-2-yl) phenol (112.5 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (89 mg, yield=45.7%, purity=99%)

TLC R_{f}=0.2 (PE/EA=1/1)

MS (ESI): m/z=390.50 (M+1)

¹H NMR: (400 MHz, DMSO-d6) δ 8.91 (d, J=4.9 Hz, 2H), 8.53 (d, J=5.2 Hz, 1H), 7.90 (dd, J=2.5, 1.3 Hz, 1H), 7.77-7.72 (m, 1H), 7.55-7.45 (m, 2H), 7.43 (s, 1H), 7.10 (t, J=2.3 Hz, 11H), 6.68 (d, J=5.2 Hz, 1H), 3.95 (d, J=11.0 Hz, 6H), 3.90 (a, 3H).

### Example 58. 7-Methoxy-4-(3-methoxy-5-(3-methyl-1,2,4-oxadiazol-5-yl)phenoxy)quinoline-6-carboxamide (Compound 64)

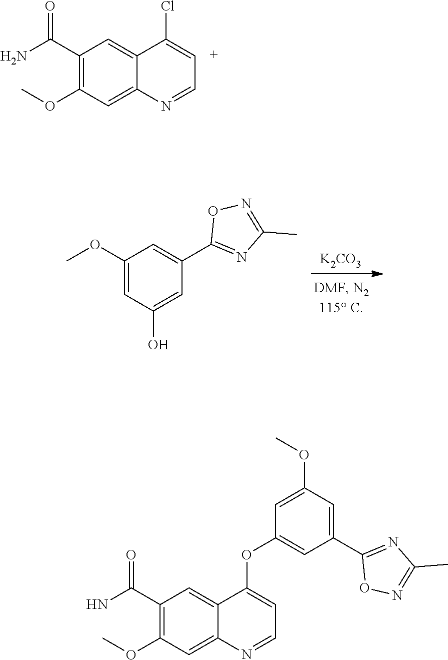

4-chloro-7-methoxyquinoline-6-carboxamide (119 mg, 0.5 mmol, 1.0 eq), 3-methoxy-5-(3-methyl-1,2,4-oxadiazol-5-yl)phenol (103 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (182 mg, yield=89.6%, purity=89%)

TLC R_{f}=0.2 (DCM/MeOH=16/1)

MS (ESI⁺): m/z=407.50 (M+1)

¹H NMR: (400 MHz, DMSO-d6) δ 8.72 (d, J=5.2 Hz, 1H), 8.68 (s, 1H), 7.86 (s, 1H), 7.75 (s, 1H), 7.55 (d, J=4.0 Hz, 2H), 7.49 (t, J=1.8 Hz, 1H), 7.29 (t, J=2.3 Hz, 1H), 6.72 (d, J=5.2 Hz, 1H), 4.05 (s, 3H), 3.91 (s, 3H), 2.41 (s, 3H).

### Example 59. 4-(3-Ethoxy-5-(MH-pyrazol-1-yl) phenoxy) quinoline-6-carboxamide (Compound 65)

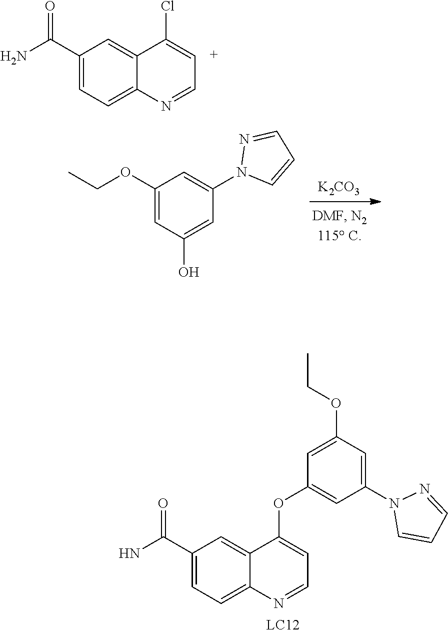

4-chloroquinoline-6-carboxamide (103.3 mg, 0.5 mmol, 1.0 eq), 3-ethoxy-5-(1H-pyrazol-1-yl) phenol (102.1 mg, 0.5 mmol, 1.0 eq) and K₂CO₃ (276 mg, 2 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (108.6 mg, yield=58%, purity=91.47%)

TLC R_{f}=0.2 (DCM/MeOH=20/1)

MS (ESI⁺): m/z=375.40 (M+1)

¹H NMR: (400 MHz, DMSO-d6) δ8.93 (d, J=1.9 Hz, 1H), 8.80 (d, J=5.2 Hz, 1H), 8.61 (d, J=2.6 Hz, 11H), 8.35 (s, 11H), 8.29 (dd, J=8.8, 2.0 Hz, 11H), 8.10 (d, J=8.8 Hz, 11H), 7.75 (d, J=1.7 Hz, 1H), 7.57 (s, 11H), 7.44 (dt, J=10.1, 2.1 Hz, 2H), 6.90-6.84 (m, 2H), 6.55 (t, J=2.1 Hz, 11H), 4.15 (q, J=6.9 Hz, 2H), 1.37 (t, J=7.0 Hz, 3H).

### Example 60. 7-Methoxy-4-(3-methoxy-5-(1-methyl-H-pyrazol-4-yl)phenoxy)quinoline-6-carboxamide (Compound 66)

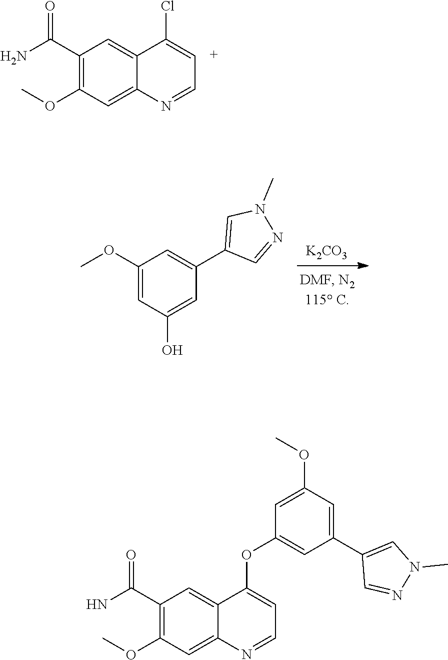

4-chloro-7-methoxyquinoline-6-carboxamide (119 mg, 0.5 mmol, 1.0 eq), 3-methoxy-5-(1-methyl-1H-pyrazol-4-yl)phenol (122.4 mg, 0.6 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (189 mg, yield=93.6%, purity=99%)

TLC R_{f}=0.25 (DCM/MeOH=16/1)

MS (ESI⁺): m/z=405.50 (M+1)

¹H NMR: (400 MHz, DMSO-d6) δ 8.74-8.66 (m, 2H), 8.23 (s, 1H), 7.94 (s, 1H), 7.86 (s, 1H), 7.74 (s, 1H), 7.54 (s, 1H), 7.13 (dt, J=11.2, 1.8 Hz, 2H), 6.74 (d, J=2.3 Hz, 1H), 6.62 (d, J=5.2 Hz, 1H), 4.05 (s, 3H), 3.84 (d, J=7.2 Hz, 6H).

### Example 61. 5-(3-((6-Bromoquinolin-4-yl)oxy)-5-methoyphenyl)-3-methyl-1,2,4-oxadiazole (Compound 67)

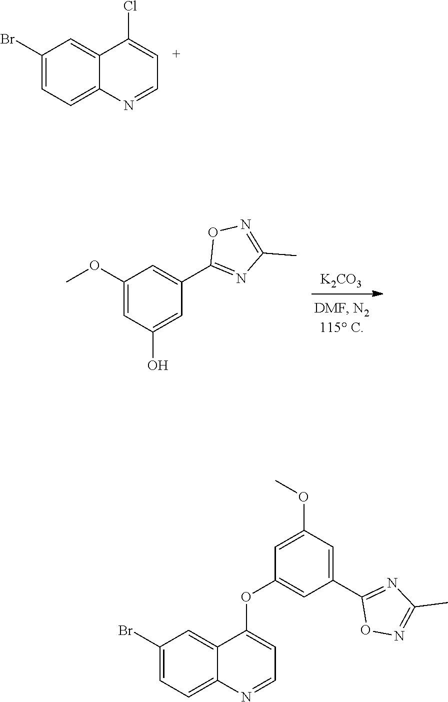

6-bromo-4-chloroquinoline (144 mg, 0.6 mmol, 1.2 eq), 3-methoxy-5-(3-methyl-1,2,4-oxadiazol-5-yl)phenol (103 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (86.1 mg, yield=41.8%, purity=99%)

TLC R_{f}=0.2 (PE/EA=3/1)

MS (ESI⁺): m/z=412.30 (M+1)

¹H NMR: (400 MHz, DMSO-d6) δ 8.78 (d, J=5.2 Hz, 1H), 8.46 (d, J=2.1 Hz, 1H), 8.05-7.94 (m, 2H), 7.55 (dt, J=10.3, 1.7 Hz, 2H), 7.31 (t, J=2.3 Hz, 1H), 6.87 (d, J=5.1 Hz, 1H), 3.91 (s, 3H), 2.42 (s, 3H)

### Example 62. 5-(3-((6-Bromoquinolin-4-yl)oxy)-5-methoxyphenyl)-3-methyl-1,2,4-oxadiazole (Compound 68)

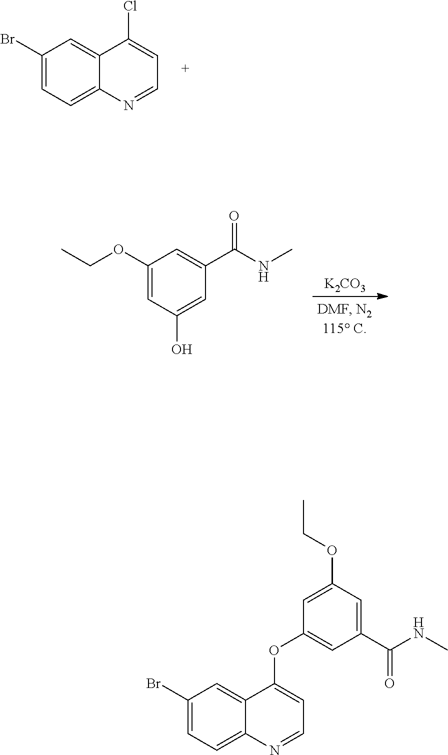

6-bromo-4-chloroquinoline (144 mg, 0.6 mmol, 1.2 eq), 3-methoxy-5-(3-methyl-1,2,4-oxadiazol-5-yl)phenol (103 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (18 mg, yield=10.69%, purity=99%)

TLC R_{f}=0.2 (PE/EA=3/1)

MS (ESI⁺): m/z=401.30 (M+1)

¹H NMR: (400 MHz, DMSO-d6) δ 8.76 (d, J=5.1 Hz, 1H), 8.47 (dd, J=15.3, 3.4 Hz, 2H), 8.05-7.94 (m, 2H), 7.39 (t, J=1.7 Hz, 1H), 7.29 (t, J=1.6 Hz, 1H), 7.10 (d, J=2.3 Hz, 1H), 6.77 (d, J=5.2 Hz, 111), 4.12 (q, J=7.0 Hz, 2H), 2.77 (d, J=4.4 Hz, 3H), 1.35 (t, J=6.9 Hz, 3H).

### Example 63. 4-(3,5-Dimethoxyphenoxy)-7-methoxyquinolin-6-carboxamide (Compound 69)

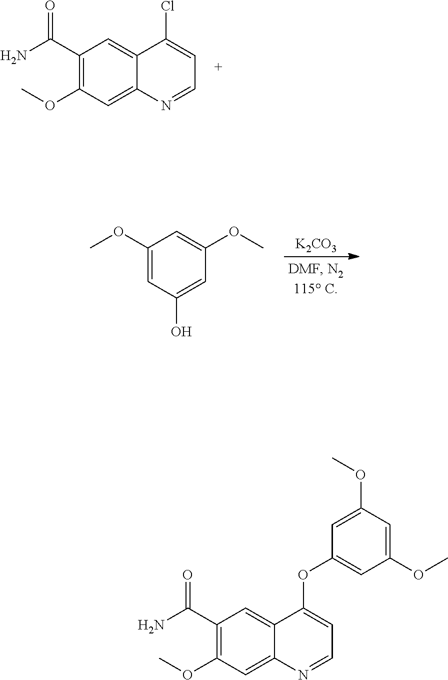

4-chloro-7-methoxyquinoline-6-carboxamide (119 mg, 0.5 mmol, 1.0 eq), 3,5-dimethoxyphenol (92.4 mg, 0.6 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (124 mg, yield=70%, purity=99%)

TLC R_{f}=0.25 (DCM/MeOH=16/1)

MS (ESI⁺): m/z=355.40 (M+1)

¹H NMR: (400 MHz, DMSO-d6) δ 8.72-8.65 (m, 2H), 7.85 (s, 1H), 7.74 (s, 1H), 7.53 (s, 1H), 6.62 (d, J=5.2 Hz, 1H), 6.49 (s, 3H), 4.04 (s, 3H), 3.77 (s, 6H).

### Example 64. 3-Methoxy-N-methyl-5-((6-(trifluoromethyl)quinolin-4-yl) oxy) benzamide (Compound 70)

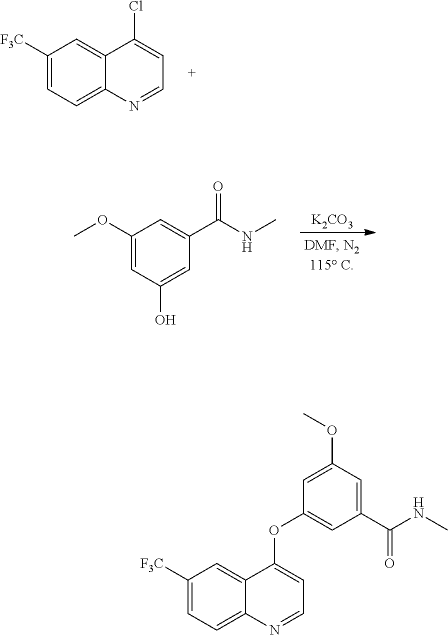

4-chloro-6-(trifluoromethyl) quinoline (139 mg, 0.6 mmol, 1.2 eq), 3-hydroxy-5-methoxy-N-methylbenzamide (90.5 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (144 mg, yield=76.5%, purity=99%)

TLC R_{f}=0.2 (PE/EA=1/2)

MS (ESI⁺): m/z=377.30 (M+1)

¹H NMR: (400 MHz, DMSO-d6) δ 8.88 (d, J=5.2 Hz, 1H), 8.65 (s, 1H), 8.51 (q, J=4.6 Hz, 1H), 8.27 (d, J=8.8 Hz, 1H), 8.11 (dd, J=8.9, 2.1 Hz, 1H), 7.42 (t, J=1.8 Hz, 1H), 7.36 (t, J=1.8 Hz, 1H), 7.18 (t, J=2.3 Hz, 1H), 6.84 (d, J=5.2 Hz, 1H), 3.85 (s, 3H), 2.78 (d, J=4.5 Hz, 3H).

### Example 65. 7-Methoxy-4-(3-methoxy-5-(methylcarbamoyl) phenoxy) quinoline-6-carboxamide (Compound 71)

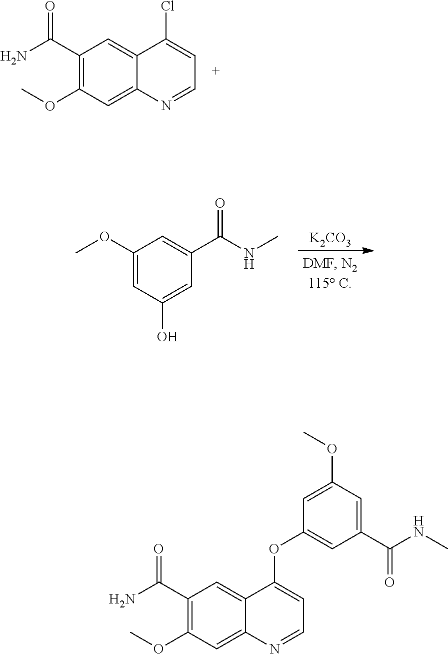

4-chloro-7-methoxyquinoline-6-carboxamide (119 mg, 0.5 mmol, 1.0 eq), 3-hydroxy-5-methoxy-N-methylbenzamide (90.5 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (150 mg, yield=78.5%, purity=99%6)

TLC R_{f}=0.25 (DCM/MeOH=16/1)

MS (ESI⁺): m/z=382.20 (M+1)

¹H NMR: (400 MHz, DMSO-d6) δ 8.74-8.66 (m, 2H), 8.50 (q, J=4.6 Hz, 1H), 7.86 (s, 1H), 7.74 (s, 1H), 7.55 (s, 1H), 7.39 (t, J=1.8 Hz, 1H), 7.29 (t, J=1.7 Hz, 1H), 7.10 (t, J=2.3 Hz, 1H), 6.62 (d, J=5.2 Hz, 1H), 4.05 (s, 3H), 3.85 (s, 3H), 2.77 (d, J=4.5 Hz, 3H).

### Example 66. 3-((6-Bromoquinolin-4-yl) oxy)-5-methoxy-N-methylbenzamide (Compound 72)

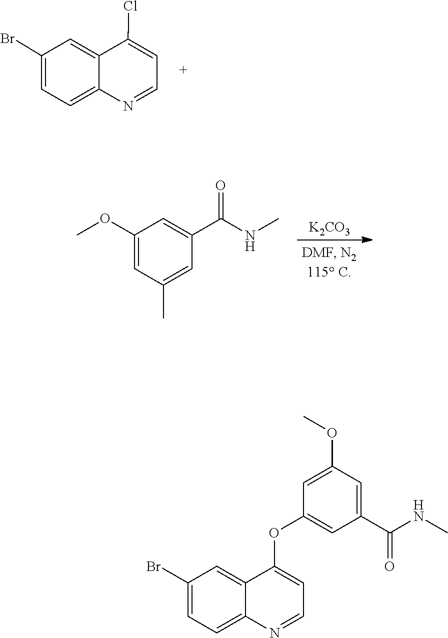

6-bromo-4-chloroquinoline (145.5 mg, 0.6 mmol, 1.0 eq), 3-hydroxy-5-methoxy-N-methylbenzamide (90.5 mg, 0.5 mmol, 1.0 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (164 mg, yield=85%, purity=99%)

TLC R_{f}=0.15 (PE/EA=1/4)

MS (ESI⁺): m/z=387.10 (M+1)

¹H NMR: (400 MHz, DMSO-d6) δ8.76 (d, J=5.2 Hz, 1H), 8.50 (q, J=4.5 Hz, 1H), 8.45 (d, J=2.1 Hz, 1H), 8.05-7.93 (m, 2H), 7.40 (t, J=1.8 Hz, 1H), 7.31 (t, J=1.7 Hz, 1H), 7.12 (t, J=2.3 Hz, 1H), 6.78 (d, J=5.2 Hz, 1H), 3.85 (s, 3H), 2.77 (d, J=4.4 Hz, 3H)

### Example 67. 4-(3-Methoxy-5-(1H-pyrazol-1-yl) phenoxy) quinazoline (Compound 74)

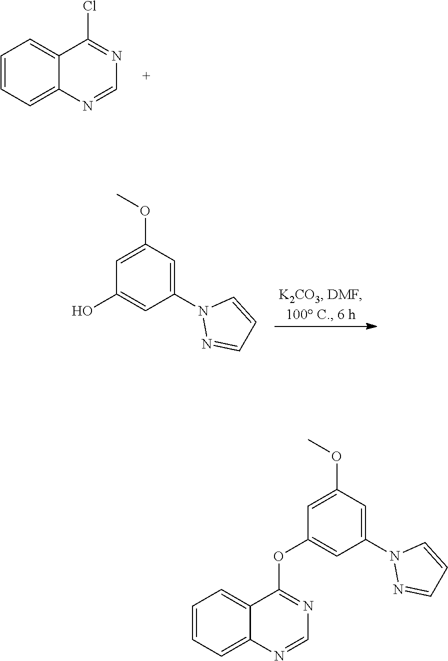 [SMILES: Clc1ncnc2ccccc12 ; COc1cc(O)cc(-n2cccn2)c1 ; "K2CO3, DMF,100° C., 6 h" ; COc1cc(Oc2ncnc3ccccc23)cc(-n2cccn2)c1]

4-3-methoxy-5-(1H-pyrazol-1-yl) phenol (70 mg, 0.36 mmol, 1.0 eq), 4-chloroquinazoline (73 mg, 0.44 mmol, 1.2 eq) and K₂CO₃ (102 mg, 0.73 mmol, 2 eq) were added to a round-bottom flask with a magnetic bar, then 2 ml DMF was added as a solvent. The reaction vessel was evacuated and backfilled with N₂ three times and protected with a balloon of N₂. The reaction mixture was heated at 100° C. for 6 h with vigorous stirring. The cooled solution was diluted with water (30 mL) and extracted with ethyl acetate (3×20 mL). Combined organic layers was washed with water (2×20 mL), brine (20 mL) and dried over anhydrous Na₂SO₄ and concentrated under reduced pressure. The residue was purified by silica gel flash chromatography to afford the product as a white solid. (40 mg, yield=34%, purity=96.1%)

TLC R_{f}=0.4 (EA/PE=6:4)

MS (ESI⁺): m/z=319.2 (M+1)

¹H NMR (400 MHz, CDCl₃): δ 8.81 (s, 1H), 8.38 (d, J=8.1 Hz, 1H), 8.04 (d, J=8.4 Hz, 1H), 7.94 (dd, J=10.6, 5.2 Hz, 2H), 7.74-7.66 (m, 2H), 7.27 (s, 1H), 7.24 (d, J=1.8 Hz, 1H), 6.77 (t, J=2.0 Hz, 1H), 6.47 (d, J=1.8 Hz, 1H), 3.90 (s, 3H).

### Example 68. 4-(3-methoxy-5-(1H-pyrazol-1-yl)phenoxy)-6,7-dimethoxyquinazoline (Compound 74)

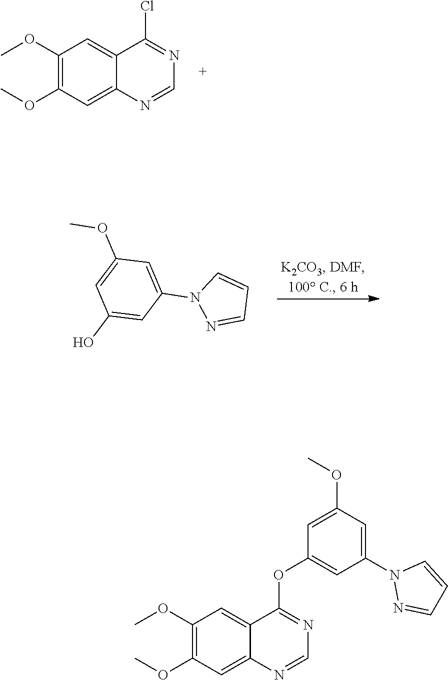 [SMILES: COc1cc2ncnc(Cl)c2cc1OC ; COc1cc(O)cc(-n2cccn2)c1 ; "K2CO3, DMF,100° C., 6 h" ; COc1cc(Oc2ncnc3cc(OC)c(OC)cc23)cc(-n2cccn2)c1]

3-methoxy-5-(1H-pyrazol-1-yl)phenol (70 mg, 0.36 mmol, 1.0 eq), 4-chloro-6,7-dimethoxyquinazoline (91 mg, 0.4 mmol, 1.1 eq) and K₂CO₃ (102 mg, 0.73 mmol, 2 eq) were added to a round-bottom flask with a magnetic bar, then 2 ml DMF was added as a solvent. The reaction vessel was evacuated and backfilled with N₂ three times and protected with a balloon of N₂. The reaction mixture was heated at 100° C. for 6 h with vigorous stirring. The cooled solution was diluted with water (30 mL) and extracted with ethyl acetate (3×20 mL). Combined organic layers was washed with water (2×20 mL), brine (20 mL) and dried over anhydrous Na₂SO₄ and concentrated under reduced pressure. The residue was purified by silica gel flash chromatography to afford the product as a white solid. (70 mg, yield=50%, purity=99%)

TLC R_{f}=0.5 (EA/PE=6:4)

MS (ESI): m/z=379.2 (M+1)

¹H NMR: (400 MHz, DMSO-d6) δ 8.58 (d, J=3.6 Hz, 2H), 7.75 (d, J=1.5 Hz, 1H), 7.58 (s, 1H), 7.44 (d, J=1.9 Hz, 1H), 7.43-7.39 (m, 2H), 6.90 (t, J=2.1 Hz, 1H), 6.56-6.53 (m, 1H), 3.99 (d, J=5.7 Hz, 6H), 3.86 (s, 3H).

### Example 69. 4-(3-Methoxy-5(4-methyl-H-imidazol-1-yl)phenoxy)-6,7-dimethoxyquinazoline (Compound 75)

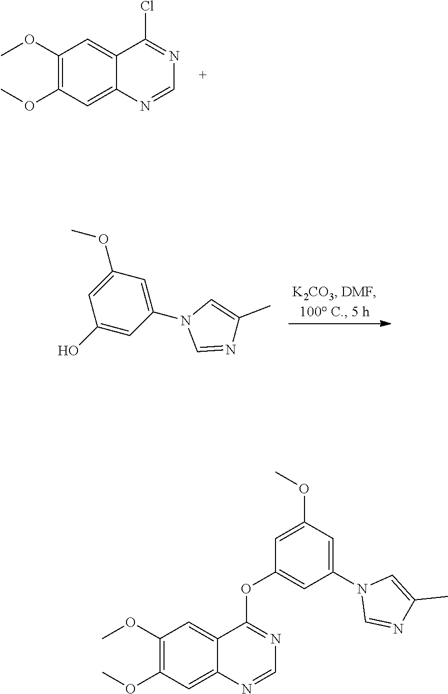

3-methoxy-5-(4-methyl-1H-imidazol-1-yl)phenol (70 mg, 0.34 mmol, 1.0 eq), 4-chloro-6,7-dimethoxyquinazoline (85 mg, 0.37 mmol, 1.1 eq) and K₂CO₃ (95 mg, 0.68 mmol, 2 eq) were added to a round-bottom flask with a magnetic bar, then 2 ml DMF was added as a solvent. The reaction vessel was evacuated and backfilled with N₂ three times and protected with a balloon of N₂. The reaction mixture was heated at 100° C. for 5 h with vigorous stirring. The cooled solution was diluted with water (30 mL) and extracted with ethyl acetate (3×20 mL). Combined organic layers was washed with water (2×20 mL), brine (20 mL) and dried over anhydrous Na₂SO₄ and concentrated under reduced pressure. The residue was purified by silica gel flash chromatography to afford the product as a white solid. (60 mg, yield=45%, purity=96.9%)

TLC R_{f}=0.3 (100% EA)

MS (ESI⁺): m/z=393.2 (M+1)

¹H NMR: (400 MHz, DMSO-d6) δ 8.58 (s, 1H), 8.23 (d, J=1.0 Hz, 1H), 7.56 (s, 1H), 7.53 (s, 1H), 7.41 (s, 1H), 7.27 (s, 1H), 7.18 (s, 1H), 6.90 (s, 1H), 3.99 (d, J=7.3 Hz, 6H), 3.85 (s, 3H), 2.14 (s, 3H)

### Example 70. 4-(3-Methoxy-5-(4-methyl-1H-imidazol-1-yl)phenoxy)quinazoline (Compound 76)

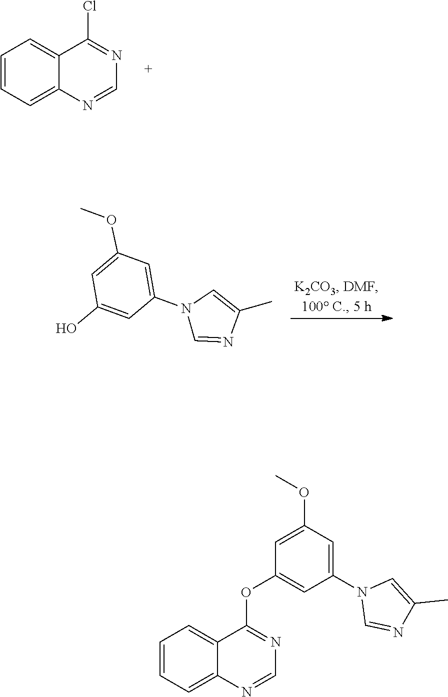 [SMILES: Clc1ncnc2ccccc12 ; COc1cc(O)cc(-n2cnc(C)c2)c1 ; "K2CO3, DMF,100° C., 5 h" ; COc1cc(Oc2ncnc3ccccc23)cc(-n2cnc(C)c2)c1]

4-methoxy-5-(4-methyl-1H-imidazol-1-yl)phenol (70 mg, 0.34 mmol, 1.0 eq), 4-chloroquinazoline (62 mg, 0.37 mmol, 1.1 eq) and K₂CO₃ (95 mg, 0.68 mmol, 2 eq) were added to a round-bottom flask with a magnetic bar, then 2 ml DMF was added as a solvent. The reaction vessel was evacuated and backfilled with N₂ three times and protected with a balloon of N₂. The reaction mixture was heated at 100° C. for 5 h with vigorous stirring. The cooled solution was diluted with water (30 mL) and extracted with ethyl acetate (3×20 mL). Combined organic layers was washed with water (2×20 mL), brine (20 mL) and dried over anhydrous Na₂SO₄ and concentrated under reduced pressure. The residue was purified by silica gel flash chromatography to afford the product as a white solid. (65 mg, yield=57%, purity=82%)

TLC R_{f}=0.25 (100% EA)

MS (ESI⁺): m/z=333.23 (M+1)

¹H NMR: (400 MHz, DMSO-d6) δ 8.77 (s, 1H), 8.39 (d, J=8.1 Hz, 1H), 8.23 (s, 1H), 8.11-8.00 (m, 2H), 7.82 (s, 1H), 7.53 (s, 1H), 7.31 (s, 1H), 7.20 (s, 1H), 6.96 (s, 1H), 3.85 (s, 3H), 2.14 (s, 3H).

### Example 71. 4-(3-Methoxy-5-(4-methyl-1H-imidazol-1-yl)phenoxy)quinoline (Compound 77)

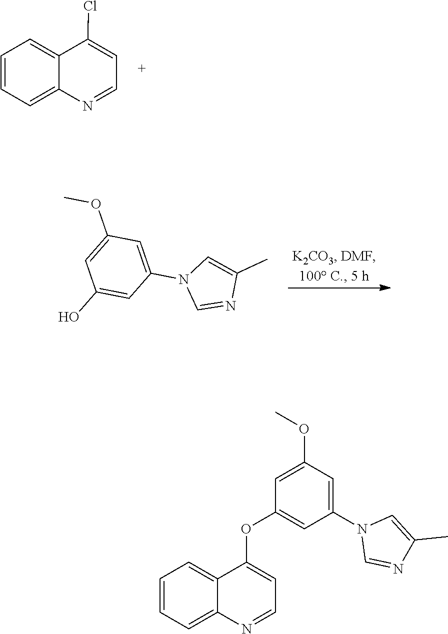

3-methoxy-5-(4-methyl-1H-imidazol-1-yl)phenol (100 mg, 0.48 mmol, 1.0 eq), 4-chloroquinoline (88 mg, 0.53 mmol, 1.1 eq) and K₂CO₃ (135 mg, 0.97 mmol, 2 eq) were added to a round-bottom flask with a magnetic bar, then 2 ml DMF was added as a solvent. The reaction vessel was evacuated and backfilled with N₂ three times and protected with a balloon of N₂. The reaction mixture was heated at 100° C. for 5 h with vigorous stirring. The cooled solution was diluted with water (30 mL) and extracted with ethyl acetate (3×20 mL). Combined organic layers was washed with water (2×20 mL), brine (20 mL) and dried over anhydrous Na₂SO₄ and concentrated under reduced pressure. The residue was purified by silica gel flash chromatography to afford the product as a white solid. (80 mg, yield=55.5%, purity=99%)

TLC R_{f}=0.15 (100% EA)

MS (ESI⁺): m/z=332.12 (M+1)

¹H NMR (400 MHz, DMSO) δ 8.73 (d, J=5.1 Hz, 1H), 8.31 (d, J=8.4 Hz, 1H), 8.26 (s, 1H), 8.06 (d, J=8.3 Hz, 1H), 7.84 (t, J=7.0 Hz, 1H), 7.68 (t, J=7.4 Hz, 1H), 7.56 (a, 1H), 7.26 (a, 1H), 7.21 (s, 1H), 6.86 (a, 1H), 6.79 (d, J=5.2 Hz, 1H), 3.85 (a, 3H), 2.14 (a, 3H).

### Example 72. 1H-imidazol-1-yl)-5-methoxyphenoxy)quinoline (Compound 78)

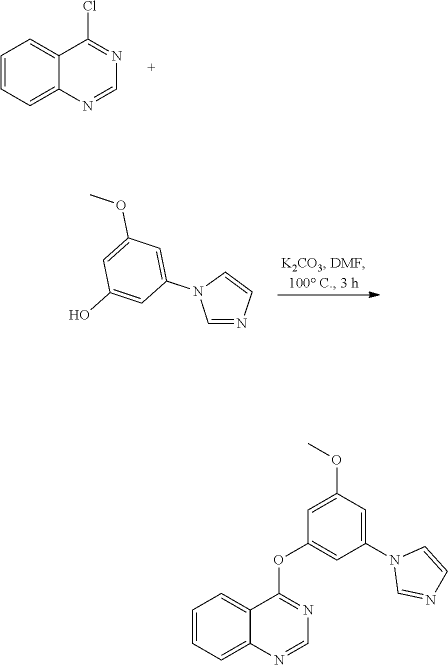 [SMILES: Clc1ncnc2ccccc12 ; COc1cc(O)cc(-n2ccnc2)c1 ; "K2CO3, DMF,100° C., 3 h" ; COc1cc(Oc2ncnc3ccccc23)cc(-n2ccnc2)c1]

3-(1H-imidazol-1-yl)-5-methoxyphenol (80 mg, 0.42 mmol, 1.0 eq), 4-chloroquinazoline (76 mg, 0.46 mmol, 1.1 eq) and K₂CO₃ (116 mg, 0.84 mmol, 2 eq) were added to a round-bottom flask with a magnetic bar, then 2 ml DMF was added as a solvent. The reaction vessel was evacuated and backfilled with N₂ three times and protected with a balloon of N₂. The reaction mixture was heated at 100° C. for 3 h with vigorous stirring. The cooled solution was diluted with water (30 mL) and extracted with ethyl acetate (3×20 mL). Combined organic layers was washed with water (2×20 mL), brine (20 mL) and dried over anhydrous Na₂SO₄ and concentrated under reduced pressure. The residue was purified by silica gel flash chromatography to afford the product as a white solid. (86 mg, yield=64%, purity=99%)

TLC R_{f}=0.2 (100% EA)

MS (ESI⁺): m/z=319.2 (M+1)

¹H NMR: (400 MHz, DMSO-d6) δ 8.77 (s, 1H), 8.42-8.34 (m, 2H), 8.06 (dt, J=14.3, 7.5 Hz, 2H), 7.87-7.80 (m, 2H), 7.38 (s, 1H), 7.27 (t, J=2.0 Hz, 1H), 7.10 (s, 1H), 7.00 (t, J=2.0 Hz, 1H), 3.86 (s, 3H)

### Example 73. 4-(3-(1H-Imidazol-1-yl)-5-methoxyphenoxy)-6,7-dimethoxyquinazoline (Compound 79)

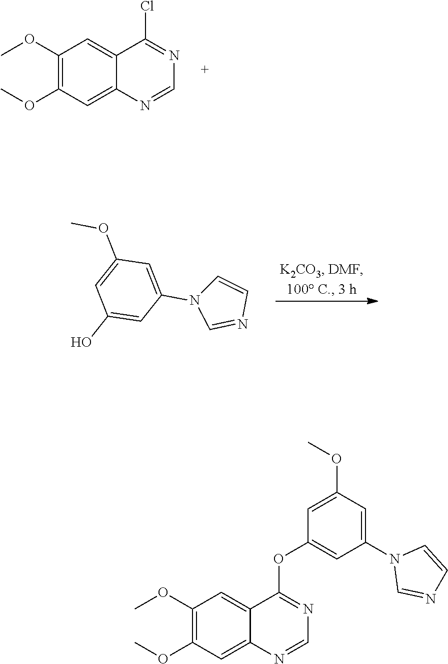 [SMILES: COc1cc2ncnc(Cl)c2cc1OC ; COc1cc(O)cc(-n2ccnc2)c1 ; "K2CO3, DMF,100° C., 3 h" ; COc1cc(Oc2ncnc3cc(OC)c(OC)cc23)cc(-n2ccnc2)c1]

3-(1H-imidazol-1-yl)-5-methoxyphenol (80 mg, 0.42 mmol, 1.0 eq), 4-chloro-6,7-dimethoxyquinazoline (104 mg, 0.46 mmol, 1.1 eq) and K₂CO₃ (116 mg, 0.84 mmol, 2 eq) were added to a round-bottom flask with a magnetic bar, then 2 ml DMF was added as a solvent. The reaction vessel was evacuated and backfilled with N₂ three times and protected with a balloon of N₂. The reaction mixture was heated at 100° C. for 3 h with vigorous stirring. The cooled solution was diluted with water (30 mL) and extracted with ethyl acetate (3×20 mL). Combined organic layers was washed with water (2×20 mL), brine (20 mL) and dried over anhydrous Na₂SO₄ and concentrated under reduced pressure. The residue was purified by silica gel flash chromatography to afford the product as a white solid. (89 mg, yield=56%, purity=99%)

TLC R_{f}=0.25 (100% EA)

MS (ESI⁺): m/z=379.2 (M+1)

¹H NMR: (400 MHz, DMSO-d6) δ 8.59 (s, 1H), 8.37 (s, 11H), 7.86 (s, 1H), 7.56 (a, 1H), 7.41 (s, 1H), 7.33 (s, 1H), 7.25 (s, 1H), 7.10 (s, 1H), 6.94 (s, 1H), 3.99 (d, J=6.9 Hz, 6H), 3.86 (a, 3H)

### Example 74. (S)-7-methoxy-4-(3-methoxy-5-((tetrahydrofuran-3-yl)oxy)phenoxy)quinoline-6-carboxamide (Compound 80)

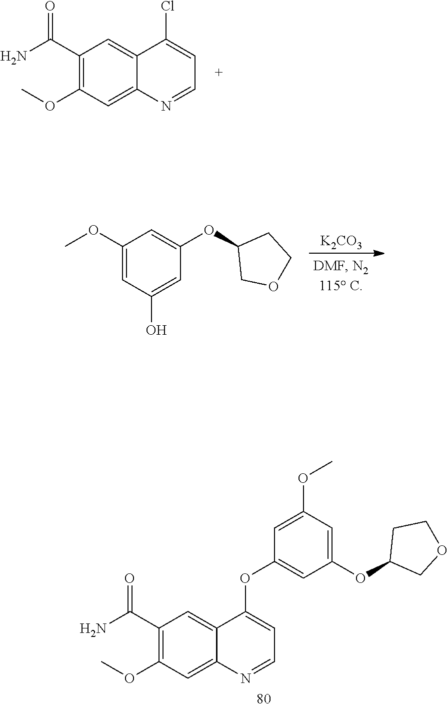

4-chloro-7-methoxyquinoline-6-carboxamide (119 mg, 1.0 mmol, 2.0 eq), (S)-3-methoxy-5-((tetrahydrofuran-3-yl)oxy)phenol (210 mg, 0.6 mmol, 1.2 eq) and K₂CO₃ (276 mg, 2.0 mmol, 4 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (190 mg, yield=92.7%, purity=98.2%)

TLC R_{f}=0.25 (DCM/MeOH=20/1)

MS (ESI⁺): m/z=411.10 (M+1)

¹H NMR: (400 MHz, DMSO-d6) δ 8.62 (d, J=5.3 Hz, 1H), 8.59 (s, 1H), 7.78 (s, 1H), 7.67 (s, 1H), 7.45 (a, 1H), 6.56 (d, J=5.3 Hz, 1H), 6.42 (t, J=2.1 Hz, 1H), 6.40 (p, J=2.2 Hz, 2H), 4.98 (ddt, J=6.2, 4.1, 1.8 Hz, 1H), 3.97 (s, 3H), 3.81-3.74 (m, 2H), 3.73 (t, J=1.6 Hz, 1H), 3.69 (s, 3H), 3.66 (dt, J=8.3, 4.2 Hz, 1H), 2.13 (dtd, J=16.5, 8.2, 6.2 Hz, 1H), 1.97-1.84 (m, 1H).

### Example 75. (R)-7-methoxy-4-(3-methoxy-5-((tetrahydrofuran-3-yl)oxy)phenoxy)quinoline-6-carboxamide (Compound 81)

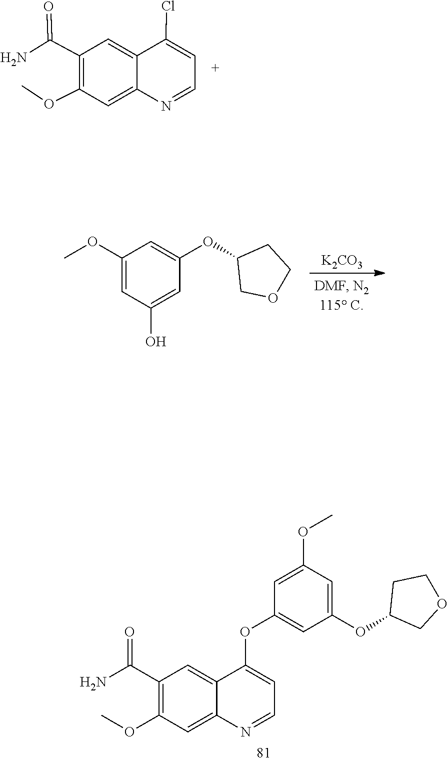

4-chloro-7-methoxyquinoline-6-carboxamide (119 mg, 0.5 mmol, 1.0 eq), (R)-3-methoxy-5-((tetrahydrofuran-3-yl)oxy)phenol (210 mg, 1.0 mmol, 2 eq) and K₂CO₃ (276 mg, 2.0 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (140 mg, yield=68.3%, purity=98.9%)

TLC R_{f}=0.4 (DCM/MeOH=20/1)

MS (ESI): m/z=411.8 (M+1)

¹H NMR: (400 MHz, DMSO-d6) δ 8.75 (d, J=5.3 Hz, 1H), 8.71 (s, 1H), 7.91 (s, 1H), 7.80 (s, 1H), 7.57 (s, 1H), 6.68 (d, J=5.3 Hz, 1H), 6.54 (t, J=2.1 Hz, 1H), 6.52 (p, J=2.2 Hz, 2H), 5.10 (ddt, J=6.2, 4.1, 1.8 Hz, 1H), 4.09 (s, 3H), 3.93-3.86 (m, 2H), 3.86-3.84 (m, 1H), 3.81 (s, 3H), 3.78 (dt, J=8.3, 4.2 Hz, 1H), 2.25 (dtd, J=13.4, 8.2, 6.2 Hz, 1H), 2.09-1.98 (m, 1H).

### Example 76. 4-(3,5-bis((tetrahydrofuran-3-yl)oxy)phenoxy)-7-methoxyquinoline-6-carbonamide (Compound 82)

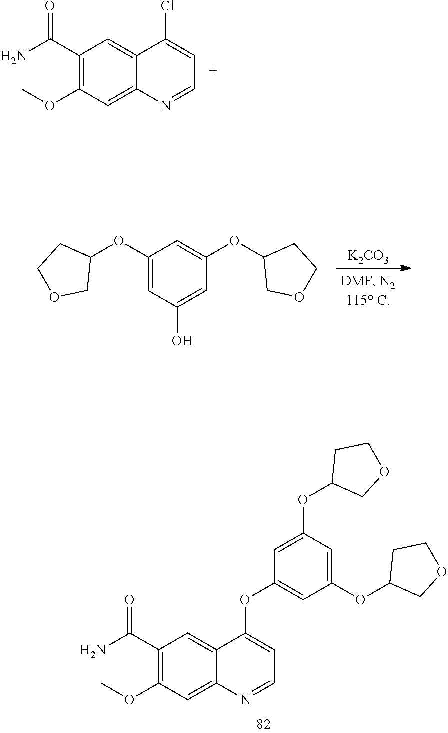

4-chloro-7-methoxyquinoline-6-carboxamide (119 mg, 0.5 mmol, 1.0 eq), 3,5-bis((tetrahydrofuran-3-yl)oxy)phenol (160 mg, 0.6 mmol, 1.2 eq) and K₂CO₃ (276 mg, 2.0 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (210 mg, yield=92.1%, purity=92.6%)

TLC R_{f}=0.3 (DCM/MeOH=20/1)

MS (ESI⁺): m/z=467.40 (M+1)

### Example 77. 7-methoxy-4-(3-methoxy-5-((tetrahydrofuran-3-yl)methoxy)phenoxy)quinoline-6-carboxamide (Compound 93)

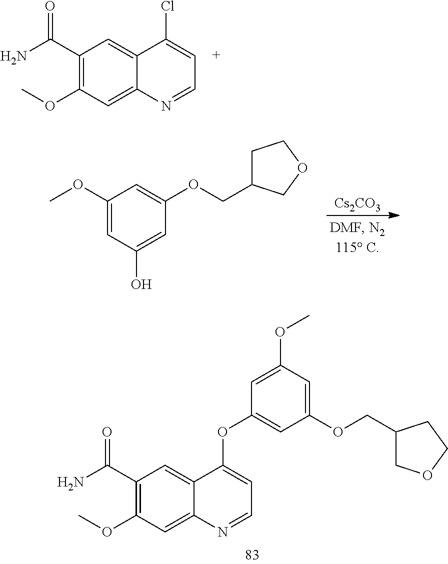

) 4-chloro-7-methoxyquinoline-6-carboxamide (119 mg, 0.5 mmol, 1.0 eq), 3-methoxy-5-((tetrahydrofuran-3-yl)methoxy)phenol (134 mg, 0.6 mmol, 1.2 eq) and K₂CO₃ (276 mg, 2.0 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (168 mg, yield=79.2%, purity=98.2%)

TLC R_{f}=0.4 (DCM/MeOH=20/1)

MS (ESI⁺): m/z=425.20 (M+1)

¹H NMR: (400 MHz, DMSO-d6) δ 8.74 (d, J=5.2 Hz, 1H), 8.71 (s, 1H), 7.96-7.87 (m, 1H), 7.79 (s, 1H), 7.57 (s, 1H), 6.67 (d, J=5.2 Hz, 1H), 6.54 (q, J=1.9 Hz, 3H), 4.09 (s, 3H), 4.04-3.89 (m, 2H), 3.82 (s, 3H), 3.81 (ddd, J=16.2, 8.3, 6.4 Hz, 2H), 3.69 (td, J=8.0, 6.8 Hz, 1H), 3.56 (dd, J=8.6, 5.5 Hz, 1H), 2.68 (p, J=6.5 Hz, 1H), 2.05 (dtd, J=12.7, 8.1, 5.6 Hz, 1H), 1.68 (ddt, J=12.6, 7.6, 6.2 Hz, 1H).

### Example 78. 7-methoxy-4-(3-methoxy-5-(3-methyl-H-pyrazol-1-yl)phenoxy)quinoline-6-carboxamide (Compound 84)

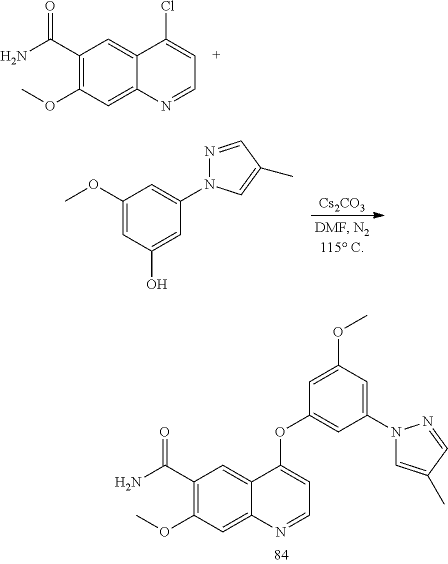

4-chloro-7-methoxyquinoline-6-carboxamide (119 mg, 0.5 mmol, 1.0 eq), 3-methoxy-5-(4-methyl-1H-pyrazol-1-yl)phenol (123 mg, 0.6 mmol, 1.2 eq) and K₂CO₃ (276 mg, 2.0 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (150 mg, yield=74.3%, purity=94.2%)

TLC R_{f}=0.2 (DCM/MeOH=20/1)

MS (ESI): m/z=405.10 (M+1)

### Example 79. 7-methoxy-4-(3-methoxy-5-(3-methyl-1H-pyrazol-1-yl)phenoxy)quinoline-6-carboxamide (Compound 85)

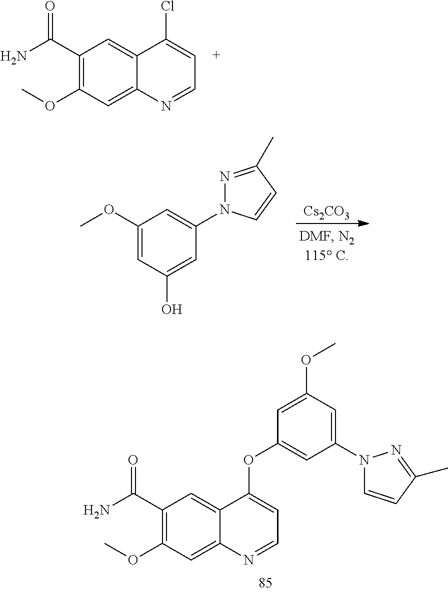

4-chloro-7-methoxyquinoline-6-carboxamide (142 mg, 0.6 mmol, 1.2 eq), 3-methoxy-5-(3-methyl-1H-pyrazol-1-yl)phenol (204 mg, 0.5 mmol, 1.0 eq) and K₂CO₃ (276 mg, 2.0 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (195 mg, yield=96.4%, purity=94.5%)

TLC R_{f}=0.25 (DCM/MeOH=40/1)

MS (ESI⁺): m/z=405.00 (M+1)

¹H NMR: (400 MHz, DMSO-d6) δ 8.73-8.69 (m, 2H), 8.47 (d, J=2.5 Hz, 1H), 7.89-7.85 (m, 1H), 7.76 (s, 1H), 7.55 (s, 1H), 7.38 (t, J=2.1 Hz, 1H), 7.33 (t, J=2.0 Hz, 1H), 6.82 (t, J=2.2 Hz, 1H), 6.69 (d, J=5.2 Hz, 1H), 6.34 (d, J=2.5 Hz, 1H), 4.05 (a, 3H), 3.86 (a, 3H), 2.25 (s, 3H).

### Example 80. 4-(3-(3,5-dimethyl-1H-pyrazol-1-yl)-5-methoxyphenoxy)-7-methoxyquinoline-6-carboxamide (Compound 86)

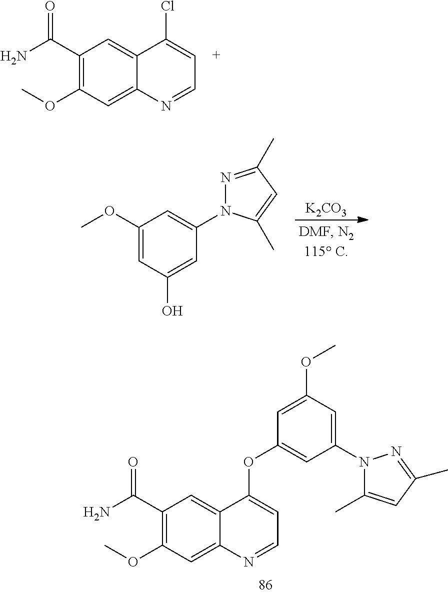

4-chloro-7-methoxyquinoline-6-carboxamide (118.4 mg, 0.5 mmol, 1.0 eq), 3-(3,5-dimethyl-1H-pyrazol-1-yl)-5-methoxyphenol (131 mg, 0.6 mmol, 1.2 eq) and K₂CO₃ (276 mg, 2.0 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the produce. (227 mg (containing some solvent), purity=96.23%)

TLC R_{f}=0.25 (DCM/MeOH=40/1)

MS (ESI⁺): m/z=419.20 (M+1)

### Example 81. 7-methoxy-4-(3-methoxy-5-(5-methyl-1,3,4-oxadiazol-2-yl)phenoxy)quinoline-6-carboxamide (Compound 7)

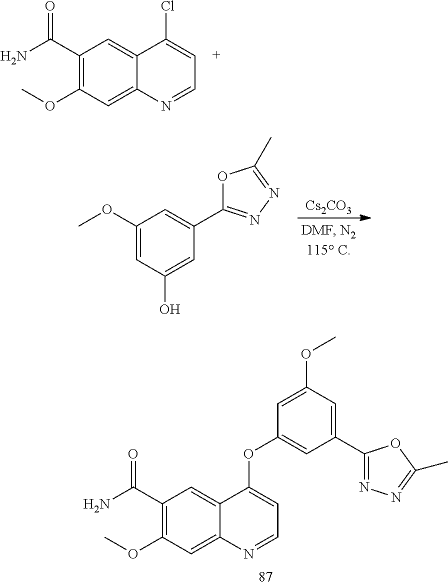

4-chloro-7-methoxyquinoline-6-carboxamide (162.8 mg, 0.69 mmol, 1.0 eq), 3-methoxy-5-(5-methyl-1,3,4-oxadiazol-2-yl)phenol (142.1 mg, 0.69 mmol, 1.0 eq) and K₂CO₃ (381 mg, 2.77 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (143 mg, yield=50.9%, purity=99.6%)

TLC R_{f}=0.3 (DCM/MeOH=20/1)

MS (ESI⁺): m/z=407.20 (M+1)

### Example 82. 6-bromo-7-methoxy-4-(3-methoxy-5-(1H-pyrazol-1-yl)phenoxy)quinoline (Compound 88)

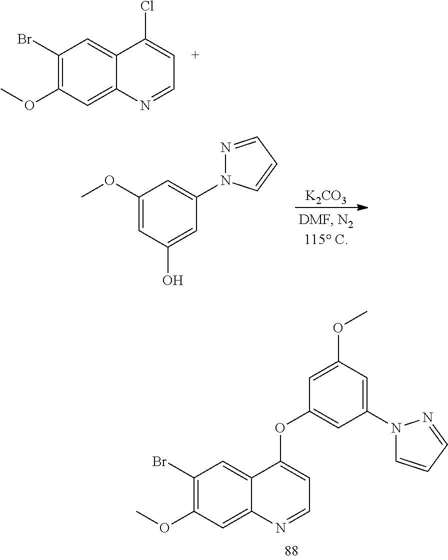

6-bromo-4-chloro-7-methoxyquinoline (163 mg, 0.6 mmol, 1.2 eq), 3-methoxy-5-(1H-pyrazol-1-yl)phenol (95 mg, 0.5 mmol, 1.0 eq) and K₂CO₃ (276 mg, 2.0 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (172 mg, yield=80.9%, purity=93.5%)

TLC R_{f}=0.4 (PE/EA=1/2)

MS (ESI⁺): m/z=426.10 (M+1)

### Example 83. 6-bromo-4-(3,5-dimethoxyphenoxy)-7-methoxyquinone (Compound 89)

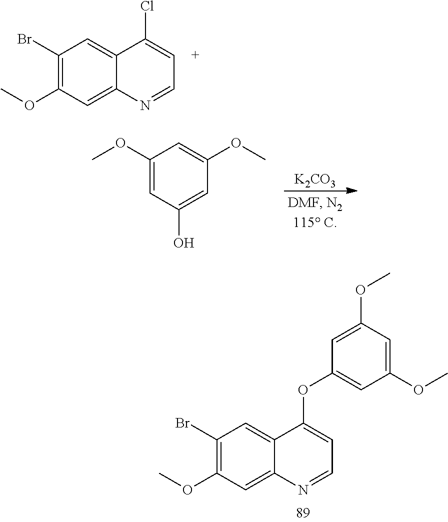

6-bromo-4-chloro-7-methoxyquinoline (136 mg, 0.5 mmol, 1.0 eq), 3,5-dimethoxyphenol (92.4 mg, 0.6 mmol, 1.2 eq) and K₂CO₃ (276 mg, 2.0 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (134 mg, yield=68.9%, purity=99%)

TLC R_{f}=0.2 (PE/EA=1/1)

MS (ESI⁺): m/z=390.00 (M+1)

### Example 84. 3-((6-bromo-7-methoxyquinolin-4-yl)oxy)-5-methoxy-N-methylbenzamide (Compound 90)

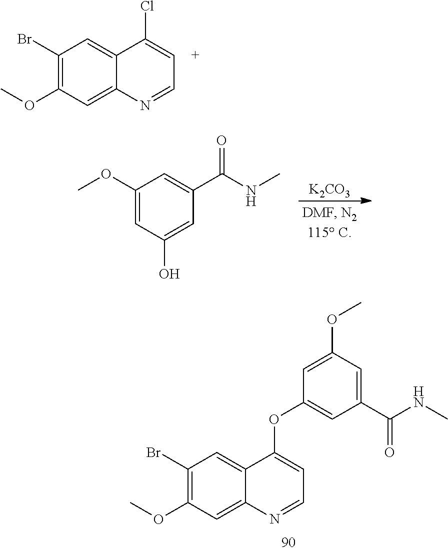

6-bromo-4-chloro-7-methoxyquinoline (163.5 mg, 0.6 mmol, 1.2 eq), 3-hydroxy-5-methoxy-N-methylbenzamide (91 mg, 0.5 mmol, 1.0 eq) and K₂CO₃ (276 mg, 2.0 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as a white solid. (117 mg, yield=56.1%, purity=99%)

TLC R_{f}=0.2 (PE/EA=1/4)

MS (ESI): m/z=417.00 (M+1)

### Example 85. 3-((6-bromoquinolin-4-yl)oxy)-5-methoxyaniline (Compound 91)

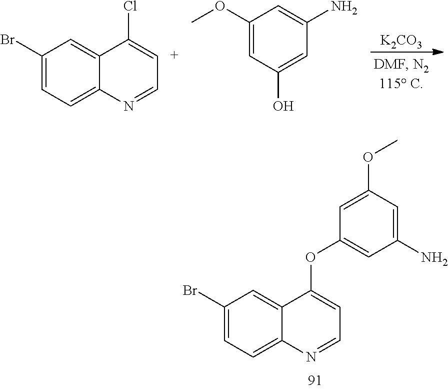

6-bromo-4-chloro-7-methoxyquinoline (1273 mg, 5.28 mmol, 1.2 eq), 3-amino-5-methoxyphenol (611 mg, 4.4 mmol, 1.0 eq) and K₂C₀₃ (2430 mg, 17.6 mmol, 4.0 eq) were added to a round-bottom flask with a magnetic bar, then 30 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 200 ml ethyl acetate and washed with brine. The organic phase was dried over anhydrous Na₂SO₄ and concentrated in vacuum. The residue was purified by silica gel flash chromatography to afford the product as a white solid. (613 mg, yield=40.5%, purity=96.1%)

TLC R_{f}=0.15 (PE/EA=2/1)

MS (ESI⁺): m/z=345.10 (M+1)

### Example 6. 6-bromo-4-(3-methoxy-5-((tetrahydrofuran-3-yl)oxy)phenoxy)quinoline (Compound 92)

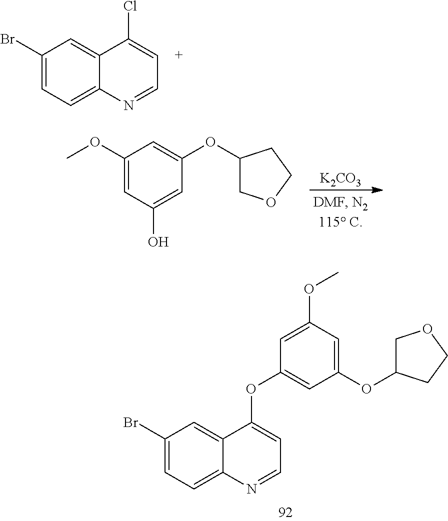

6-bromo-4-chloro-7-methoxyquinoline (195 mg, 0.8 mmol, 1.0 eq), 3-methoxy-5-((tetrahydrofuran-3-yl)oxy)phenol (168 mg, 0.8 mmol, 1.0 eq) and K₂CO₃ (442 mg, 3.2 mmol, 4.0 eq) were added to a round-bottom flask with a magnetic bar, then 5 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 30 ml ethyl acetate and washed with brine. The organic phase was dried over anhydrous Na₂SO₄ and concentrated in vacuum. The residue was purified by silica gel flash chromatography to afford the product. (308 mg, yield=92.6%, purity=99%)

TLC R_{f}=0.45 (PE/EA=2/1)

MS (ESI⁺): m/z=417.80 (M+1)

### Example 87. 4-(3-methoxy-5-nitrophenoxy)-6-bromoquinone (Compound 93)

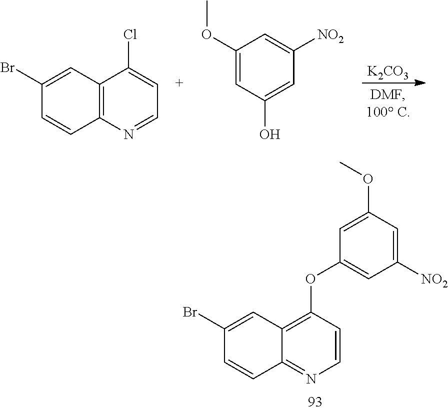

6-bromo-4-chloroquinoline (287 mg, 1.18 mmol, 1 eq), 3-methoxy-5-nitrophenol (200 mg, 1.18 mmol, 1 eq) and K₂CO₃ (327 mg, 2.4 mmol, 2 eq) were added to a round-bottom flask with a magnetic bar, then 4 mL DMF was added as solvent. The reaction vessel was evacuated and backfilled with N₂ three times and protected with a balloon of N₂. The reaction mixture was heated at 130° C. for at least 12 h with vigorous stirring. The cooled solution was diluted with 20 mL ethyl acetate and washed with brine. The organic phase was dried over anhydrous Na₂SO₄, and concentrated in vacuo. The residue was purified by silica gel flash chromatography to afford the product as a white solid. (250 mg, yield=45%).

TLC R_{f}=0.2 (PE EA=7/3)

MS (ESI⁺): m/z==375 (M+H)

¹H NMR (400 MHz, DMSO-d) 88.78 (d, J=5.1 Hz, 1H), 8.45 (d, J=1.1 Hz, 1H), 8.02 (d, J=8.8 Hz, 1H), 7.98 (dd, J=9.0, 1.8 Hz, 1H), 7.76 (a, 1H), 7.72 (d, J=1.7 Hz, 1H), 7.48 (s, 1H), 6.93-6.87 (m, 1H), 3.91 (a, 3H).

### Example 8. 3-((6-bromoquinone-4-yl)oxy)-N-methyl-5-(trifluromethyl)benzamide (Compound 94)

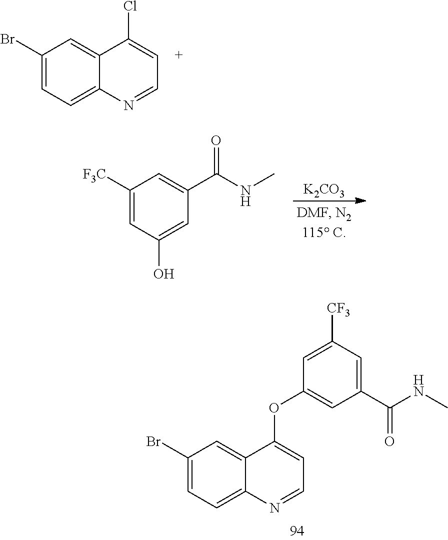

6-bromo-4-chloro-7-methoxyquinoline (145.2 mg, 0.6 mmol, 1.2 eq), 3-hydroxy-N-methyl-5-(trifluoromethyl)benzamide (109.5 mg, 0.5 mmol, 1.0 eq) and K₂CO₃ (276 mg, 2.0 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product. (195 mg, yield=92%, purity=99.2%)

TLC R_{f}=0.25 (PE/EA=1/1)

MS (ESI⁺): m/z=427.00 (M+1)

### Example 89. 4-(3-acetamido-5-methoxyphenoxy)-6-bromoquinoline 1-oxide (Compound 95)

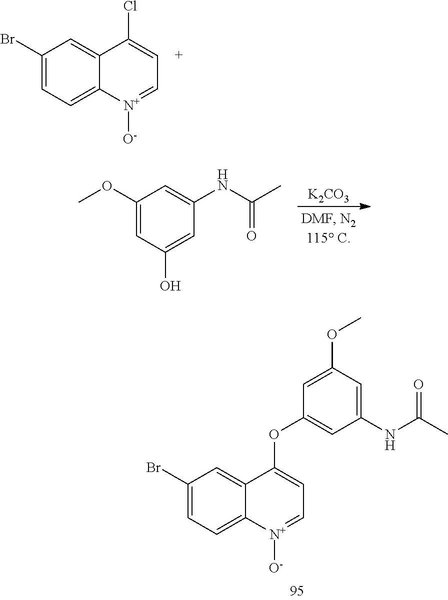

6-bromo-4-chloroquinoline 1-oxide (154 mg, 0.5 mmol, 1.0 eq), N-(3-hydroxy-5-methoxyphenyl)acetamide (91 mg, 0.5 mmol, 1.0 eq) and K₂CO₃ (276 mg, 2.0 mmol, 4.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product. (144 mg, yield=70.6%, purity=96.7%)

TLC R_{f}=0.5 (DCM/MeOH=20/1)

MS (ESI⁺): m/z=403.20 (M+1)

### Example 90. 4-(3-methoxy-5-(1H-pyrazol-1-yl)phenoxy)-2-methylquinoline (Compound 96)

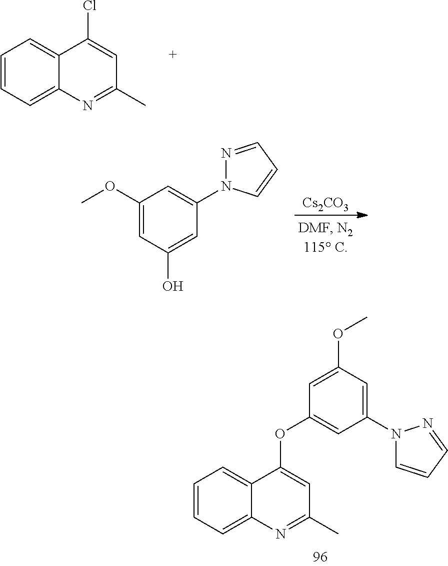

6-bromo-4-chloro-2-methylquinoline (106.6 mg, 0.6 mmol, 1.2 eq), 3-methoxy-5-(1H-pyrazol-1-yl)phenol (95 mg, 0.5 mmol, 1.0 eq) and Cs₂CO₃ (325.8 mg, 1.0 mmol, 2.0 eq) were added to a round-bottom flask with a magnetic bar, then 3 ml DMF was added as solvent. The reaction vessel was evacuated and backfilled with N₂ three times and protected with a balloon of N₂. The reaction mixture was heated at 115° C. for at least 12 h with vigorous stirring. The cooled solution was diluted with 20 ml ethyl acetate and washed with brine. The organic phase was dried over anhydrous Na₂SO₄ and concentrated in vacuum. The residue was purified by silica gel flash chromatography to afford the product as an oil. (142 mg, yield=85.7%, purity=98.98%)

TLC R_{f}=0.33 (PE/EA=2/1)

MS (ESI⁺): m/z=332.20 (M+1)

### Example 91. 4-(3-methoxy-5-nitrophenoxy)-6-bromo-2-chloroquinoline (Compound 97)

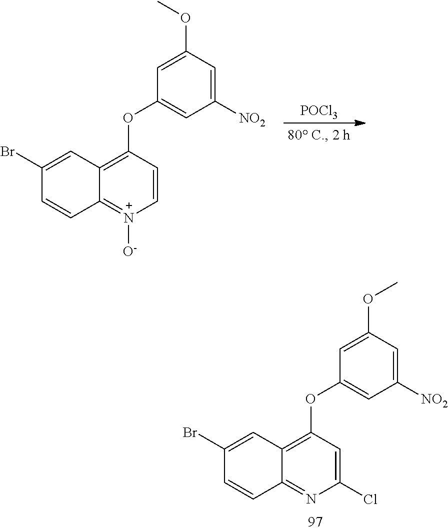 [SMILES: COc1cc(Oc2cc[n+]([O-])c3ccc(Br)cc23)cc([N+](=O)[O-])c1 ; "POCl3" ; COc1cc(Oc2cc(Cl)nc3ccc(Br)cc23)cc([N+](=O)[O-])c1]

6-bromo-4-(3-methoxy-5-nitrophenoxy)-1-quinolin-1-olate (600 mg, 1.53 mmol, 1 eq) in POCl₃ (6 mL) was heated at 80° C. for 2 h. Solvent was evaporated under reduced pressure, crude was diluted with sat. Na₂CO₃ solution and extracted with EA (40 mL). Organic layer was washed with water, brine solution, dried over Na₂SO₄ filtered and evaporated under reduced pressure. Crude material was purified by trituration using pentane to afford the product as a pale yellow solid. (500 mg, yield=80%).

MS (ESI⁺): m/z=411 (M+H)

¹H NMR (400 MHz, DMSO-d₆) δ 8.45 (d, J=2.2 Hz, 1H), 8.05 (dd, J=9.0, 2.2 Hz, 1H), 7.94 (d, J=9.0 Hz, 1H), 7.85 (t, J=2.0 Hz, 1H), 7.75 (t, J=2.2 Hz, 1H), 7.53 (t, J=2.2 Hz, 1H), 6.88 (s, 1H), 3.92 (s, 3H).

### Example 92. Cell Culture and Treatment

Six lung cancer cell lines (NCI-H23, NCI-H460, NCI-H596, NCI-H2170, Calu-6 and A549), two colon cancer cell lines (HCT116 and SW460), one breast cancer cell line MDA-MB-231, one gastric cancer cell line NCI-N87, one prostate cancer cell line DU145, one cervical cancer cell line Hela, and one glioblastoma cell line T98G were obtained from ATCC. The T2 HCC cell line was derived from a mouse liver cancer model initiated by a transgene of MYC. All cell lines were cultured in DMEM (Gibco, Cleveland, TN, USA) supplemented with 5% fetal bovine serum (Gibco), penicillin (100 U/mL)—streptomycin (100 μg/mL) (Gibco, Cat. No. 15140-122), 2 mM L-glutamine (Gibco, 200 mM solution, Cat. No. 25030081), and 1 mM sodium pyruvate (Gibco, 100 mM solution, Cat. No. 11360070) at 37° C. in a humidified incubator that was maintained at 5% CO2.

### Example 93. Phenotypic Screening Assays

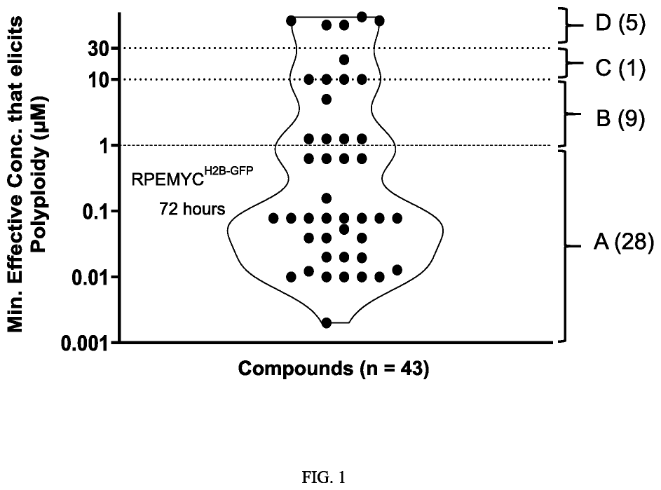

We have previously developed a mechanism-informed phenotypic screening assay to identify novel antimitotic agents. Equipped with a prior understanding of the versatile functions of the chromosomal passenger protein (CPP) complex in orchestrating karyokinesis and cytokinesis, the screening assay is to score for phenotypes typically seen when the CPP complex is disabled. Specifically, the parameters for a positive hit are a temporary elevation of mitotic index (MI) at 24 hours of drug treatment and an accumulation of polyploid cells at 48 hours of drug treatment, indicative of mitotic arrest and cytokinetic failure respectively. These parameters exclude compounds that elicit only a prolonged arrest of cells in mitosis, a phenotype typically provoked by spindle toxins. The screening procedure is briefly summarized here. RPEMYC^{H2B-GFP} cells engineered to express a Histone 2B-EGFP fusion protein were passaged as batches of 96-well plates, 18-24 hours before exposure to the chemical compounds of the present disclosure at concentrations from 1 nM to 30 μM. At 24, 48 or 72 hours after initiation of treatment, cells were analyzed for either an arrest in mitosis or a change in DNA content by GE IN-Cell Analyzer 2000. Testing results of 43 compounds were summarized in FIG. 1 and Table 2. As set forth in Table 2 below, a value of greater than or equal to 1 nM and less than or equal to 1.0 μM is marked “A”; a value greater than 1.00 μM and less than or equal to 10.0 μM is marked “B”; a value greater than 10.0 μM and less than or equal to 30.0 μM is marked “C”; and a value greater than 30.0 μM is marked “D.”

TABLE 2

| | Mitotic | Min. Effective | | |
|---|---|---|---|---|
| | Arrest | conc. for | Along with | Proliferative |
| | (≥5%) | Polyploidy (≥5%) | Cell Death | arrest (>50%) |
| NO. | (μM) | (μM) | (μM) | (μM) |
| 1 | A | A | A | |
| 2 | D | D | D | |
| 3 | A | A | A | |
| 4 | A | A | A | |
| 5 | B | B | B | |
| 6 | A | A | A | |
| 7 | A | A | A | A |
| 8 | A | A | A | A |
| 9 | D | D | D | D |
| 10 | A | B | B | B |
| 11 | B | B | B | B |
| 12 | A | A | A | A |
| 13 | A | A | A | A |
| 14 | A | A | A | A |
| 15 | A | A | A | A |
| 16 | B | B | B | B |
| 17 | A | A | A | A |
| 18 | A | A | A | A |
| 19 | A | A | A | A |
| 20 | A | A | A | A |
| 21 | A | A | A | A |
| 22 | A | A | A | A |
| 23 | B | B | B | B |
| 24 | C | C | C | C |
| 25 | D | D | D | D |
| 26 | A | A | A | A |
| 27 | A | A | A | A |
| 28 | A | B | B | B |
| 29 | A | A | A | A |
| 30 | A | A | A | A |
| 31 | A | A | A | A |
| 32 | A | A | A | A |
| 33 | B | B | B | B |
| 34 | A | A | A | A |
| 35 | A | A | A | A |
| 36 | A | A | A | A |
| 37 | A | A | A | A |
| 38 | A | A | A | A |
| 39 | A | A | A | A |
| 40 | A | A | A | A |
| 41 | A | A | A | A |
| 42 | A | A | A | A |
| 43 | A | A | A | A |
| 44 | B | B | B | B |
| 45 | B | B | B | B |
| 46 | A | A | A | A |
| 47 | B | B | B | B |
| 48 | A | A | A | A |
| 49 | A | A | A | A |
| 50 | A | A | A | A |
| 51 | A | A | A | A |
| 52 | B | C | C | C |
| 53 | A | A | A | A |
| 54 | B | C | C | C |
| 55 | A | A | A | A |
| 56 | B | C | C | C |
| 57 | A | A | A | A |
| 58 | B | B | B | B |
| 59 | A | A | A | A |
| 60 | A | A | A | A |
| 61 | A | A | A | A |
| 62 | A | A | A | A |
| 63 | A | A | A | A |
| 64 | A | A | A | A |
| 65 | A | A | A | A |
| 66 | A | A | A | A |
| 67 | A | A | A | A |
| 68 | A | A | A | A |
| 69 | A | A | A | A |
| 70 | B | B | B | B |
| 71 | B | B | B | B |
| 72 | A | A | A | A |
| 73 | C | C | C | C |
| 74 | C | C | C | C |
| 75 | A | A | A | A |
| 76 | B | B | B | B |
| 77 | A | A | A | A |
| 78 | B | B | B | B |
| 79 | C | C | C | C |
| 80 | A | A | A | |
| 81 | A | A | A | |
| 82 | B | B | B | |
| 83 | A | A | A | |
| 84 | A | A | A | |
| 85 | A | A | A | |
| 86 | B | B | B | |
| 87 | A | A | A | |
| 88 | A | A | A | |
| 89 | A | A | A | |
| 90 | B | B | B | |
| 91 | A | A | A | |
| 92 | A | A | A | |
| 93 | A | A | A | |
| 94 | B | B | B | |
| 95 | A | A | A | |
| 96 | A | A | A | |
| 97 | D | D | D | |
| 98 | B | B | B | |

### Example 94. Colony Formation Assay of the Long-Term Effect of Anticancer Agents

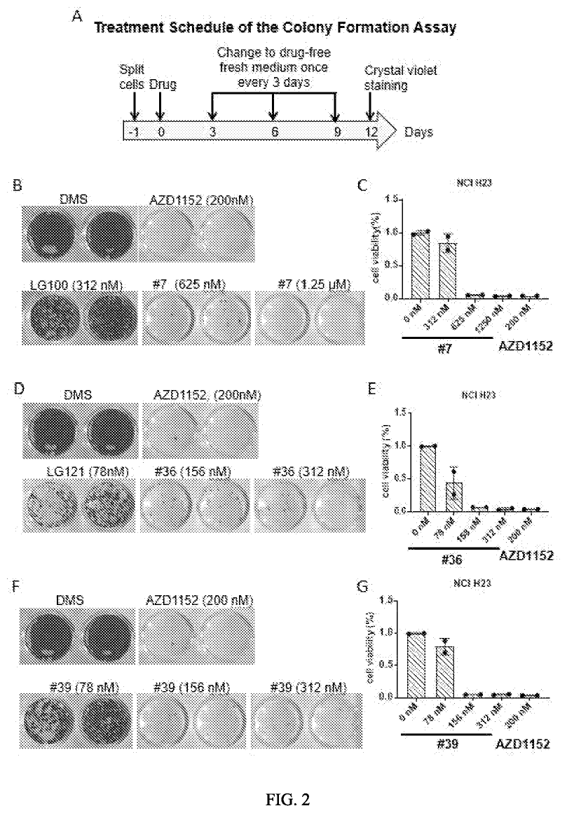

Acute cytotoxicity immediately determined after short-term exposure to antimitotic agents often underestimates their potency, because some cancer cells might not die quickly after suffering mitotic defects such as an arrest in mitosis or a cytokinetic failure. Instead, after exposed to antimitotic agents, cells might face multiple possibilities that adversely affect their viability and proliferation in long-term such as permanent arrest of proliferation due to the development of senescence and nonapoptotic cell death by excessive autophagy. Only a small fraction of cells pretreated with an antimitotic agent might resume proliferation and divide to generate viable daughter cells that form colonies. Assay of the ability of cells to form colonies after being exposed to an anticancer agent for a short period of time represents a more accurate approach to document the potency of antimitotic agents. The human lung cancer cell line NCI-H23 in sub-confluence was exposed to compounds #7, #36, and #39 for three days and then transferred to drug-free fresh medium once every 3 days until 12 days after the initiation of treatment. At the end point, cells were photographed after fixed and stained with crystal violet. All three compounds inhibited the long-term proliferative potential of NCI-H23 cells as potent as AZD1152, an inhibitor of the mitotic kinase Aurora B that reached clinical trials (FIG. 2).

### Example 95. Soft Apr Colony Formation Assay

Measuring the ability of cells to grow in soft agar has been popularly believed as the gold standard assay for cellular transformation in vitro. In the Soft Agar Assay, cells grow from single cells to cell colonies in a semi-solid agar solution that keeps them away from the solid surface and allows growth in an anchorage-independent way.

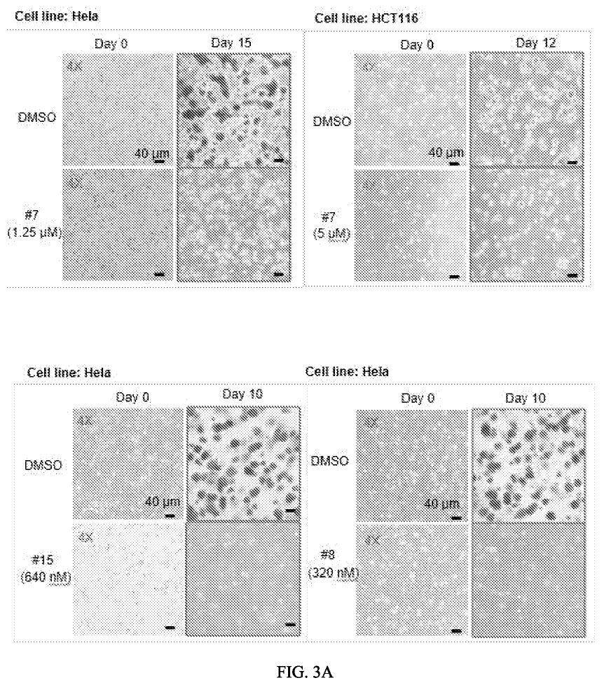

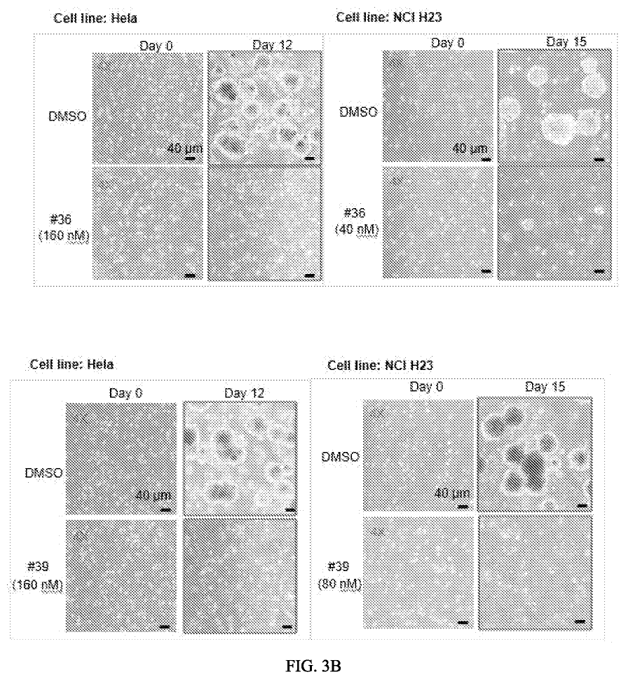

The anchorage-independent growth of cells is one of the hallmarks of cancer cells. Normal epithelial cells are supported by basement membranes that provide survival and proliferative signals while undergo a type of apoptosis called anoikis when lose their attachment to the extracellular matrix. Cancer cells, in contrast, evade detachment-induced apoptosis, leading to uncontrolled proliferation and metastasis. The Soft Agar Colony Formation Assay allows testing of the therapeutic efficacy of compounds against anchorage-independent 3D growth of cancer cells in vin. The assay was performed in 6-well plates with two layers of agar. For the first, 0.75% agar in DMEM medium was melted in a microwave oven and poured to form a bottom layer. Once solidified, 10-100K cells in 1 ml of DMEM containing 0.35% agar was added to form the top layer, which was later covered with 0.5 ml of DMEM. Cell culture medium was changed once every two days until colonies were ready to photograph. We test the antitumor activity of select compounds in soft agar colony formation assays. Data are summarized in FIG. 3. Compounds #7, #8, #15, #36, and #39 effectively suppressed the anchorage-independent growth of a cervical cancer cell line Hela. Similarly, all compounds tested, including #36 and #39, completely blocked the growth of a human lung cancer cell line NCI-H23 in soft agar. Compound #7 also suppressed the growth a colon cancer cell line HCT116 in soft agar. The suppression of growth of these three cancer cell lines in 3D culture is consistent with the potent impact of these compounds on cellular proliferation in 2D culture.

### Example 96. The MTT Assay of Cellular Proliferation and Determination of EC₅₀

The MTT assay measures cellular metabolic activity as a proxy for cell viability and involves the conversion of the water-soluble yellow dye MTT [3-(4,5-dimethylthiazol-2-yl)-2,5-diphenyltetrazolium bromide] into an insoluble purple formazan by the action of mitochondrial reductase. Formazan is then solubilized and its concentration is determined by measuring the optical density (OD) value at a wavelength of 570 nm. The value is in proportional to the number of live cells with excellent linearity up to ˜10⁶ cells per well. The MTT assay was used to determine the EC₅₀ value, the concentration of a compound that leads to 50% inhibition of cellular proliferation. Briefly, cells were split when growing to the mid-Log phase. Cells in 100 μL of culture medium were seeded into each well of 96-well microplates and cultivated for 15-24 hours to reach a confluence of 20-30% and were then exposed to drugs at concentrations ranging from 1 nM to 30 μM. At the endpoint, 20 μL of a MTT stock solution in DMSO (5 mg/mL) was added to each well that contains 100 μL of DMEM. The microplates were left in the cell culture incubator for 3-4 h before subjected to solubilization and determination of formazan at A570 in a microplate reader (BioTek ELX808iu). The results of these assays are summarized in Table 3. As set forth in table 2 below, a value of greater than or equal to 1 nM and less than or equal to 1.0 μM is marked “A”; a value greater than 1.00 M and less than or equal to 10.0 μM is marked “B”; a value greater than 10.0 μM and less than or equal to 30.0 μM is marked “C”; and a value greater than 30.0 μM is marked “D.” The LG series of compounds displayed potent activity in all human cancer cell lines tested, including six lung cancer cell lines (NCI-H23, NCI-H460, NCI-H596, NCI-H2170, Calu-6 and A549), two colon cancer cell lines (HCT116 and SW460), one breast cancer cell line MDA-MB-231, one gastric cancer cell line NCI-N87, one prostate cancer cell line DU145, one cervical cancer cell line Hela, one glioblastoma cell line T98G, and one liver cancer cell line T2 HCC. Therefore, these compounds might hold a broad utility in the treatment of a large variety of human malignancies.

TABLE 3

| | | NCI- | NCI- | NCI- | NCI- | | | | | MDA- | | NCI- | | | T2 |
|---|---|---|---|---|---|---|---|---|---|---|---|---|---|---|---|
| No | | H23 | H460 | H596 | H2170 | A549 | HCT116 | SW480 | T98G | MB-231 | Hela | N87 | DU145 | Calu-6 | HCC |
| 7 | MECP | A | B | A | — | — | A | — | A | A | A | — | — | — | B |
| | EC50 | A | B | B | — | — | A | — | A | A | B | — | — | — | B |
| 8 | MECP | A | B | A | — | — | A | — | B | A | A | — | — | — | A |
| | EC50 | A | D | B | — | — | A | — | B | A | A | — | — | — | A |
| 12 | MECP | A | — | — | — | A | A | A | — | A | A | A | — | — | A |
| | EC50 | A | — | — | — | A | A | A | — | A | A | A | — | — | A |
| 14 | MECP | A | — | — | — | A | A | A | — | A | A | A | — | — | A |
| | EC50 | A | — | — | — | A | A | A | — | A | A | A | — | — | A |
| 15 | MECP | A | B | A | — | — | A | — | B | A | A | — | — | — | A |
| | EC50 | A | D | B | — | — | A | — | B | A | A | — | — | — | A |
| 17 | MECP | A | — | — | — | A | A | A | — | A | A | A | — | — | A |
| | EC50 | A | — | — | — | A | A | A | — | A | A | A | — | — | A |
| 18 | MECP | A | — | — | — | A | A | A | — | A | A | A | — | — | A |
| | EC50 | A | — | — | — | A | A | A | — | A | A | A | — | — | A |
| 19 | MECP | A | — | — | — | A | A | A | — | A | A | A | — | — | A |
| | EC50 | A | — | — | — | A | A | B | — | A | A | A | — | — | A |
| 20 | MECP | A | — | — | — | A | A | A | — | A | A | A | — | — | A |
| | EC50 | A | — | — | — | A | A | A | — | A | A | A | — | — | A |
| 21 | MECP | A | — | — | — | A | A | A | — | A | A | A | — | — | A |
| | EC50 | A | — | — | — | A | A | A | — | A | A | A | — | — | A |
| 22 | MECP | A | — | — | — | A | A | A | — | A | A | A | — | — | A |
| | EC50 | A | — | — | — | A | A | A | — | A | A | A | — | — | A |
| 26 | MECP | A | — | — | — | A | A | A | — | A | A | A | — | — | A |
| | EC50 | A | — | — | — | A | A | A | — | A | A | A | — | — | A |
| 27 | MECP | A | — | — | — | A | A | A | — | A | A | A | — | — | A |
| | EC50 | A | — | — | — | A | A | A | — | A | A | A | — | — | A |
| 29 | MECP | A | B | A | A | B | B | B | B | B | A | A | B | A | A |
| | EC50 | A | D | A | A | D | B | B | B | A | A | A | A | A | A |
| 30 | MECP | A | — | — | — | A | A | A | — | A | A | A | — | — | A |
| | EC50 | A | — | — | — | A | A | A | — | A | A | A | — | — | A |
| 32 | MECP | A | — | — | — | A | A | A | — | B | A | B | — | — | A |
| | EC50 | A | — | — | — | B | B | B | — | A | A | B | — | — | B |
| 34 | MECP | A | B | A | A | B | A | A | B | A | A | A | A | A | A |
| | EC50 | A | D | A | A | D | A | B | D | A | A | A | A | A | A |
| 35 | MECP | A | — | — | — | A | A | A | — | A | A | A | — | — | A |
| | EC50 | A | — | — | — | A | A | A | — | A | A | A | — | — | A |
| 36 | MECP | A | B | A | A | B | A | — | A | A | A | A | A | A | A |
| | EC50 | A | D | A | A | D | A | — | A | A | A | A | A | A | A |
| 37 | MECP | A | B | A | A | B | A | A | B | A | A | A | A | A | A |
| | EC50 | A | D | A | A | D | A | A | D | A | A | A | A | A | A |
| 38 | MECP | A | — | — | — | A | A | A | — | A | A | A | — | — | A |
| | EC50 | A | — | — | — | A | A | A | — | A | A | A | — | — | A |
| 39 | MECP | A | B | A | A | B | A | — | A | A | A | A | A | A | A |
| | EC50 | A | D | D | B | D | A | — | A | A | A | A | A | A | A |
| 40 | MECP | A | — | — | — | A | A | A | — | A | A | A | — | — | A |
| | EC50 | A | — | — | — | A | A | A | — | A | A | A | — | — | A |
| 42 | MECP | A | — | — | — | A | A | A | — | A | A | A | — | — | A |
| | EC50 | A | — | — | — | A | A | A | — | A | A | A | — | — | A |
| 43 | MECP | A | — | — | — | A | A | A | — | A | A | A | — | — | A |
| | EC50 | A | — | — | — | A | A | A | — | A | A | A | — | — | A |

### Example 97. Xenograft Assays

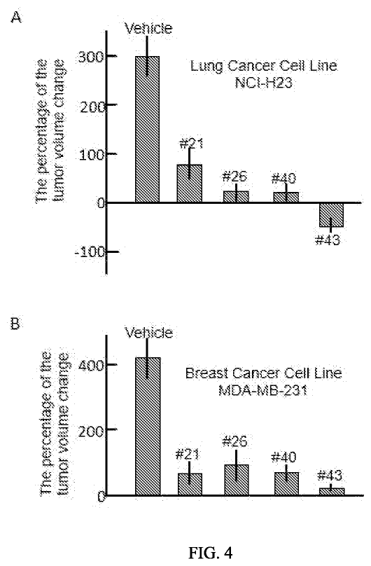

Xenografts were initiated in immunocompromised (Nu/Nu) mice with the human lung adenocarcinoma cell line NCI-H23 (FIG. 4A) and the human breast cancer cell line MDA-MB-231 (FIG. 4B). Seven million cells were injected subcutaneously into each mouse and treatment was initiated when the average tumor volumes reached 150 mm3 (n=5/group). Tumor-bearing mice were randomized into different groups to receive either vehicle or indicated compounds. The compounds were administered through oral gavage twice a day for 7 days. For these experiments, all compounds were first dissolved in DMSO and then diluted 1:10 into a mixture containing 50% PEG300 and 49% PBS, 1% Tween 80, pH2.2. 100 ul of drug solution was administered with each dose to each a dose of 25 mg/kg. Tumor volumes were determined from digital caliper raw data by using the formula: Volume (mm3)=(L×W²)/2. The value W (Width) is the smaller of two perpendicular tumor axes and the value L (Length) is the larger of two perpendicular axes. Mean tumor volumes were calculated for each treatment group at the start point (day 0) and the endpoint (day 7). The percentage change of tumor volumes, defined as (Tumor Volume^{day8}˜Tumor Volume^{day0})/Tumor Volume^{day0}×100% is presented. Compounds #21, #26, #40 and #43 demonstrated the therapeutic efficacy in both mouse tumor models, suppressing the tumor growth and even eliciting tumor regression by compound #43 (FIG. 4).

1. A compound of Formula (I) 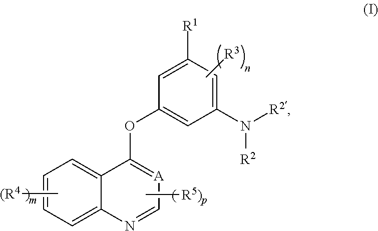
or a pharmaceutically acceptable salt or N-oxide thereof,
wherein
A is —C(H)═ or —N═
R¹ is selected from the group consisting of halogen, optionally substituted C₁-C₆ alkyl, optionally substituted C₁-C₆ alkoxy, optionally substituted C₁-C₆ haloalkoxy, —C(O)R^{a}, and —C(O)OR^{a};
R² is selected from the group consisting of hydrogen, optionally substituted C₁-C₆ aliphatic, optionally substituted C₁-C₆ haloaliphatic, —C(O)R^{a}, and —C(O)OR^{a};
R^{2′} is selected from the group consisting of optionally substituted C₁-C₆ aliphatic, optionally substituted C₁-C₆ haloaliphatic, —C(O)R^{a}, and —C(O)OR^{a};
optionally, R² and R^{2′} are taken together with the nitrogen on which they are attached to form optionally substituted 3-7-membered heterocyclyl or optionally substituted 5-9-membered heteroaryl;
each R³ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, and —C(O)N(R^{a})₂;
each R⁴ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, —C(O)N(R^{a})₂, optionally substituted phenyl, optionally substituted 3-7-membered heterocyclyl and optionally substituted 5-9-membered heteroaryl;
each R⁵ is independently selected from the group consisting of deuterium and halogen;
each R^{a} is independently is selected from the group consisting of hydrogen, optionally substituted C₁-C₆ aliphatic, optionally substituted C₁-C₆ haloaliphatic, optionally substituted 3-7-membered heterocyclyl, optionally substituted 5-9-membered heteroaryl, —C(O)R^{b}, and —C(O)OR^{b};
optionally, two instances of R^{a} are taken together with the nitrogen on which they are attached to form optionally substituted 3-7-membered heterocyclyl or optionally substituted 5-9-membered heteroaryl;
each R^{b} is independently optionally substituted C₁-C₆ aliphatic;
n is 0, 1, 2, or 3;
m is 0, 1, 2, 3, or 4; and
p is 0, 1, 2, or 3.

2. The compound of claim 1, wherein R² and R^{2′} are taken together with the nitrogen on which they are attached to form optionally substituted 3-7-membered heterocyclyl or optionally substituted 5-9-membered heteroaryl.

3. The compound of claim 2, wherein R² and R^{2′} are taken together with the nitrogen on which they are attached to form o optionally substituted 5-9-membered heteroaryl.

4. The compound of claim 1, wherein the compound is a compound of Formula (I-a) 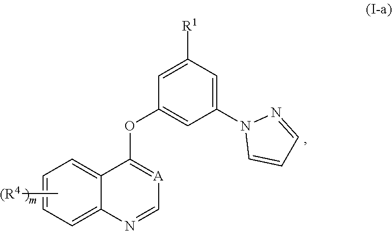
or a pharmaceutically acceptable salt or N-oxide thereof.

5. The compound of claim 1, wherein the compound is a compound of Formula (I-b): 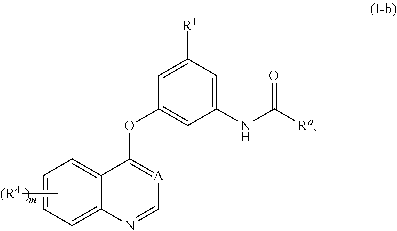
or a pharmaceutically acceptable salt or N-oxide thereof.

6. The compound of any of claim 1-5, wherein R¹ is optionally substituted C₁-C₆ alkoxy.

7. The compound of claim 6, wherein R¹ is —OMe.

8. A compound of Formula (II) 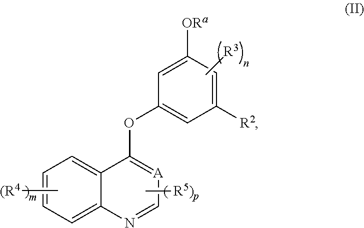
or a pharmaceutically acceptable salt or N-oxide thereof,
wherein
A is —C(H)═ or —N═
R² is selected from the group consisting of —NH₂, —NO₂ —OR^{a}, —O(CH₂)₁₋₃R^{a}, —C(O)OR^{a}, —C(O)N(R^{a})₂, optionally substituted C₁-C₆ aliphatic, and optionally substituted 5-9-membered heteroaryl;
each R³ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, and —C(O)N(R^{a})₂;
each R⁴ is independently selected from the group consisting of halogen, —CN, —OR^{a}, —N(R^{a})₂, —NO₂, optionally substituted C₁-C₆ aliphatic, optionally substituted C₁-C₆ haloaliphatic, —C(O)R^{a}, —C(O)OR^{a}, —C(O)N(R^{a})₂, optionally substituted phenyl, optionally substituted 3-7-membered heterocyclyl and optionally substituted 5-9-membered heteroaryl;
each R⁵ is independently selected from the group consisting of deuterium and halogen;
each R^{a} is independently is selected from the group consisting of hydrogen, optionally substituted C₁-C₆ aliphatic, optionally substituted C₁-C₆ haloaliphatic, optionally substituted 3-7-membered heterocyclyl, optionally substituted 5-9-membered heteroaryl, —C(O)R^{b}, and —C(O)OR^{b};
optionally, two instances of R^{a} are taken together with the nitrogen on which they are attached to form optionally substituted 3-7-membered heterocyclyl or optionally substituted 5-9-membered heteroaryl;
each R^{a} is independently is selected from the group consisting of optionally substituted C₁-C₆ aliphatic and optionally substituted C₁-C₆ haloaliphatic;
n is 0, 1, 2, or 3;
m is 0, 1, 2, 3, or 4; and
p is 0, 1, 2, or 3.

9. The compound of claim 8, wherein the compound is of Formula (II-a) 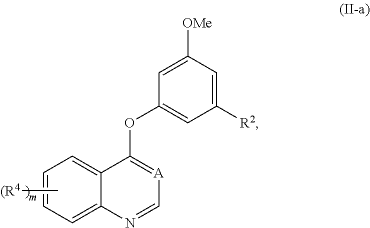
or a pharmaceutically acceptable salt or N-oxide thereof.

10. The compound of any of claims 8-9, wherein R² is —C(O)OR^{a}, and R^{a} of R² is C₁-C₆ aliphatic.

11. The compound of any of claims 8-9, wherein R² is —C(O)NHR^{a}, and R^{a} of R² is C₁-C₆ aliphatic.

12. The compound of any of claims 8-9, wherein R² is optionally substituted 5-membered heteroaryl.

13. The compound of any of claims 8-9, wherein R² is optionally substituted C₁-C₆ aliphatic.

14. The compound of claim 13, wherein R² is C₁-C₆ substituted with 1-7 instances of fluoro.

15. The compound of claim 14, wherein R² is —CF₃.

16. A compound of Formula (III) 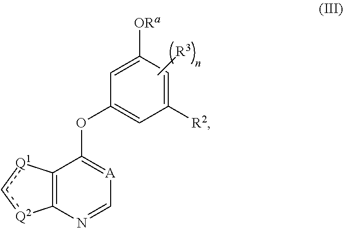
or a pharmaceutically acceptable salt or N-oxide thereof,
wherein
A is —C(H)═ or —N═;
one of Q¹ and Q² is —N(R^{a})— or —S— and the other is —C(H)═;
R² is selected from the group consisting of —C(O)OR^{a}, —C(O)N(R^{a})₂, optionally substituted C₁-C₆ haloaliphatic, and optionally substituted 5-9-membered heteroaryl;
each R³ is independently selected from the group consisting of halogen, —CN, —OR^{a}, —N(R^{a}), —NO₂, optionally substituted C₁-C₆ aliphatic, optionally substituted C₁-C₆ haloaliphatic, —C(O)R^{a}, —C(O)OR^{a}, and —C(O)N(R^{a})₂;
each R^{a} is independently is selected from the group consisting of hydrogen, optionally substituted C₁-C₆ aliphatic, optionally substituted C₁-C₆ haloaliphatic, —C(O)R^{b}, and —C(O)OR^{b};
optionally, two instances of R^{a} are taken together with the nitrogen on which they are attached to form optionally substituted 3-7-membered heterocyclyl or optionally substituted 5-9-membered heteroaryl;
each R^{a} is independently is selected from the group consisting of optionally substituted C₁-C₆ aliphatic and optionally substituted C₁-C₆ haloaliphatic;
n is 0, 1, 2, or 3;
m is 0, 1, 2, 3, or 4.

17. The compound of claim 16, wherein the compound is of Formula (III-a1) or (III-a2) 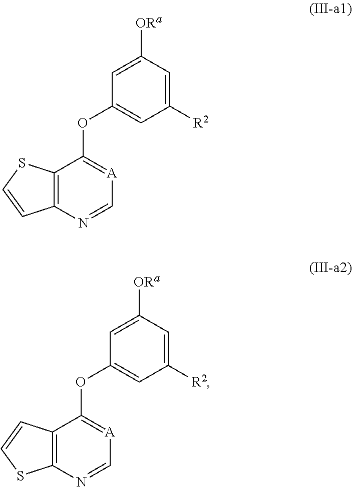
or a pharmaceutically acceptable salt or N-oxide thereof.

18. The compound of claim 16, wherein the compound is of Formula (III-b1) or (III-b2) 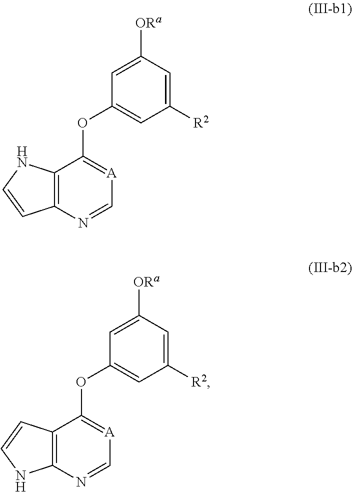
or a pharmaceutically acceptable salt or N-oxide thereof.

19. The compound of any of claims 16-18, wherein R² is —C(O)OR^{a} or optionally substituted 5-9-membered heteroaryl.

20. The compound of claim 19, wherein R² is —C(O)OMe or pyrazole.

21. A compound selected from the group consisting of:

| NO. | Structure |
|---|---|
| 1 | 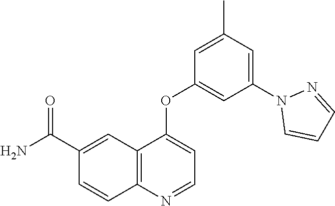 [SMILES: Cc1cc(Oc2ccnc3ccc(C(N)=O)cc23)cc(-n2cccn2)c1] |
| 2 | 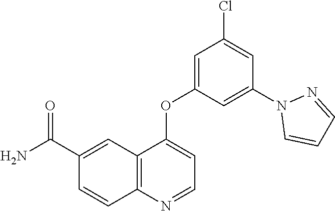 [SMILES: NC(=O)c1ccc2nccc(Oc3cc(Cl)cc(-n4cccn4)c3)c2c1] |
| 3 | 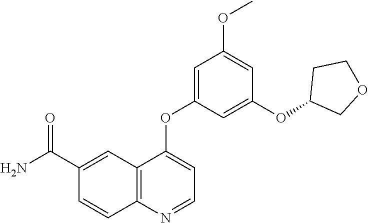 [SMILES: COc1cc(Oc2ccnc3ccc(C(N)=O)cc23)cc(O[C@@H]2CCOC2)c1] |
| 4 | 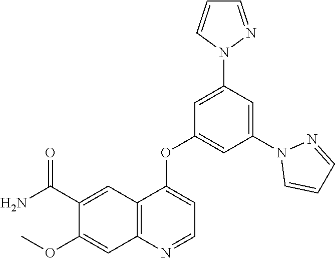 [SMILES: COc1cc2nccc(Oc3cc(-n4cccn4)cc(-n4cccn4)c3)c2cc1C(N)=O] |
| 5 | 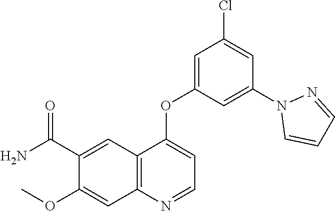 [SMILES: COc1cc2nccc(Oc3cc(Cl)cc(-n4cccn4)c3)c2cc1C(N)=O] |
| 6 | 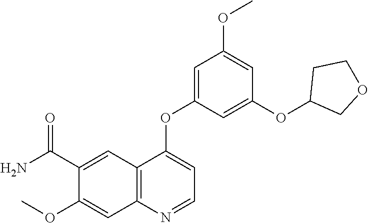 [SMILES: COc1cc(Oc2ccnc3cc(OC)c(C(N)=O)cc23)cc(OC2CCOC2)c1] |
| 7 | 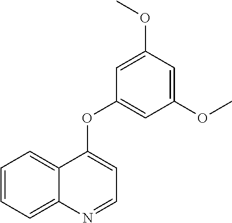 [SMILES: COc1cc(OC)cc(Oc2ccnc3ccccc23)c1] |
| 8 | 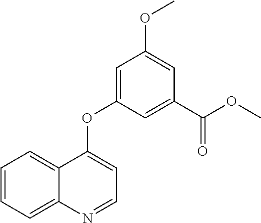 [SMILES: COC(=O)c1cc(OC)cc(Oc2ccnc3ccccc23)c1] |
| 9 | 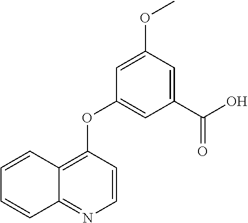 [SMILES: COc1cc(Oc2ccnc3ccccc23)cc(C(=O)O)c1] |
| 10 | 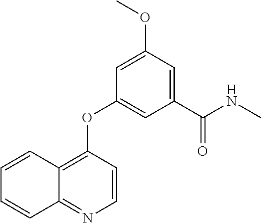 [SMILES: CNC(=O)c1cc(OC)cc(Oc2ccnc3ccccc23)c1] |
| 11 | 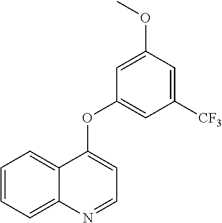 [SMILES: COc1cc(Oc2ccnc3ccccc23)cc(C(F)(F)F)c1] |
| 12 | 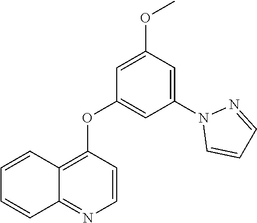 [SMILES: COc1cc(Oc2ccnc3ccccc23)cc(-n2cccn2)c1] |
| 13 | 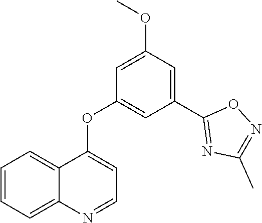 [SMILES: COc1cc(Oc2ccnc3ccccc23)cc(-c2nc(C)no2)c1] |
| 14 | 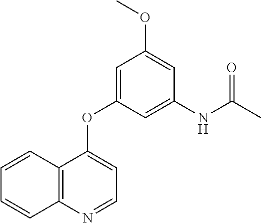 [SMILES: COc1cc(NC(C)=O)cc(Oc2ccnc3ccccc23)c1] |
| 15 | 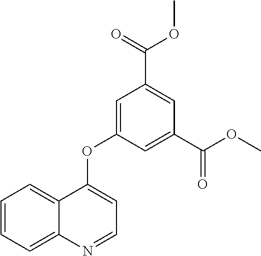 [SMILES: COC(=O)c1cc(Oc2ccnc3ccccc23)cc(C(=O)OC)c1] |
| 17 | 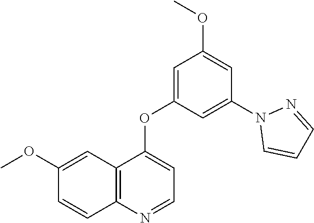 [SMILES: COc1cc(Oc2ccnc3ccc(OC)cc23)cc(-n2cccn2)c1] |
| 18 | 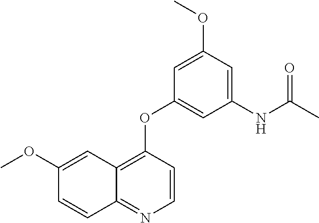 [SMILES: COc1cc(NC(C)=O)cc(Oc2ccnc3ccc(OC)cc23)c1] |
| 19 | 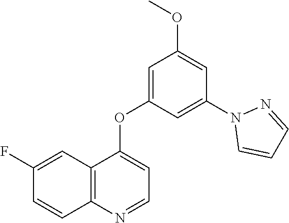 [SMILES: COc1cc(Oc2ccnc3ccc(F)cc23)cc(-n2cccn2)c1] |
| 20 | 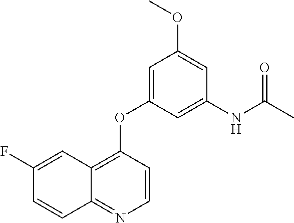 [SMILES: COc1cc(NC(C)=O)cc(Oc2ccnc3ccc(F)cc23)c1] |
| 21 | 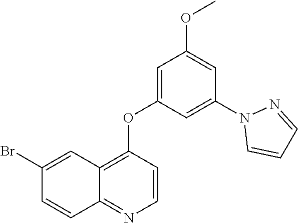 [SMILES: COc1cc(Oc2ccnc3ccc(Br)cc23)cc(-n2cccn2)c1] |
| 22 | 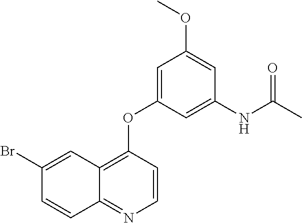 [SMILES: COc1cc(NC(C)=O)cc(Oc2ccnc3ccc(Br)cc23)c1] |
| 26 | 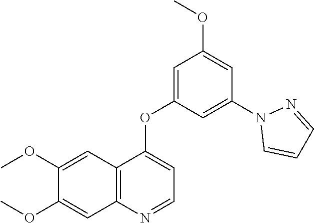 [SMILES: COc1cc(Oc2ccnc3cc(OC)c(OC)cc23)cc(-n2cccn2)c1] |
| 27 | 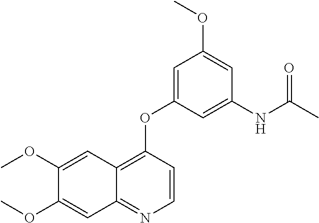 [SMILES: COc1cc(NC(C)=O)cc(Oc2ccnc3cc(OC)c(OC)cc23)c1] |
| 29 | 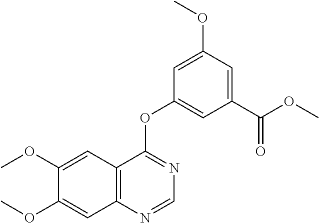 |
| 30 | 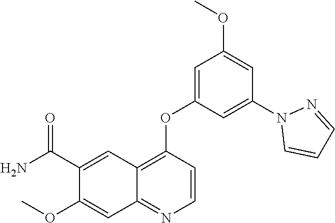 [SMILES: COc1cc(Oc2ccnc3cc(OC)c(C(N)=O)cc23)cc(-n2cccn2)c1] |
| 31 | 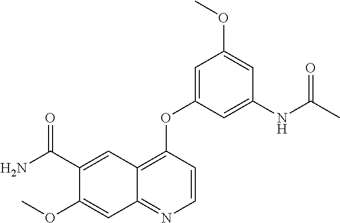 [SMILES: COc1cc(NC(C)=O)cc(Oc2ccnc3cc(OC)c(C(N)=O)cc23)c1] |
| 32 | 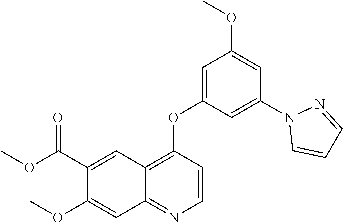 [SMILES: COC(=O)c1cc2c(Oc3cc(OC)cc(-n4cccn4)c3)ccnc2cc1OC] |
| 34 | 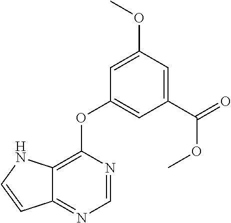 [SMILES: COC(=O)c1cc(OC)cc(Oc2ncnc3cc[nH]c23)c1] |
| 35 | 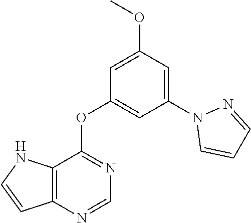 [SMILES: COc1cc(Oc2ncnc3cc[nH]c23)cc(-n2cccn2)c1] |
| 36 | 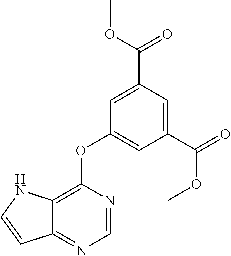 [SMILES: COC(=O)c1cc(Oc2ncnc3cc[nH]c23)cc(C(=O)OC)c1] |
| 37 | 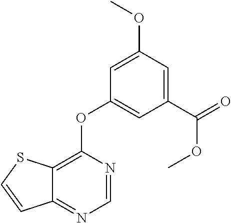 [SMILES: COC(=O)c1cc(OC)cc(Oc2ncnc3ccsc23)c1] |
| 38 | 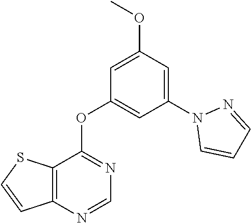 [SMILES: COc1cc(Oc2ncnc3ccsc23)cc(-n2cccn2)c1] |
| 39 | 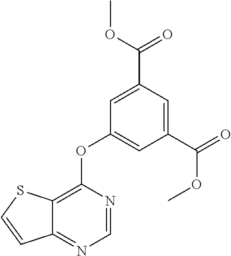 [SMILES: COC(=O)c1cc(Oc2ncnc3ccsc23)cc(C(=O)OC)c1] |
| 40 | 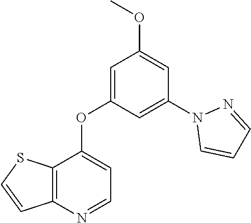 [SMILES: COc1cc(Oc2ccnc3ccsc23)cc(-n2cccn2)c1] |
| 41 | 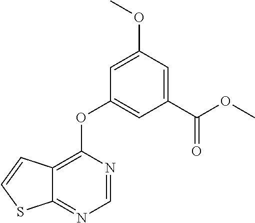 [SMILES: COC(=O)c1cc(OC)cc(Oc2ncnc3sccc23)c1] |
| 42 | 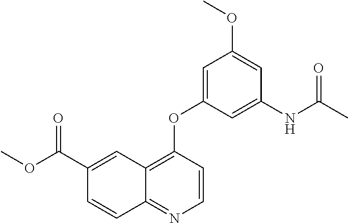 [SMILES: COC(=O)c1ccc2nccc(Oc3cc(NC(C)=O)cc(OC)c3)c2c1] |
| 43 | 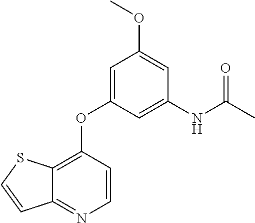 [SMILES: COc1cc(NC(C)=O)cc(Oc2ccnc3ccsc23)c1] |
| 44 | 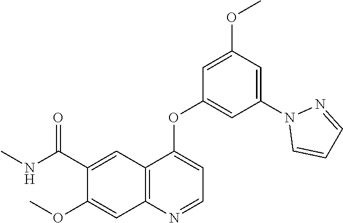 [SMILES: CNC(=O)c1cc2c(Oc3cc(OC)cc(-n4cccn4)c3)ccnc2cc1OC] |
| 45 | 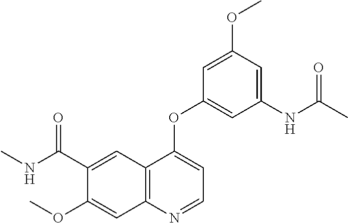 [SMILES: CNC(=O)c1cc2c(Oc3cc(NC(C)=O)cc(OC)c3)ccnc2cc1OC] |
| 46 | 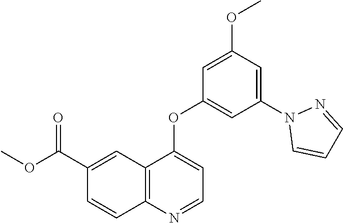 [SMILES: COC(=O)c1ccc2nccc(Oc3cc(OC)cc(-n4cccn4)c3)c2c1] |
| 47 | 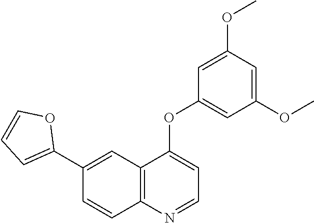 [SMILES: COc1cc(OC)cc(Oc2ccnc3ccc(-c4ccco4)cc23)c1] |
| 48 | 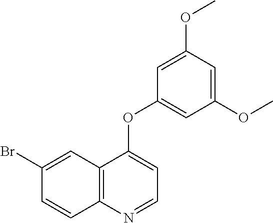 [SMILES: COc1cc(OC)cc(Oc2ccnc3ccc(Br)cc23)c1] |
| 49 | 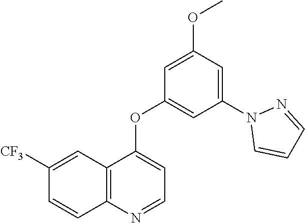 [SMILES: COc1cc(Oc2ccnc3ccc(C(F)(F)F)cc23)cc(-n2cccn2)c1] |
| 50 | 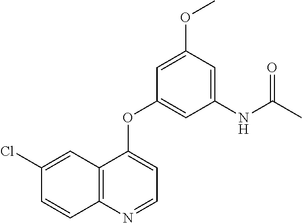 [SMILES: COc1cc(NC(C)=O)cc(Oc2ccnc3ccc(Cl)cc23)c1] |
| 51 | 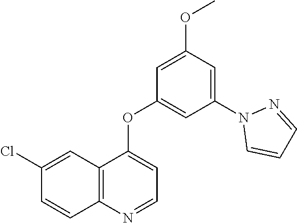 [SMILES: COc1cc(Oc2ccnc3ccc(Cl)cc23)cc(-n2cccn2)c1] |
| 52 | 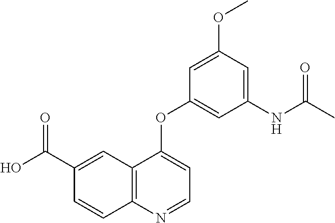 [SMILES: COc1cc(NC(C)=O)cc(Oc2ccnc3ccc(C(=O)O)cc23)c1] |
| 53 | 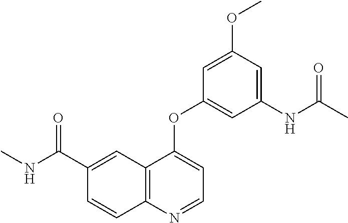 [SMILES: CNC(=O)c1ccc2nccc(Oc3cc(NC(C)=O)cc(OC)c3)c2c1] |
| 54 | 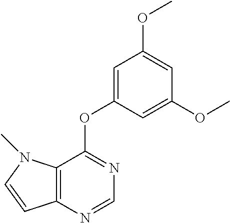 |
| 55 | 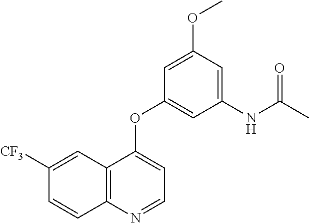 [SMILES: COc1cc(NC(C)=O)cc(Oc2ccnc3ccc(C(F)(F)F)cc23)c1] |
| 56 | 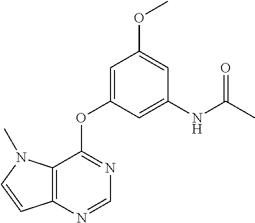 [SMILES: COc1cc(NC(C)=O)cc(Oc2ncnc3ccn(C)c23)c1] |
| 57 | 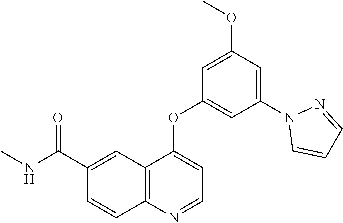 [SMILES: CNC(=O)c1ccc2nccc(Oc3cc(OC)cc(-n4cccn4)c3)c2c1] |
| 58 | 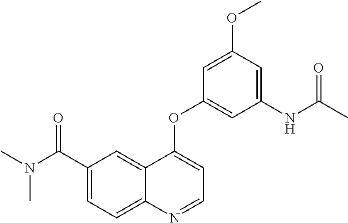 [SMILES: COc1cc(NC(C)=O)cc(Oc2ccnc3ccc(C(=O)N(C)C)cc23)c1] |
| 59 | 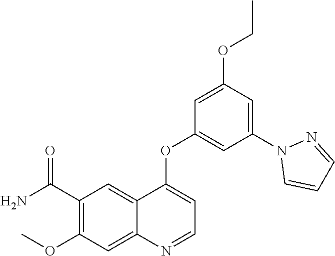 [SMILES: CCOc1cc(Oc2ccnc3cc(OC)c(C(N)=O)cc23)cc(-n2cccn2)c1] |
| 60 | 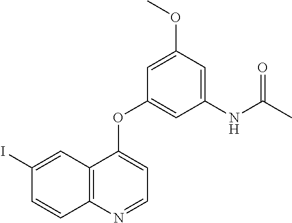 [SMILES: COc1cc(NC(C)=O)cc(Oc2ccnc3ccc(I)cc23)c1] |
| 61 | 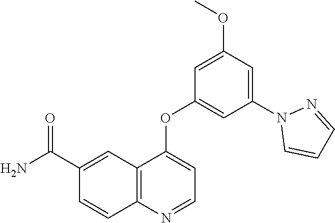 [SMILES: COc1cc(Oc2ccnc3ccc(C(N)=O)cc23)cc(-n2cccn2)c1] |
| 62 | 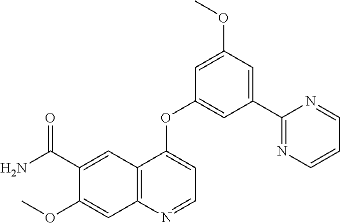 [SMILES: COc1cc(Oc2ccnc3cc(OC)c(C(N)=O)cc23)cc(-c2ncccn2)c1] |
| 63 | 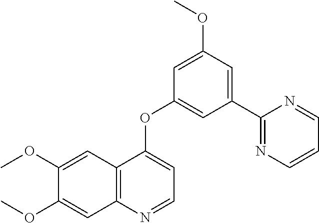 [SMILES: COc1cc(Oc2ccnc3cc(OC)c(OC)cc23)cc(-c2ncccn2)c1] |
| 64 | 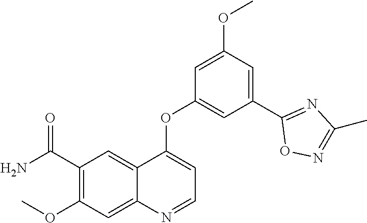 [SMILES: COc1cc(Oc2ccnc3cc(OC)c(C(N)=O)cc23)cc(-c2nc(C)no2)c1] |
| 65 | 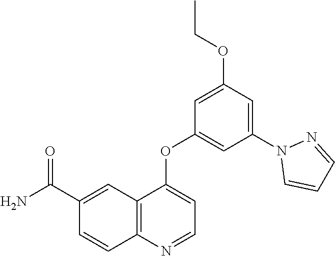 [SMILES: CCOc1cc(Oc2ccnc3ccc(C(N)=O)cc23)cc(-n2cccn2)c1] |
| 66 | 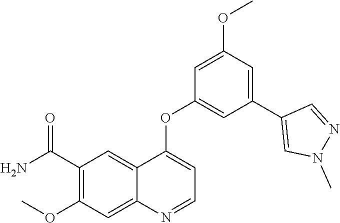 [SMILES: COc1cc(Oc2ccnc3cc(OC)c(C(N)=O)cc23)cc(-c2cnn(C)c2)c1] |
| 67 | 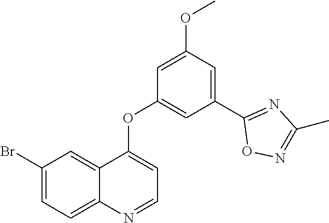 [SMILES: COc1cc(Oc2ccnc3ccc(Br)cc23)cc(-c2nc(C)no2)c1] |
| 68 | 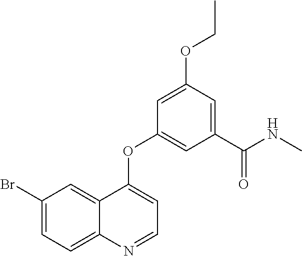 [SMILES: CCOc1cc(Oc2ccnc3ccc(Br)cc23)cc(C(=O)NC)c1] |
| 69 | 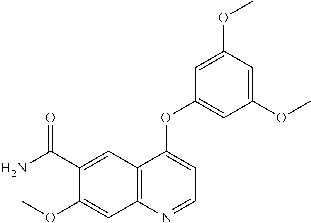 [SMILES: COc1cc(OC)cc(Oc2ccnc3cc(OC)c(C(N)=O)cc23)c1] |
| 70 | 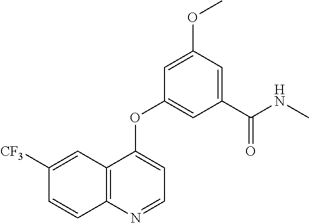 [SMILES: CNC(=O)c1cc(OC)cc(Oc2ccnc3ccc(C(F)(F)F)cc23)c1] |
| 71 | 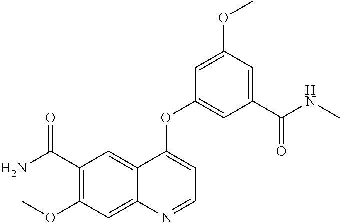 [SMILES: CNC(=O)c1cc(OC)cc(Oc2ccnc3cc(OC)c(C(N)=O)cc23)c1] |
| 72 | 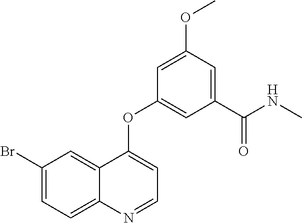 [SMILES: CNC(=O)c1cc(OC)cc(Oc2ccnc3ccc(Br)cc23)c1] |
| 73 | 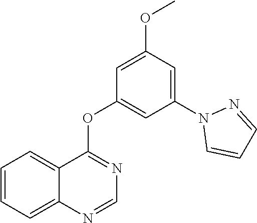 [SMILES: COc1cc(Oc2ncnc3ccccc23)cc(-n2cccn2)c1] |
| 74 | 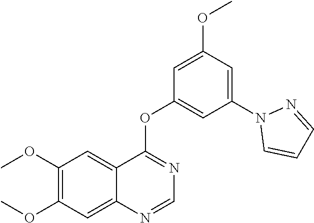 [SMILES: COc1cc(Oc2ncnc3cc(OC)c(OC)cc23)cc(-n2cccn2)c1] |
| 75 | 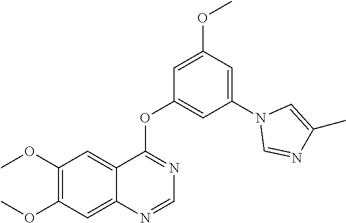 |
| 76 | 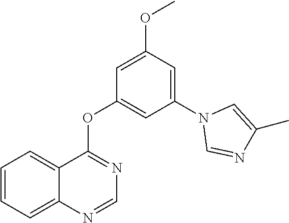 [SMILES: COc1cc(Oc2ncnc3ccccc23)cc(-n2cnc(C)c2)c1] |
| 77 | 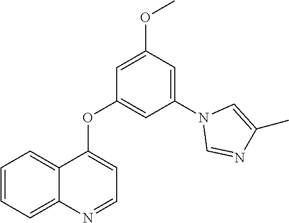 [SMILES: COc1cc(Oc2ccnc3ccccc23)cc(-n2cnc(C)c2)c1] |
| 78 | 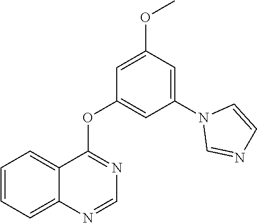 [SMILES: COc1cc(Oc2ncnc3ccccc23)cc(-n2ccnc2)c1] |
| 79 | 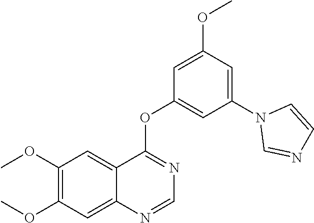 [SMILES: COc1cc(Oc2ncnc3cc(OC)c(OC)cc23)cc(-n2ccnc2)c1] |
| 80 | 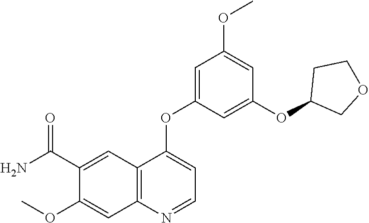 [SMILES: COc1cc(Oc2ccnc3cc(OC)c(C(N)=O)cc23)cc(O[C@H]2CCOC2)c1] |
| 81 | 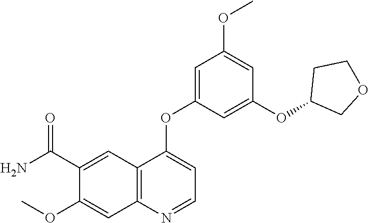 [SMILES: COc1cc(Oc2ccnc3cc(OC)c(C(N)=O)cc23)cc(O[C@@H]2CCOC2)c1] |
| 82 | 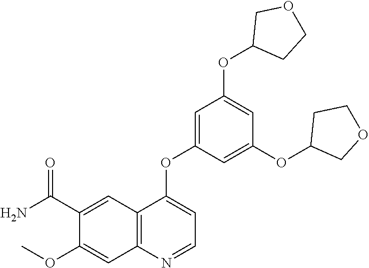 [SMILES: COc1cc2nccc(Oc3cc(OC4CCOC4)cc(OC4CCOC4)c3)c2cc1C(N)=O] |
| 83 | 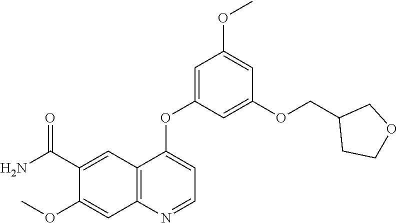 [SMILES: COc1cc(OCC2CCOC2)cc(Oc2ccnc3cc(OC)c(C(N)=O)cc23)c1] |
| 84 | 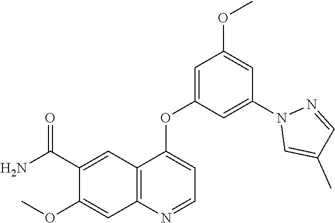 [SMILES: COc1cc(Oc2ccnc3cc(OC)c(C(N)=O)cc23)cc(-n2cc(C)cn2)c1] |
| 85 | 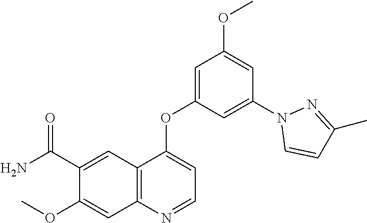 [SMILES: COc1cc(Oc2ccnc3cc(OC)c(C(N)=O)cc23)cc(-n2ccc(C)n2)c1] |
| 86 | 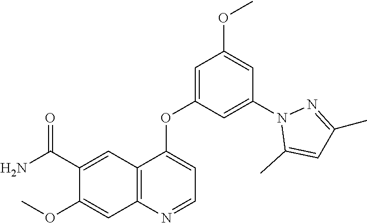 [SMILES: COc1cc(Oc2ccnc3cc(OC)c(C(N)=O)cc23)cc(-n2nc(C)cc2C)c1] |
| 87 | 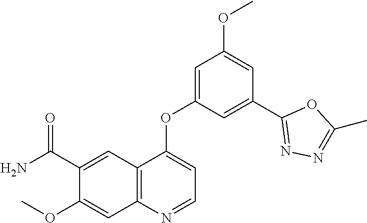 [SMILES: COc1cc(Oc2ccnc3cc(OC)c(C(N)=O)cc23)cc(-c2nnc(C)o2)c1] |
| 88 | 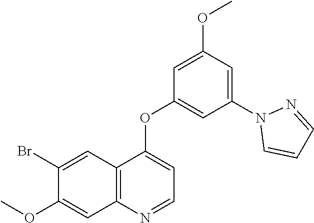 [SMILES: COc1cc(Oc2ccnc3cc(OC)c(Br)cc23)cc(-n2cccn2)c1] |
| 89 | 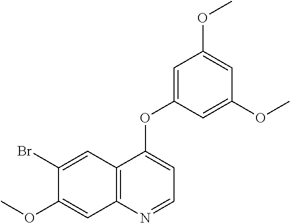 |
| 90 | 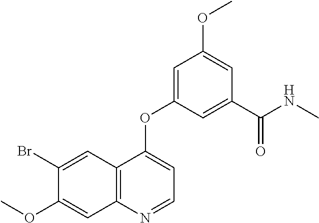 |
| 91 | 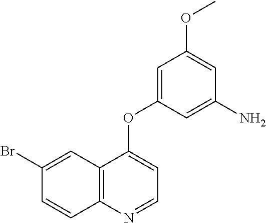 [SMILES: COc1cc(N)cc(Oc2ccnc3ccc(Br)cc23)c1] |
| 92 | 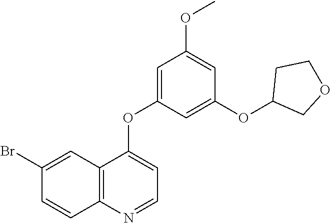 [SMILES: COc1cc(Oc2ccnc3ccc(Br)cc23)cc(OC2CCOC2)c1] |
| 93 | 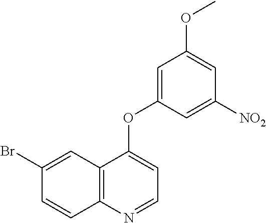 [SMILES: COc1cc(Oc2ccnc3ccc(Br)cc23)cc([N+](=O)[O-])c1] |
| 94 | 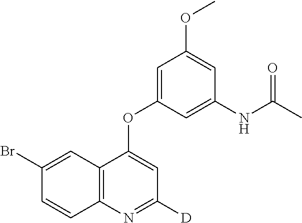 [SMILES: [2H]c1cc(Oc2cc(NC(C)=O)cc(OC)c2)c2cc(Br)ccc2n1] |
| 95 | 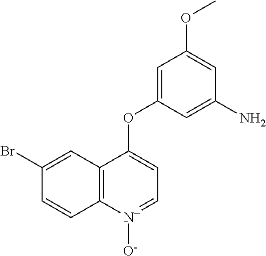 [SMILES: COc1cc(N)cc(Oc2cc[n+]([O-])c3ccc(Br)cc23)c1] |
| 96 | 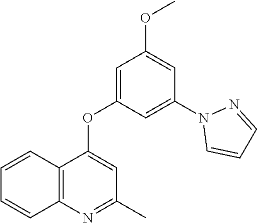 [SMILES: COc1cc(Oc2cc(C)nc3ccccc23)cc(-n2cccn2)c1] |
| 97 | 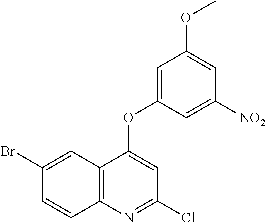 [SMILES: COc1cc(Oc2cc(Cl)nc3ccc(Br)cc23)cc([N+](=O)[O-])c1] |
| 98 | 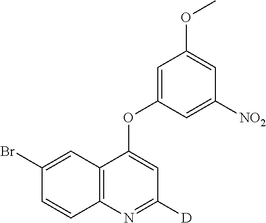 [SMILES: [2H]c1cc(Oc2cc(OC)cc([N+](=O)[O-])c2)c2cc(Br)ccc2n1] |

or a pharmaceutically acceptable salt thereof.

22. A pharmaceutical composition comprising a compound of any of the previous claims and a pharmaceutically acceptable excipient.

23. A method of treat cancer comprising administering to a patient in need thereof the compound of any of claims 1-21 or the pharmaceutical composition of claim 22.
